SEQUENCE LISTING (27 sequences)

SEQ ID NO: 1 - DNA, 3348 bp, Mus musculus
gggcacgtgggatcggctgcagcactgcgccagttcagcccgcctagcagcgagcggccgcggcctctggagagaggttcctccccctctgctctgcaccagtccgagggaatctgtggtcagtgacgcgcatccttcagcgagccacctgcagcccggggcgcgccgctgggaccgcatcacaggctgggccggcggcctggctaccgcgcgctccggaggccatgcgggcgttggcgcagcgcagcgaccggcggctgctgctgcttgttgttttgtcagtaatgattcttgagaccgttacaaaccaagacctgcctgtgatcaagtgtgttttaatcagtcatgagaacaatggctcatcagcgggaaagccatcatcgtaccgaatgaggaatcgtttccatggccatcttgaacgtgacagagacccaggcaggagaatacctactccatattcagagcgaagccgccaactacacagtactgttcacagtgaatgtaagagatacacagctgtacgtgctaagaagaccttactttaggaagatggaaaaccaggacgcactgctctgcatctccgagggtgttccagagcccactgtggagtgggtgctctgcagctcccacagggaaagctgtaaagaagaaggccctgctgttgtcagaaaggaggaaaaggtacttcatgagttgttcggaacagacatcagatgctgtgctagaaatgcactgggccgcgaatgcaccaagctgttcaccatagatctaaaccaggctcctcagagcacactgccccagttattcctgaaagtgggggaacccttgtggatcaggtgtaaggccatccatgtgaaccatggattcgggctcacctgggagctggaagacaaagccctggaggagggcagctactttgagatgagtacctactccacaaacaggaccatgattcggattctcttggcctttgtgtcttccgtgggaaggaacgacaccggatattacacctgctcttcctcaaagcaccccagccagtcagcgttggtgaccatcctagaaaaagggtttataaacgctaccagctcgcaagaagagtatgaaattgacccgtacgaaaagttctgcttctcagtcaggtttaaagcgtacccacgaatccgatgcacgtggatcttctctcaagcctcatttccttgtgaacagagaggcctggaggatgggtacagcatatctaaattttgcgatcataagaacaagccaggagagtacatattctatgcagaaaatgatgacgcccagttcaccaaaatgttcacgctgaatataagaaagaaacctcaagtgctagcaaatgcctcagccagccaggcgtcctgttcctctgatggctacccgctaccctcttggacctggaagaagtgttcggacaaatctcccaattgcacggaggaaatcccagaaggagtttggaataaaaaggctaacagaaaagtgtttggccagtgggtgtcgagcagtactctaaatatgagtgaggccgggaaagggcttctggtcaaatgctgtgcgtacaattctatgggcacgtcttgcgaaaccatctttttaaactcaccaggccccttccctttcatccaagacaacatctccttctatgcgaccattgggctctgtctccccttcattgttgttctcattgtgttgatctgccacaaatacaaaaagcaatttaggtacgagagtcagctgcagatgatccaggtgactggccccctggataacgagtacttctacgttgacttcagggactatgaatatgaccttaagtgggagttcccgagagagaacttagagtttgggaaggtcctggggtctggcgctttcgggagggtgatgaacgccacggcctatggcattagtaaaacgggagtctcaattcaggtggcggtgaagatgctaaaagagaaagctgacagctgtgaaaaagaagctctcatgtcggagctcaaaatgatgacccacctgggacaccatgacaacatcgtgaatctgctgggggcatgcacactgtcagggccagtgtacttgatttttgaatattgttgctatggtgacctcctcaactacctaagaagtaaaagagagaagtttcacaggacatggacagagatttttaaggaacataatttcagtttttaccctactttccaggcacattcaaattccagcttcagaatgaattaaattcccattgaaccctgagagctgatccaagggcgggtgtaactgaacttctcgtgaaccaggcatgatgagattgaatatgaaaaccagaagaggctggcagaagaagaggaggaagatttgaacgtgctgacgtttgaagacctcctttgctttgcgtaccaagtggccaaaggcatggaattcctggagttcaagtcgtgtgtccacagagacctggcagccaggaatgtgttggtcacccacgggaaggtggtgaagatctgtgactttggactggcccgagacatcctgagcgactccagctacgtcgtcaggggcaacgcacggctgccggtgaagtggatggcacctgagagcttatttgaagggatctacacaatcaagagtgacgtctggtcctacggcatccttctctgggagatattttcactgggtgtgaacccttaccctggcattcctgtcgacgctaacttctataaactgattcagagtggatttaaaatggagcagccattctatgccacagaagggatatgtatcagaacatgggtggcaacgtcccagaacatccatccatctaccaaaacaggcggcccctcagcagagaggcaggctcagagccgccatcgccacaggcccaggtgaagattcacggagaaagaagttagcgaggaggccttggaccccgccaccctagcaggctgtagaccacagagccaagattagcctcgcctctgaggaagcgccctacaggccgttgcttcgctggacttttctctagatgctgtctgccattactccaaagtgacttctataaaatcaaacctctcctcgcacaggtgggagagccaataatgagacttgttggtgagcccgcctaccctggggggcctttccaggccccccaggcttgaggggaaagccatgtatctgaaatatagtatattcttgtaaatacgtgaaacaaaccaaacccgttttttgctaagggaaagctaaatatgatttttaaaaatctatgttttaaaatactatgtaactttttcatctatttagtgatatattttatggatggaaataaactttctactgtagaaa

SEQ ID NO: 2 - DNA, 6770 bp, Mus musculus
tcatggatgtatgctcccgacttaaaaagcaccttttttaaaaaactaaaaacagaaatctgaatgttgtagtaagtgtaacaatcttaagtttattcagtaatttaaaaaaattgttaagcggagaaaagaaactctgtactaacagaggcctgagaaagcacacggcagggaataggggaaatggcttccttcattgctggacacagactgagctccaggctgtttcagctgcctttttaaggctcaagggcactaaaagtaaaaccatcctgcttcctctccccattttcattttcacctaaaatcccctagtccctttgtgaagaccagggcttcacacggtgaaagaatggtggactcacttctttcaataggctgacctagtatgtacactaagtccacccatgttttaactttcttcctagtttattccccttctgatttcttcacaagaatcaaccggcttttcattttaatctactctaatcgcctgtgtgtttacactgggttacattctttagagtgtacttatattctccttttgcattctcaatataaattaatctgctagatataaagctgttctctttattttagtgtaatttttttcttcacattgaattctaggagaaactatgctagtgatatataattcttgaactattaaacatgggagcataagaaaacaagaatcttaaggcaatctgcagagtgaagaagctgattgtgatcctgagagtgtgttttgtaaatggttttggattttatgtacagagcctactttcagcctggaatcattctgaatgctagctagatatctggagacaggtggacagaaaaccaggaactagtctgaaaaagaaactaaccaaagggaagaagtctgtttaagtttgacccagcctagaagacttgagcattggaggggttattcagagtgagacgtaccaccttcagattcaaatcctgtcatccagtagaagggagcttcaaacacaagctagctaagatacaatgaggtccttcttcgatatctttatcttccatataccatgaatcaaagaaacttcaacaacatgaggactgcaacagaccttcaagcctccttgcatgacctggaaatgttttggggtgtcctggcagcagtgggatcagcactaacagataagggcaactctcacagagactaaaggtcttaactaagaagatagccaagagaccactggggagaatgcagagaataggcttggacttggaagccaagattgcttgacaacagacagaagatatttctgtacttcacccactttacccacctggcaactcctggaaacaactgcacaaaatttggaggtgaacaaaccattagaaacaactggtcctgacaagacacaggaaaaacaagcaatatgcaacattactgtctgttgtccaggttgggtgctgggggtgggagagggagtgtgtgtctttgtatgatctgaaaaaactcaggtcagaacatctgtagatccttacagacatacaaaagaatcctagcctcttattcacgtctgtcatgcgcgcgtgcctgcgtttaaataacatcagctttagcttctctttctccttataaaacattgtgaatttcagttttctttcccatcaagacatgctcaagtgctgagtcacttttaaaggaagagtgctcatgcttcttagggctagcctcaaggatgacttaagcacactttccccttcctagttgtgattctttcgatgctaaacgacgtcacattgtgcaatcttaataaggtttccaatcagccccacccactctggccccacccccaccctccaacaaagatttttatcaaatgtgggattttcccatgagtctcaaaattagagagttgactcctaataaatatgagactggggatgtctgtagctcattctgctctggagcccaccaagaacgatagtcaattccagaaaccgctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggagttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggtaggattcctcaaagccattccagctaagattcatacctcagagcccaccaaagtggcaaatcataaataggttaaagcatctccccactttcaatgcaaggtattttggtcctgttggcttgaattatattctcctaattattgtcaaaattgctgactggaatttgcttgccaggatgccaatgagttgtagcttcatttttcttagagactttcctggctgtggttgaacaatgaaaaggccctctagtggtgtttgttttagggacacttaggtgataacaattctggtattctttcccagacatgtaacaagagtaacatgtgtgaaagcagcaaagaggcactggcagaaaacaacctgaaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagctcattctccacagtgagataacctgcactgtcttctgattattttataaaaggaggttccagcccagcattaacaagggcaagagtgcaggaagaacatcaagggggacaatcagagaaggatccccattgccacattctagcatctgttgggctttggataaaactaattacatggggcctctgattgtccagttatttaaaatggtgctgtccaatgtcccaaaacatgctgcctaagaggtacttgaagttctctagaggagcagagggaaaagatgtcgaactgtggcaattttaacttttcaaattgattctatctcctggcgataaccaattttcccaccatctttcctcttaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagcaagtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgcttgatagtggtgcttccctcaggatgcttgtggtctaatgggagacagaacagcaaagggatgattagaagttggttgctgtgagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttccttcaggcaaagaatctagatgcaataaccacccctgacccaaccacaaatgccagcctgctgacgaagctgcaggcacagaaccagtggctgcaggacatgacaactcatctcattctgcgcagctttaaggagttctgcagtccagcctgagggctcttcggcaaatgtagtgcgttatgcctaagcatatcagtttgtggacattcctcactgtggtcagaaaatatatcctgttgtcaggtatctgacttatgttgttctctacgaagaactgacaatatgaatgttgggacactattttaattatttttaatttattgataatttaaataagtaaactttaagttaatttatgattgatatttattatttttatgaagtgtcacttgaaatgttatatgttatagttttgaaatgataacctaaaaatctatttgatataaatattctgttacctagccagatggtttcttggaatgtataagtttacctcaatgaattgctaatttaaatatgtttttaaagaaatctttgtgatgtatttttataatgtttagactgtcttcaaacaaataaattatattatatttaaaaaccagtgactgaaagacgcatctcagctggtaaagttcttacccaacatgagcaaggtcctaagttacatccaaacatcctcccccaaatcaataattaagcactttttatgacatgtaaagttaaataagaagtgaaagctgcagatggtgagtgagagatgccatgagaaagcattgcatataccacattagttaatttcaggtcttgtacattcttttctggacatgagagagtaagggatctaactaagccaccttttggaaacataaaacataatctctgatttgaattcaagtctacctccctctaggtccatttttaacttttagttgtaatttgaagacagatatagaaaaatctcaaaacattttaatatgaattatacacttagagttgatgtcacagattctgagaccatgggactacttagataagatatagctccaaaagataaaagcgccaaaataatatccagaagttctgcctccctcgtctggagtctccatgcactgcatacctcctattagtgtctgccattatatatcataccttaaaactgaaggagctttctatccaactagcatatgggtccctcaagaaagcagactctagtgttttaaccttttcgtgctatatataggtaaggagcctgaacaaaggagacccctataagtatttgctgaatgaaaagagaatagttaatcacagtataacaaaagtcagttcttggtaaatacagagcatttgggtgacattacagtgatgtgttattgtcttttaaaaaaagtagaaaagaatggaaatgaaacattttaaggatttctaaataaggggcagatacaagagtattttgggttttagcccagactatactgtagggggaaagcctgtctcaactttatcccaatttcatatatgctataacttaatgtggttcttcctatttctgtacaaaactgagaatttggtgccaattttatta

SEQ ID NO: 3 - DNA, 12632 bp, Mus musculus
tttctagaaggagaaagaggagggagaagtaaacaaagcacaaagaatgagaactatcattaatataagaaataaaaattaagaaagcaagtgaatgtttttctagtgaaagtgggaaaaaggatggttacagcatgggtcatcttctggtctccctgggtaagaaaattaccaaactccctgagtagtcacacagctccaatgacatcacttctatttcctaccaaagagaaggtgtcccagtcttaatccaacctaggatttcccaaactgcacatgtagatactgttcattccttcagcattaagtatttggattaagataaaaccaggaagctcttcagcccacaggaatttccaaaaatataccttggcccagtggttgctccaggtaagcctaagtagattccaagaaggtggcagcagtgagctaccaaaagaaatctccgtagcaagcttgtttcagtgggagacatccctgccgtggctttccggatatcagtagatctgaggaaactcagtttccccttctcgtgtgaataagctgcagaccttgctgtcgtctgcactgcctttcagtggtttgaaacctgaattactccgttgtctcagttgtctttttccccagttctaataatgatttctctatgtcctccctgtacctgctcacacttccttgtcccttgattccgttcttatcttcagtaggttttgtttgcctgattgcttccttgttttgtattttttttgggggggggcacttcgacgttattatattcacataaatgcttcagagcagagttaatgattactggacaaatcagttattacagaacatgccgggggggggggattgaagaggggggggggggagagaaggagggatggatggagagagagagagagagagagagagagaaatatgatgtaattaaacatcattaatgaaaaccccactgtacaaataggacgagcactgcgcaactcaaatcaacacctaagaaagtgagagtgtggaaggagtcaaaggaaaatatgaatagctacacaggctgatcccttgagggtatgtgacatctctcctgcagttccccaaccctggaatatgcatgacactccactgcagctctcttagagactctcccttctcctcccttcacatttagaatcccaccctggatttagtgtaaccagtgacttaagaaggtaccgcatatgggagacaaagatacaaaaatcctgaaagggttctggattattgggctcaggactcaattcatccgtgttatcacaattaaaagtagtctttccttaaaaaaagccttggtttctgcatctctgtgatcaaaatcccataacaaggtttggaagaggcaagtttgggaaaatttcagagtgtattaacttagaatactgttggagggaagcctgggtaaataaaggagataaggttagaaagaagacttgaagtcaacatgggagtgatgagtgagaatcttaaagtaactgagtctaccaaaagtcaatataattgaaatgacttaagatgtcacatcattaccagtaaggtagctggatgctatggtgtaggtgatgtgcttagcaaagagatgccttctaaaaatccctgaagggggccccatgcctgcctcagatttacctacacatacataaactatagacacactttaaaggagaaaccaaaaatggcaggtaggctgggtgcaccccaatgggtgccaagccaaaacttatgggggtcaggggacaggttgtctgttgctgtctgacattcttgcccccatcagcaattattcctgggcactgcaacacatgaatctacccaaaagattcgggcggagaggcaatatacatgaagtgactttaaagaccacgtgtttaccaataaagaagtgggttccctacaggggaaggcaagtgaatgaagatggcaaaatcagctgccatttcctttcttttgtctcttggaactatcccaattcagtgaccacatctggatctctacattgcttctgcctatgcaatatctagctgctgatcagaatcatatctgatgtcacgccagatgaatcaggctttggcatcttcccttatcactgtaagaagtagagatgggaagacgccatgatccagacatggtatcataacctaaatttaaatcttgcaggactccagaaaagtccgtctctaaagtcatcagcaaagcagaaactttctgagcctcctgccaccgctacaatcttttattcctcatcctaatgccagagaactgggtccagctgtgctgctccagctgttgaaggccttctgggaaaacttcacctctgactccagtctgtgctttcccccgaatagaatcatttaccaatccctgtgctcgctccttccctggctcagcgtggtctgtgacattttcagggactcacgtggagcacccaacatcatcgttctgagcagtgactcctaggaacttcccgaagacgagactgatgcaggctctgacacgcaaaagtggggagagtgaactgggtctcaggagggcctggggcagctggctgagctccaggagagtaggggttgggttcgtgtcaacagctgggcctttctttcctgctcccagtactgtactggcgctgctccaatcagaaggctgcgagacatcctctcaggctatccctgactcacttggctacttttatcttgtacttcctttcaaaccccaaccaggggagcgcaaatcttaacccaacccaccatccagcttctttctccatccctgacaatcgtgctgctgggacgcatgcctggggccatccaacgatttactggctgagagtctgagctgacacagctcaacaggtcagaagctgttcctcccctaggaggagagcatggtggacaggtctctctctagtggcttagacctgcaacagcaccatagcaccatacaccttaggagcccccactactcctggtaaggcatctttactccactgagacctaaataatgagtttcgagggcggctggatgcttgacttcatcattttaaaaatcttagtcactctgtagaccaggctggccttgaactcagaaatccatctgcctctgagtcccaagagctgggattaaaggcgtgcgccaccaccgcccagctaccagttttctttaatcaagcttaggcactcaccctgattctgagtttttgaagatgagactaactggtccttttctcatatatttcaatttctcattgttcctgtttccagtattctgacaacaactgcccggttccagtgaaatgccttcaacaaaagttacgttatcccaaggctgcattcattctccaaaatctgtcatacaggaacactgcgtttctcggtagccacgaagaggaacactgccagttcaaactggacaaaggagatagatggtcagggtgtgcatggtggaacagcatcagtagcaaacccctaaagtgactgcgggtgttagaaggtgtttttccaagcagaaaaaaaatcagtcatagaaactgcccagtaggaaaaagatgtcaaaatgatgacatggtatcatctctaaaagcatatcgaagcatgtagcaagtgtttagggcagagctaaaaaataaataaataaataaaaataaaataaaataaaaggaaaggaaaaaggtgagggaaattcctgatgattttgctaaagttaaaattccatagatttggctggctttatttcttttttttttttttttttttttttacatcatcaatttagaattctataaagaaagaatgacatcaaggaaaatcattggcctaggggaagagagcccgtaggcgtttaggtgttataaatatggaggcagagaacactggaggatcaggaagactcgcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtggggtaagtgaagaagcttttttaaaacaaatgtattttcatcagaggagtcggcatacacacactctacaatttaactttgtaggaaagaaaaataatttagaaaaaatcatggccccacattttgtcaaggattcttacaagtgatattcaaatatctaatctaaaatgattatctagaaattggcacattctaagtgtgcagatgctgatgaggagcaggtattgatagacagcgcgttatgcgtcaaaggatgtctatcctttgctaaagtgttactctgactatgctgtaaaaagcaggaggtaagagcttaagaaagaggagtaaaagagataattctcatgagataaactctaaggattgatgctgtgctccaggtctctccagtgttttagatgtttcaggatgctatttattacagaatatggtgtacttggaaaacatacagtagtaatcattttcctgattaacctaatttctagacagagtttgcattcatgaatggccacagtacagatgcggacatccaaaggatggcattattactcacaagcatagtgctatgtgcagttatggcttgagggaagggaggggggaggtcgccctctgagacctgaaccttttggtgtggtttcaagcactaaccagcactatctaatggctatttcactgccttgtcaatgacataggaaaaaggtacctgagtggaaactgttttcagggcacctttaaagcctgggagcaaagggtggagggatgattttccttgtggacttaaaagtctttaccctctttgtcctatttttctttcttccagaccaaaagtaccgagttcaacaacaccgtctcttgtagcaatcgggtgagtagagagttcagtgctgctggctttctccagggagacgccaggcattttggagagggagtatcctgctacgtgcagaactccgagaggtgcctgggctccgggacgccgccgccgggggaaaggggacatctgggctgtcagagcggggctgcgcctagcttgggacaacacttctgttccaatttagggagaggaagtctctatccggaggaaaggcaaattgggaactgggacgagggaacgttgttaggggcaccacctgctggggtccggcgcctccgcgctcgggctcggaattttggcagcctccgccccctggagacttgggaggagcgagcgtgggtgacagtcttttcgcgacgagtgccctccgccaccctcgccacgcccctgctcccccgcggttggttcttccttgctctactcaaccctgacctcttctctctgactctcgacttgtgttccccgctcctccctgaccttcctcccctcccctttcactcaattctcaccaactctttctctctctggtgttttctccttttctcgtaaactttgccgcctatgagcagccacattgccttactgaaatccagagcctaaccttcaatcccaccgccggctgcgcgtcgctcgccaaagaaatgttcgccatgaaaactaaggctgccttagctatctggtgcccaggctattcggaaactcaggtaagcccgaagcctcagacgtttgctgtaccttggggctaacctcaaattaaactggggctttggtgcagaagtcgttctcttatttttatttaggttttatctttcgaagagcaaacgagccgggtaaaagtggtaggatgtcagttagacccacgttgatacccggaatcaaactcacctatttctacggttctgatactgttttggctgaattatggttctaaaccttagggcaatgtttcaagctatgatgagtgagacttctatatcagaatgttttgattgctggagcataagagtatggcctcttgttcttatcacttaattattgtgtgcttatttgctaaatgtataattacattatacataaaatctctatcctatgtttgcttaattgcttgtgtgggcgctattgctgtctctttacacatttttgcacatgtagttatctgcatttgaatgctcgtgtagcattaaatatggagtttatttcagtcagcaagtagaggatttatcttcatggtgacaagtttaaggaacagagagagacaagtgcagatatgtttgattgctccttattagcctagtggactttatatgtctacagtctaggtagatggacacgactgtcacaaaactgtcactttctagaggttgaggattgaagccatagcgctgatctgggttgagcttgaattagaaactcaataccagacagccatatgggaaacctatttggcttcatgccttcttatgaaggagaccctggcaaatctgcagatggctacaataaaattcatttaaataagagcacaaacaaaaagctagatcaagttcttggacagcatgtgagaaagggagagtttggagaaatttatttcagtccctcccaagcccaaatggagagtctaagactaataataatgattttgcaggtttttttaagatttgtgcttaataaccctgtgactttattaatttgcataccatgtgtctaggaggcccagtgtactactcaaaggtaattcagataaaggtatatactgcaatcctctttaaaataagccctcagatgtctgtgacacatctagacaatggggcaggggagggggaaggatggggagcaggagcatgcattttgggtccaaaaaatagactaggtttattgaatgatgtctataaacaggtataagatagctcttgcccatgaggaacttgtgatcttgtcagggaggtcttgaaatcagcaatttattcatttcatgttaagtgagagccaagttaaatgacacacactcttaagtactggaagagtttccaaaagcacctggaaaaggcacatgctagcacatagtaagcaggtgctttggagacacactgaaagatggatttgcatagagaaggcaattaaacctgctctcaacagttactaaagatagtgaaaagtaattttgactattgattcttatattctgcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaattagctttcagcttctgctatgaaaatctctatcttggttttagtggacagaatactaagggtgtgacacttagaggaccactggtgtttattctttaattacagaagggattcttaacttattttttggcatatcgcttttttcagtataggtgctttaaatgggaaatgagcaatagaccgttaatggaaatatctgtactgttaatgaccagcttctgagaagtctttctcacctcccctgcacacaccttactctagggcaaacctaactgtagtaggaagagaattgaaagtagaaaaaaaaaattaaaaccaatgacagcatctaaaccctgtttaaaaggcaaggatttttctacctgtaatgattcttctaacattcctatgctaagattttaccaaagaagaaaatgacagttcgggcagtcactgccatgatgaggtggtctgaaagaagattgtggaatctgggagaaactgctgagatcatattgcaaatccagctgtcaaagggttcagacccaggacagtacaattcgtgagcagatctcaagagccttgcacatctacgagatatatatttaaagttgtagataatgaatttctaatttattttgtgagcacttttggaaatatacatgctactttgtaatgaatacatttctgaataaagtaattctcaagtttgtttcattcatttatttatttagttagttagttagtttggttttttgagacagggtttctctgtgtagccctggctatcctggagctcactctgtagaccaggctggcctcgaactcagaaatctgccttcctctgcctcccgagtgctgggattaaaggcgtgcgccaccacacctggctttcaagttcgtttcttatgaatggcgttttaaatttggttgagcaattttcatgcgtacttttctaagggacatcacggttgtctacatctttatcgccactcaagccgacatcccatgggccacacttcctttgatctggtatcaaccctccctgcaggagaaaaggtcttcataagtagttgcctcttggacaaatgactggagtgcatttttttcaaatatttgcaccagtcactccctcccactgtgaatctttcttcacctcagaatagataacacaggtgaaaatgaacagtgggtgttaaattcattcctgcacacctctggtaaaacaccctacctcttgccctcagaatcttctgagcattgctagcaaaggcaaccttggctgcagagctcaggccaagtaagagtagatgtaaacagctaacctgctcctccaccctacacacactctaagaagagatgttcacttgaatactgttttgaaggttagaactaacccattaatgaaaagaaaagctgagtgtccccaaacctgtcttacttgttgggagcgaccctgttggaatgttaactgccttgtcagccataagtgcttacttacaaagtcttgaccttagtggaaaaatactagcttagttgagatttctgtgggaaaagttgaagcctttgtaggaaagtactacccccagttaagaacaaatagttgtgctcactttggcagcacatatactaaaattggaacgatacagagaagattagcatggcccctgcgcaaggatgacacgcaaattcgtgaagtgttccatattttttgaagctgggacgaaaggacggaccatctagtgattgccatatccagggatccatcccataatcagcttccaaacgcttgacacactagcaagattttgctgaaaggacccagatatagctgtctcctgtgagactatgccggggcctagcaaacacataagtggatgctcacagtcagctattggatggatcacagggcccccaatggaggagctagagaaagtacccaaggaactaaagggaactgcaaccctataggtggaacaacaatatgaactaaccagtacccgggagctcttgtctctagctgcatatgtatcaaaagttggcctagtcggccatcactggaaagagaggcccattggacttgcaaactttatatgccccagtacaggggaacaccagagccaaaaagggggagtgggtgggtaggggagtcgggggatgggtatgggggacttttgggatagcattggaaatgtaaacgaggaaaatacctaataaattttttttttaaaaagtaaaaaaaaaaaaaaaaaaaagaacaaatagctatagatcttgtggacaggtacctagcaacccattctgttctgttcctcctgctgaactttttacctagccagtatcctgcttttggaacaggtgcattcccccagaaacaaagcgattctgcatcgtccccctccacatatcctgcttctgtgggtataaaacctgcctgggaaaaataaaatttgttagtttgatcagaatctttgatttgctgttcgttttttgtgtttcttgccccgccccccttctctctgcaggtggttcctcagaccctgttcaactgtcccgcatcagggcattacttgtcaacaaagagctacttatgagcaccaagtaaatagttacaaagtgcccactgtgggccaactttcctgaggtgaagtctgtgttaaacccatagttacaaaagtaagtaagacagagctcatatccagagaagctcagagtggaactggataatcagttgtctgtagtccttaacaaattggccagtgagtgttcctttgatttgagtaaaatcaagacaggcacactttcaaaaatcttcctctaaattccttacccagagcttttaagcaccaccctaagaaaactccactgggtctagaaaaggcagcaatcatcaattctttgaatagaagtgtggaggcctgatattttaaatgtattaactctgccttactacaaattcattctcccttttactaaatcatgataaaaggtattatagcatttttcttaatccctttagacccaattgccctaaaagtgacttctacccatttggtagagttcataggacagagtaccaaaggaaaggagtgctctgaggaggagaccattagaagataactcctgttattgaggacagcaataccaagcacatgccttaagaaaactgcactggagagatggggaaacatctggacaacaagagggactagtgtccattgctcactgcaagccagggaaatgagctgtgctcaccaggcagaatggaaaattctgtaacccatcaggctataagtagggtactgtgctgactagtttaatggcaacttgacacaaactagaaacatcagagaggaggaaacctcagctgagaaaatgcctttataatattcaggcatatggcattttcttaattagtgatcaatatgggaagggtcaacccattatgggtggggtcatccctgggctggttctaaaaaagcaggctgagaaagccatgggaagcaagcagcttcccatcatggcctcatattagctcctgccttcaggttcctgccctgcttgagtttctgtccttactttctttgatgatgaagagtgatgtgaaaatgtaggccaaataaaccctttcatcctcaacttgcttttttgtcatggtatttcatcccaaatatagaaaccccaagacatgtgcttaaaaacatcttacctgtgcatggaagtatcgttagaccaaggctaatggctgcaacgatctaacttaatgaatttaaaaaaaaataatacttaaaagaatcggttcctaagtaacttagctgtatttcacaacaaaccacaagggtgtttatgaagtaaagaatgtctcacacacatgcgaatgtatccactcaaatatatatatatataattaaaataaatctttaaagaatgaaagaaaaaagaaaacaaaaggggagagagggggaaggagtaagagaggatcttgaggacagaagagctgtaagaactattgtgtcctgttagggaaggtggcacaccttttatctagagtcagaaagcaggcaaatctttatgaagacgaacttcatctatacagtttcaggccagccaagctatacagtgagagcatgtctcaaaaataaggaggaaaatatggtgctgtagtataaaaagtaccactaactcaaaactaacatagaaggtagaattaataagtgaaacattaaattaattattataatgttgagaacaagcaaaagaaacttatcctaagttaaccattcccttttcagactccttttaattgtagtgagaaactaaaatcaaaatcccaggccctaggggagcttggaaattcctaacagctgaacagtttctattttaaggaaacagttgtccaagtccagatagctcagggacaacttctccatcttgctagtaagatcaaactaagttcaggtttccaggcccagaaacctacttctatccttattgatagaaactcccttgttcaacttcttatgtcaacatatgattggacaatgttatagtctaccctgctccccctcacttcacagttttgattccattctttaaataggctgtacagtgtcccttcagagttgcagctcagcacccaagtctgttctttggccctaactagtagacacttaattacaagaaaattttgccatctgcatggtgtttgaattatgttgtatttaagcagaccccacaacaataactcaagatatttaggaaacatagaagatacaagcacagattctagatatgaaaattatgtgtaaaataaatacacagtgaatagttttaattgggggttgggcattaaaatatttgaactagaccaatacccacccaaatgctacagcctggatgctccccaggagatccaaattgaccaaggacacaagtgacaatttcacccaaatacccatgccagctggaacacccacacagcccagctgacatgggacctacacccccaactctccaccctctatcccacccctctgagatccaccttccttctgatccaattcctcatccagaccaggtcagagacctagctgatacctgcccaaactctgcagcctgatcttcccaggaggtcagcagtaaccaagggcacaggaggtccacactaaccaaagacaaca

SEQ ID NO: 4 - DNA, 5789 bp, Mus musculus
gtgaaatgtatctagggccgctccccacccacccgttcctttatgctgttaagagatccaagtgagtcaagcccctgccccaactccctgagcccagaaggaagagaaatcagaggtctgctattcagtatctctaccactgccagggaacctgggacaattgagacagacaggtaccagcagagtggagctgctgggccagcacccagggaggggacagcacagagtgactatcaagagcccaggcagcagttaagagcatcaactgctcttctgaaggtcctgagttcaattcccagcaaccacgtggtggctcacaaccaccactaaggagatctgactccctcttctggagtgtctgatgatagctacagtgtacttacatataataataaataaatcttttttttaaaaaaagtatacacttaaaaaaaaaaagcccaggcaagttcccccccaccccgccccgctctgttcccccctctccccaggtcttctctaaaactcaatcccttgccagcatttcgaggctccaccgaaagcctgtctggatatctgatccaccatggaaaaggtcagttctcaggtgagctattgcaagagaaggctttccctcattccaaatgagagtccaaccccaccccccacccccgagtcacaagggaggcaggacagatgttgccatgggctgggatctgaaaatcagatctggactgcagttaagttcttccagagtggctaagcggtgtggacagccttcatttacacaacgaatatgtacctggccagtggcataagccagatcatgctagactccgtgagcctagactaaatagccaaaccaggccacgggccagccaatagcctagtaaggagcacagggcagacattagggttccttgggggctccatggctgttttcttaatagaaatttagaggggtgtgtgtgtgaagggggctgggggggggttgggaatcagtagacgtgggaaaaaagggtgtcacatacttccagcagctctgggctattaatggcaggaagaaaaggcccaccaggcttaacgatcttggaaccctgtgcaccgctgcctggcaccctgggcaggtcttctctagaaagtaaggtacctacactggctgggctctcaggtccctcggtttaagagagttataggcctatgtgcacacacgctggaactaggctacccagccccagcccagaagcccccactcaccaggacctgggtttacacacgcccacccttccgtgagagaggtcccagaggagaggacagattcagggccagctgaactctctccagtgtcttgtggtgtgcgccctggtgtgtggcgtgggtgggcttgttactgtggaagcttctttttaaaaagtcacagagtggagcaggccctcagtctctgccaagtgggatgcctggccagacgctggctgcatctgctaaccacctctgggtatcctggctgggtgcactgtcaatccctggcgcctcctctttgcaaatctgacacccagctgtccacagctctctgcctcaacgtccacggcaggtcaaccaagtcagctctgcctcgggctctcggaggtgggcctgactgatggctagccactgtctctgctgcccccctttcggccagcaagcgatcctaatccgcaatcccctctgagagccaggcttccgaagaaaggtggaggccgggttccgggcctgcagctctcacgtgctttcccggccaccccctcccgccctgcgtcgaggcggccaagcctgttcctcttcccccccgtcgcgattgcgacaggccggcctctgctcccagggctccctgcccccgcccccggccggctcgctccactcccacttcctgagcccggcgctggagccctggaggccaggcccggccgctcccggcccccgggggcacgtcggcccagccgccaggcttgggaagtcgtggccaacgctgctcaggacgtccgggcttcccaccttcctcctaggactcacccgtctggtcagccgagccgaaaggccgccatgctcctgccttgggccacctctgcccccggcctggcctgggggcctctggtgctgggcctcttcgggctcctggcagcatcgcagccccaggcggtgaggaaggggcctggtaggagtgggcgagggtgggcaagagggatctgggcagccgtcgctccattccctctgccctcccaagctgacccctgactaattcttctctcctcttctccatctccctttgaaggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaggaaactgggatcgctgctcaagaggcgtccgcagggagagggacccaatcctgtagctggaagctgggagcctccgaaggcccatccatacttccctgacttggtacagccactgctacccatttctggagatgtttccccagtatccactgggctccccgcagccccagttttggaggcaggggtgccgcaacagcagagtcctctggacctgaccagggagccgcagttggaacccggggagcagagccaggtggcccacggtaccaatggcattcatgtcaccggcgggtctatgactatcactggcaacatctacatctacaatggaccagtactggggggaccaccgggtcctggagacctcccagctacccccgaacctccataccccattcccgaagagggggaccctggccctcccgggctctctacaccccaccaggaagatggcaaggcttggcacctagcggagacagagcactgtggtgccacaccctctaacaggggcccaaggaaccaatttatcacccatgactgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacagtggctcaagtggcttgggtggtagaggtgagccagaatgagccagcactgctaaaactagccaggaaggagagtctacgaagcattagcattgtcccacggacactgagatttgaagaggtagcggcatgtagccatgaagacaggatggggacaaagagaccaaggagaggctccgaggcatgcagcaagcagaggcagcggacgcagagatggacttcttgtctcctgataaccctctttccccattcgcctcataggcgagtttgtctttgcggtatgcagccgcagccaagacacggtttgcaagacttgcccccataattcctataatgaacactggaaccatctctccacctgccagctgtgccgcccctgtgacattggtaagtggggactcatctggatctgcatgatgggtacgactgggagggccagctcctctctgactcttccctctccctgacagtgctgggctttgaggaggttgccccttgcaccagcgatcggaaagccgagtgccgctgtcagccggggatgtcctgtgtgtatctggacaatgagtgtgtgcactgtgaggaggagcggcttgtactctgccagcctggcacagaagccgaggtcacaggtcagaggtcactgagggcagccagtaaagggaggctgggcatcaagggcaaggaacgtgatactgtgcgcatggtgcttctccccactggtactgtgagtgtggtacctctgcccactgggagaaccataaagaatctatcagtccttgaaaaaggctcacaggagggggtctgccaagacatgaactggtatgaggagcttagaaggtagctccctcctgtcagccctggggaagcttgggcaaaacggcaggctgcaaagccaagcttgggaaaggtagcaactacagagcagaatggttggcaaagaggggacgtaaaggaaggccaccgagtcctcacacttacccactcacccccactggcctgcctttcttttgccagatgaaattatggatactgacgtcaactgtgtcccctgtaagccgggacacttccagaacacttcctcccctcgagcccgctgtcaaccccataccaggtgagagggcccttcccccactcacctccaggaaacccaagggttgtcatctcctccatccttgacttccggccatcccgaccatgtgttcctggagccagtcaccaaggggagcagggagaagctcacagtcttgtttctccacagatgtgagatccagggcctggtggaggcagctccaggtacctcctactcggataccatctgtaaaaatcccccagagccaggtaagacaccgggctgaggaacacaaggcagggtcggtctgggaagatgcctcagcccccctcatccacagaaacagggaacagtgcatctttcttcccagggttagacaaagtcagaaacatttcttctgaagaaatcagaaggaggtagcgtgtagttccatggttagaacgcttgcttgggatacataagaccctgagtttggaccaaaagaaaaaaaacgaaaacttggaaaggcaggtgtggtggtgcacccttgtaatccccagcgcttgaaaggctgcggcagaagaatcaagagtttgaggctagccttggctacagagtgagcctgtctccatagagggcctggagattagaacatccctagactcttttcttacactttcaaaattatacatattatgccaggaaacattcctgtgctgtgacgtaattctaaccggcttcatcactatgcttggatgtgattccgtcatagccttccttcactaattgaatacctcgttgttcacttacacacatctgttggagacatgctcccccactgggctctttctaggttttcttgtttcttggtttctgtcttcgaggaaacccactagtttcccagcctggtggttgactataagttcttctgatgactctaatcgctactaattggcagaatgtagtaacatttttgagtgaccagacttttgtaattatagcttccacatcctgagaacaactctgaacctct

SEQ ID NO: 5 - DNA, 20 bp, Artificial Sequence
  Synthetic
aagtgcagctcgccacccca

SEQ ID NO: 6 - DNA, 20 bp, Artificial Sequence
  Synthetic
aggaacttcatagcggtttc

SEQ ID NO: 7 - DNA, 20 bp, Artificial Sequence
  Synthetic
atgcttaggcataacgcact

SEQ ID NO: 8 - DNA, 20 bp, Artificial Sequence
  Synthetic
ccacgttcaggcgacagcat

SEQ ID NO: 9 - DNA, 20 bp, Artificial Sequence
  Synthetic
ttattctggagattgcatga

SEQ ID NO: 10 - DNA, 20 bp, Artificial Sequence
  Synthetic
gctcggctgaccagaccggg

SEQ ID NO: 11 - DNA, 20 bp, Artificial Sequence
  Synthetic
gagccactgttctcacctgg

SEQ ID NO: 12 - DNA, 22 bp, Artificial Sequence
  Synthetic
ggtaccagcagagttggatagc

SEQ ID NO: 13 - DNA, 22 bp, Artificial Sequence
  Synthetic
atcccttacacagaagctggag

SEQ ID NO: 14 - DNA, 23 bp, Artificial Sequence
  Synthetic
catctcctgtgggaccattcttc

SEQ ID NO: 15 - DNA, 22 bp, Artificial Sequence
  Synthetic
agtgcaggttatctcactgtgg

SEQ ID NO: 16 - DNA, 22 bp, Artificial Sequence
  Synthetic
ttggaactgaacccaagtgtgc

SEQ ID NO: 17 - DNA, 20 bp, Artificial Sequence
  Synthetic
ggctgtcctcagacccaatc

SEQ ID NO: 18 - DNA, 24 bp, Artificial Sequence
  Synthetic
gaagtttgttgctatggaagggtc

SEQ ID NO: 19 - DNA, 20 bp, Artificial Sequence
  Synthetic
agcgcaacgcaattaatgtg

SEQ ID NO: 20 - DNA, 22 bp, Artificial Sequence
  Synthetic
ccttctcgtgtgaataagctgc

SEQ ID NO: 21 - DNA, 23 bp, Artificial Sequence
  Synthetic
ctcatcagcatctgcacacttag

SEQ ID NO: 22 - DNA, 21 bp, Artificial Sequence
  Synthetic
cagggaggtcttgaaatcagc

SEQ ID NO: 23 - DNA, 21 bp, Artificial Sequence
  Synthetic
ccaggctgtagcatttgggtg

SEQ ID NO: 24 - DNA, 23 bp, Artificial Sequence
  Synthetic
gtgaaatgtatctagggccgctc

SEQ ID NO: 25 - DNA, 20 bp, Artificial Sequence
  Synthetic
tgctctgtctccgctaggtg

SEQ ID NO: 26 - DNA, 23 bp, Artificial Sequence
  Synthetic
agaggttcagagttgttctcagg

SEQ ID NO: 27 - DNA, 20 bp, Artificial Sequence
  Synthetic
atgcgtcggagaaccagacc

(19) United States
(12) Patent Application Publication
(10) Pub. No.: US 2023/0340524 A1
(43) Pub. Date: Oct. 26, 2023

(54) TRANSGENIC MOUSE MODELS SUPPORTING HUMAN INNATE IMMUNE FUNCTION

(71) Applicant: The Jackson Laboratory, Bar Harbor, ME (US)

(72) Inventors: Anna Karolina Palucka, Avon, CT (US); Chun l. Yu, New Britain, CT (US); Jacques Banchereau, Farmington, CT (US); Richard Maser, Bar Harbor, ME (US)

(73) Assignee: The Jackson Laboratory, Bar Harbor, ME (US)

(21) Appl. No.: 18/015,042

(22) PCT Filed: Jul. 7, 2021
(86) PCT No.: PCT/US21/40678

§ 371 Date: Jan. 6, 2023

Related U.S. Application Data
(60) Provisional application No. 63/049,175, filed on Jul. 8, 2020.

(51) Int. Cl. C12N 15/85; A01K 67/027; C12N 9/22; C12N 15/11; C07K 14/52; C07K 14/54; C07K 14/705; C07K 14/535

(52) U.S. Cl. CPC C12N 15/8509; A01K 67/027; C12N 9/22; C12N 15/11; C07K 14/52; C07K 14/5412; C07K 14/70578; C07K 14/535; C12N 2015/8536; A01K 2267/0362; A01K 2227/105; A01K 2217/07; C12N 2310/20

(57) ABSTRACT

The present disclosure provides immunodeficient NOD.Cg-Prkdc^{scid} Il2rg^{tm}^{_{1Wjl}}/SzJ (NSG™) mouse models that comprise an inactivated mouse Flt3 allele and, in some models, additional genetic modifications. These mouse models useful, for example, for superior engraftment of diverse hematopoietic lineages and for immune-oncology, immunology and infectious disease studies.

### RELATED APPLICATION

This application claims the benefit under 35 U.S.C. § 119(e) of U.S. provisional application No. 63/049,175, filed Jul. 8, 2020, which is incorporated by reference herein in its entirety.

### BACKGROUND

Mouse models have been used extensively to study human diseases in vivo to circumvent the complexity dealing with human patients. Nevertheless, murine models often inadequately recapitulate the human disease partly due to important differences between mouse and human immune systems (Hagai et al., 2018; Kanazawa, 2007; Mestas & Hughes, 2004; Williams, Flavell, & Eisenbarth, 2010). Thus, humanized mice, defined as mice with human immune system, could be an attractive alternative (Shultz, Brehm, Garcia-Martinez, & Greiner, 2012; Theocharides, Rongvaux, Fritsch, Flavell, & Manz, 2016; Victor Garcia, 2016; Zhang & Su, 2012). To this end immunodeficient mice lacking common gamma chain (γc) like NOD-SCID-Il 2γc^{-/-} (NSG), or BALB/c-Rag2^{-/-}-γc^{-/-} (BRG) (Matsumura et al., 2003; Traggiai et al., 2004) can be humanized by transplantation of human CD34⁺ hematopoietic progenitor cells (HPCs). Based on the sources of T cells, the model can be further categorized into two types: (1) a model in which mature T cells are isolated from the donor of HPCs and adoptively transferred (Aspord et al., 2007; Pedroza-Gonzalez et al., 2011; Wu et al., 2014; Wu et al., 2018; Yu et al., 2008); in this case the T cells have been selected in human thymus; and (2) a model in which endogenous T cells are de novo generated from human CD34⁺ HPCs (Matsumura et al., 2003; Traggiai et al., 2004); in which case human T cells are selected in mouse thymus.

### SUMMARY

The present disclosure provides multiple improved immunodeficient mice generated primarily using CRISPR technology for one-step generation of animals carrying mutations (Table 1) (Wang et al., 2013). These models were generated to address limitations of the models discussed above. The biggest limitation of the first model in which mature T cells are isolated from the donor of HPCs and adoptively transferred is graft-versus-host disease; the biggest limitation of the second model in which endogenous T cells are de novo generated from human CD34⁺ HPCs is a limited number of T cells able to recognize human major histocompatibility complex (MHC). Furthermore, substantial limitations remain that hamper the use of humanized mice for advanced in vivo studies including: 1) incomplete development of a full range of hematopoietic lineages like neutrophils, erythrocytes, Langerhans cells (Shultz et al., 2012); 2) limited long-term engraftment, especially of myeloid cells, which leads to an imbalance between myeloid and lymphoid lineages over time (Audige et al., 2017); 3) insufficient support of the engraftment of adult CD34+ HPCs derived from blood or bone marrow, which hampers the feasibility of constructing fully autologous models where the tumor and the immune system are from the same patient (Saito et al., 2016); 4) insufficient colonization of non-lymphoid tissues (for example mucosal barriers) with both myeloid and lymphoid cells (Herndler-Brandstetter et al., 2017; Rongvaux et al., 2014); and last but not least maturation of human adaptive immunity in the context of mouse major histocompatibility complex (MHC).

The strategy used herein to improve humanized mice is based, at least in part, on the concept that improved development of human myeloid cells and specifically of human dendritic cells (DCs) will improve adaptive immunity. We approached this in a stepwise manner. Because DCs are critical for proper immune homeostasis and for the generation of adaptive immunity (Banchereau & Steinman, 1998), we started by creating the mouse Fms Related Receptor Tyrosine Kinase 3 (Flt3) knockout (KO) models to produce a more permissible environment for human DC development by the inhibition of mouse DCs. We then made human Interleukin 6 (IL6) knockin (KI), human lymphotoxin beta receptor (LTBR) KI and human thymic stromal lymphopoietin (TSLP) KI in the mouse Flt3 KO model and crossed existing NSG mice with transgenic (Tg) expression of human Stem Cell Factor (SCF), Granulocyte Macrophage-Colony Stimulating Factor (GM-CSF) and Interleukin 3 (IL3) (NSG-SGM3, SGM3) (Nicolini, Cashman, Hogge, Humphries, & Eaves, 2004; Wunderlich et al., 2010) in the mouse Flt3 KO model.

The mouse Flt3 KO models provided herein create space for human DCs and, by making the receptor ligand Flt3L available to human cells, improve the development of human myeloid cells upon transplant with human CD34+ HPCs. Moreover, the Flt3 KO models with additional human KI or Tg gene expression engrafted with human HPCs can generate human vaccine-specific antibodies including neutralizing antibodies against influenza virus. Overall, the strains of the present invention address existing limitation of humanized mouse model for translational immunology/immune-oncology studies.

Thus, some aspects of the present disclosure provide a non-obese diabetic (NOD) mouse comprising an inactivated mouse Prkdc allele, an inactivated mouse IL2rg allele, and an inactivated mouse Flt3 allele. Further aspects of the present disclosure provide an NSG™ mouse comprising an inactivated mouse Flt3 allele. Further aspects of the present disclosure provide an NSG™ mouse comprising an inactivated mouse Flt3 allele.

Also provided herein are methods of producing an NOD mouse comprising an inactivated mouse Prkdc allele, an inactivated mouse IL2rg allele, and an inactivated mouse Flt3 allele, methods of using the mouse as a model system, and methods of propagating the mouse.

Some aspects of the present disclosure provide an NOD mouse comprising an inactivated mouse Prkdc allele, an inactivated mouse IL2rg allele, an inactivated mouse Flt3 allele, and a nucleic acid encoding human TSLP. Further aspects of the present disclosure provide an NSG™ mouse comprising an inactivated mouse Flt3 allele, and a nucleic acid encoding human TSLP.

Also provided herein are methods of producing an NOD mouse comprising an inactivated mouse Prkdc allele, an inactivated mouse IL2rg allele, an inactivated mouse Flt3 allele, and a nucleic acid encoding human TSLP, methods of using the mouse as a model system, and methods of propagating the mouse.

Some aspects of the present disclosure provide an NOD mouse comprising an inactivated mouse Prkdc allele, an inactivated mouse IL2rg allele, an inactivated mouse Flt3 allele, and a nucleic acid encoding human IL6. Further aspects of the present disclosure provide an NSG™ mouse comprising an inactivated mouse Flt3 allele, and a nucleic acid encoding human IL6.

Also provided herein are methods of producing an NOD mouse comprising an inactivated mouse Prkdc allele, an inactivated mouse IL2rg allele, an inactivated mouse Flt3 allele, and a nucleic acid encoding human IL6, methods of using the mouse as a model system, and methods of propagating the mouse.

Some aspects of the present disclosure provide an NOD mouse comprising an inactivated mouse Prkdc allele, an inactivated mouse IL2rg allele, an inactivated mouse Flt3 allele, and a nucleic acid encoding human LTBR. Further aspects of the present disclosure provide an NSG™ mouse comprising an inactivated mouse Flt3 allele, and a nucleic acid encoding human LTBR.

Also provided herein are methods of producing an NOD mouse comprising an inactivated mouse Prkdc allele, an inactivated mouse IL2rg allele, an inactivated mouse Flt3 allele, and a nucleic acid encoding human LTBR, methods of using the mouse as a model system, and methods of propagating the mouse.

Some aspects of the present disclosure provide an NOD mouse comprising an inactivated mouse Prkdc allele, an inactivated mouse IL2rg allele, an inactivated mouse Flt3 allele, a nucleic acid encoding human IL3; a nucleic acid encoding human GM-CSF; and a nucleic acid encoding human SCF. Further aspects of the present disclosure provide an NSG™ mouse comprising an inactivated mouse Flt3 allele, and a nucleic acid encoding human IL3, a nucleic acid encoding human GM-CSF, and a nucleic acid encoding human SF.

Also provided herein are methods of producing an NOD mouse comprising an inactivated mouse Prkdc allele, an inactivated mouse IL2rg allele, an inactivated mouse Flt3 allele, a nucleic acid encoding human IL3; a nucleic acid encoding human GM-CSF; and a nucleic acid encoding human SCF, methods of using the mouse as a model system, and methods of propagating the mouse.

Further aspects of the present disclosure provide cells obtained from any one of the mouse models described herein.

### BRIEF DESCRIPTION OF THE DRAWINGS

FIGS. 1A-E depict mouse Flt3 knockout in NSG mice via CRISPR/cas. FIG. 1A depicts a schematic showing a chromosomal deletion at the exon 3 of Flt3 in NSG mice with a Flt3 knockout (NSGF). FIG. 1B depicts F1 littermates tail tipped to detect the mouse Flt3 wildtype allele (799 bp) and mutant allele (363 bp) by PCR. FIG. 1C depicts mouse Flt3 protein expression analyzed on bone marrow mCD45+ cells in 8-10 week old mice by FACS. FIG. 1D is a graph of summary data from FIG. 1C from n=7 mice. Data points symbols are square: male; round: female. FIG. 1E is a graph depicting 8-10 week old mice analyzed for mouse Flt3L production in the plasma by ELISA. Data points symbols are square: male; round: female.

FIGS. 2A-2C depict mouse Flt3 knockout led to a decrease in murine dendritic cells (DCs). FIG. 2A depicts single cell suspension of bone marrow, spleen, and lungs of mice at 8-10 weeks of age stained with specific antibodies and analyzed by flow cytometry. pDCs were gated as DAPI−, mCD45+, mCD3/19−, F4/80−, and Gr1− with expression of MHC class II and PDCA-1. PDCA-1− cells were further gated for MHC class II+ and mCD11c+ for cDCs. cDCs were divided into mCD11b+ or mCD8+ subsets in the bone marrow and spleen and mCD103+ subsets in the lungs. FIG. 2B is a graph of summary data from FIG. 2A from n=7 mice. FIG. 2C depicts localization of mouse MHC class II (IAg7) and DAPI in the spleen of NSG or NSGF mice at 8-10 weeks of age. Scale bar=100 m.

FIGS. 3A-3F depict improved human engraftment in humanized NSGF mice. FIG. 3A is a schematic depicting the construction of humanized mice. Mice were sublethal irradiated at 4 weeks and engrafted with human CD34+ HPCs, bled monthly, and analyzed at 16 weeks post HPC transplant. FIG. 3B is a graph depicting kinetics of human engraftment in the blood by the percentage of hCD45+ cells in hNSG or hNSGF mice after transplant of 1×10⁵ fetal liver HPCs. FIG. 3C is a graph depicting the percentages of different human immune cells analyzed in the blood by FACS in FIG. 3B. FIG. 3D depicts localization of human MHC class II (HLA-DR, green), mouse MHC class II (IAg7) and DAPI in the spleen and gut of hNSG or hNSGF mice at 15 weeks after fetal liver HPC transplant. Scale bar=50 μm. FIG. 3E are graphs depicting human engraftment as measured in the blood by percentage, the absolute number of hCD45+ cells, and the percentage of human CD33+, CD19+, and CD3+ cells at 12 weeks after transplant at either newborn (NB) or week 4 (W4) with 1×10⁵ cord blood (CB) HPCs. FIG. 3F are graphs depicting human engraftment at 12 weeks after transplant at week 4 with 1×10⁵ bone marrow (BM) HPCs.

FIGS. 4A-4J depict human IL6 knockin in NSGF mice via CRISPR/Cas. FIG. 4A depicts potential founder mice that were selected by positive PCR assay targeting 5′ and 3′ junctions and full length of human IL6-knockin sequence and negative for plasmid backbone. FIG. 4B is a graph depicting human IL-6 production in the plasma of NSGF mice with different IL6 alleles treated with 10 μg LPS i.p. for 2 hours. FIG. 4C depicts human engraftment in the blood by the percentage (left panels) and absolute number (right panels) of hCD45+ cells in hNSG or hNSGF6 mice after transplant with 1×10⁴, 3×10⁴, 1×10⁵ HPCs from cord blood for 12 weeks. n=2-3 mice from one donor. FIG. 4D depicts human engraftment in the blood by the percentage (left panels) and absolute number (right panels) of hCD45+ cells in hNSG or hNSGF6 mice after transplant of 1×10⁵ bone marrow HPCs for 12 weeks. n=5 from one bone marrow donor. FIG. 4E depicts human monocyte subsets in the spleen and lungs of humanized mice analyzed at 20 weeks by FACS. n=4 mice from two cord blood donors. Representative FACS plots from one mouse per strain were shown. FIG. 4F depicts the summary of the absolute number of CD14⁺ cells in the spleen (left panel) and lungs (right panel). FIG. 4G depicts the summary of the absolute number of CD14⁺ cell subsets in the spleen and lungs. FIG. 4H depicts human CXCR5+PD1⁺CD4⁺ Tfh cells in the spleen of humanized mice that were analyzed at 20 weeks by FACS. FIG. 4I depicts the summary of the absolute number of CXCR5+PD1⁺CD4⁺ Tfh cells in the spleen. n=4 mice from two cord blood donors. FIG. 4J depicts total antibody in the serum of humanized mice analyzed at 16 weeks by ELISA. Summary of total IgM (left panel), IgG (middle panel) and IgA (right panel). n=9-24 mice from two cord blood donors.

FIGS. 5A-5C depict human TSLP knockin in NSGF mice via CRISPR/Cas. FIG. 5A depicts potential founder mice that were selected by positive PCR assay targeting 5′ and 3′ junctions of human TSLP-knockin sequence. FIG. 5B is a graph depicting human TSLP protein production in the lungs of mice treated with PMA/IONO for 18 hours. FIG. 5C are graphs depicting human engraftment measured in the blood by percentage of human CD33+, CD19+, CD3+ cells at 12 weeks after transplant at either newborn (NB) or week-4 (W4) with 1×10⁵ cord blood (CB) HPCs.

FIGS. 6A-6C depict human LTBR knockin in NSGF mice via CRISPR/Cas. FIG. 6A is a schematic depicting knockin strategy targeting the ATG and STOP codons of mouse Ltbr using a plasmid donor insert human LTBR coding sequence (including intron 1) followed by a bGHpA STOP cassette. FIG. 6B is a graph depicting mouse and human LTBR expression analyzed on bone marrow mCD45+ cells at 6-8 weeks old mice by FACS. Summary data is from n=5 mice. FIG. 6C are graphs depicting human engraftment measured in the blood by the percentage and the absolute number of hCD45+ cells as well as the percentage of human CD33+, CD19+, and CD3+ cells in hCD45+ cells at 12 weeks after transplant at either newborn (NB) or week-4 (W4) with 1×10⁵ cord blood (CB) HPCs.

FIGS. 7A-7B depict superior human engraftment in SGM3F mice. FIG. 7A are graphs depicting human engraftment in the blood of mice (n=6-18, 4 weeks old) transplanted with cord blood HPCs derived from five cord blood donors. Human engraftment in the blood of the mice was measured at 12 weeks after transplant and analyzed by the percentage of hCD45+ cells and the percentage of CD33+ or CD14+, CD19+, CD3+ cells by FACS. Statistically significant differences were determined using an ANOVA test. FIG. 7B are graphs depicting human engraftment in the blood of mice (n=6-18, 4 weeks old) transplanted with bone marrow HPCs. Human engraftment in the blood of the mice was measured at 12 weeks after transplant and analyzed by the percentage of hCD45+ cells and the percentage of CD33+ or CD14+, CD19+, CD3+ cells by FACS. Statistically significant differences were determined using an ANOVA test.

FIGS. 8A-8D depict expansion of human myeloid compartment in SGM3F mice. FIG. 8A are graphs depicting humanized mice (4 weeks old) transplanted with cord blood HPCs. Human myeloid subsets in the spleen of humanized mice were analyzed at 20 weeks by FACS. Summary of different myeloid cells in the bone marrow and spleen from mice (n=3). FIG. 8B are graphs depicting a summary of DC subsets. FIG. 8C are graphs depicting a summary of cDC subsets. FIG. 8D depicts localization of human HLA-DR, human CD3 and DAPI in the gut of humanized mice analyzed at 20 weeks. Scale bar=50 m.

FIGS. 9A-9D depict increased T cell differentiation in SGM3F mice. FIG. 9A depicts FACS analysis of humanized mice that were transplanted at 4 weeks old with cord blood HPCs. Human CD3+ thymocytes were analyzed by FACS for CD4 and CD8 subsets at 20 weeks after transplant. Results show pooled n=3 mice from one cord blood donor. FIG. 9B depicts localization of human T cells in the thymus of humanized mice analyzed at 20 weeks. Human HLA-DR and human CD3 in the upper panel vs. human CD4 and human CD8 in the lower panel. Scale bar=30 m. FIG. 9C is a graph depicting a summary of the CD4+ to CD8+ T cell ratio in the spleen. Statistically significant differences were determined using a Oneway ANOVA test. FIG. 9D are graphs depicting summaries of the CD4+ and CD8+ T cell subsets including CD45+ CCR7+ naïve T cells (Tn), CD45RA-CCR7+ memory T cells (Tm), and CCR7− effector T cells (Teff) in the spleen.

FIGS. 10A-10C depicts specific antibody response in SGM3F mice. FIG. 10A are graphs depicting total antibodies in the plasma of mice 20 weeks after transplant as measured by ELISA. The humanized mice (n=3, 4 weeks old) were transplanted with cord blood HPCs (from one cord blood donor). FIG. 10B depicts humanized mice (n=3, 4 weeks old) transplanted with cord blood HPCs (from one cord blood donor), were vaccinated 3 times with KLH at 3-week intervals after 14 weeks, and measured KLH-specific IgG analyzed by ELISA. FIG. 10C depicts humanized mice (n=6-9, 4 weeks old) were transplanted with cord blood HPCs (from 2 cord blood donors), were vaccinated 2 times with Fluzone at 3-week intervals, and measured Fluzone-specific IgG were analyzed by ELISA. Neutralizing antibody to influenza A/Cal9 virus were measured by hemagglutination assay.

### DETAILED DESCRIPTION

The present disclosure provides immunodeficient NOD.Cg-Prkdc^{scid} Il2rg^{tm1Wjl}/SzJ (NSG™) mouse models that comprise an inactivated mouse Flt3 allele and, in some models, additional genetic modifications. The mouse models provided herein are useful, for example, for superior engraftment of diverse hematopoietic lineages and for immune-oncology, immunology and infectious disease studies.

Flt3 is a receptor important for development of the dendritic cells and monocytic lineages. Flt3L-Flt3 signaling is important for the development of various DC and monocytic lineages (Ding et al., 2014; Ginhoux et al., 2009; McKenna et al., 2000; Waskow et al., 2008) and it's role is further supported by the increase of circulating conventional (c)DCs and plasmacytoid (p)DCs after the administration of Flt3L in vivo in mice and humans (Karsunky, Merad, Cozzio, Weissman, & Manz, 2003; Maraskovsky et al., 1996; Pulendran et al., 2000). Knocking-out mouse Flt3 can lead to: (1) decrease in murine DCs and other myeloid cells; and (2) increase in the availability of mouse Flt3L (which can act via human receptor) to human cells, thereby improving the long-term development of human myeloid cells upon transplant with human CD34+ HPCs. The present disclosure, in some embodiments, uses a CRISPR/Cas system to generate Flt3 KO mice in an NSG™ background.

Thus, in some aspects, the present disclosure provides mouse models having a NOD.Cg-Prkdc^{scid}Il2rg^{tm1Wjl}/SzJ (NSG™) background and further comprising an inactivated mouse Fit allele (referred to herein as NSGF mice). In some embodiments, the genotype of an NSGF mouse model is NSG™ Flt3^{em1Akp} (see Example 1 for an exemplary method of generating the NSG™ Flt3^{em1Akp} mouse).

Other aspects of the present disclosure provide mouse models having an NSG™ background and further comprising an inactivated mouse Flt3 allele and a nucleic acid encoding human IL6 in lieu of mouse Il6 (referred to herein as NSGF6 mice). In some embodiments, the genotype of an NSGF6 mouse model is NSG™ Flt3^{em1Akp} Il6^{e1m(IL6)Akp} (see Example 2 for an exemplary method of generating the NSG™ Flt3^{em1Akp} Il6^{em1(IL6)Akp} mouse).

Yet other aspects of the present disclosure provide mouse models having an NSG™ background and further comprising an inactivated mouse Flt3 allele and a nucleic acid encoding human TSLP in lieu of mouse Tslp (referred to herein as NSGFT mice). In some embodiments, the genotype of an NSGFT mouse model is NSG™ Flt3^{em1Akp} Tslp^{em3(TSLP)Akp} (see Example 3 for an exemplary method of generating the NSG™ Flt3^{em1Akp} Tslp^{em3(TSLP)Akp} mouse).

Still other aspects of the present disclosure provide mouse models having an NSG™ background and further comprising an inactivated mouse Flt3 allele and a nucleic acid encoding human LTBR in lieu of mouse Ltbr (referred to herein as NSGFL mice). In some embodiments, the genotype of an NSGFL mouse model is NSG™ Flt3^{em1Akp} Ltbr^{em1(LTBR)Akp} (see Example 4 for an exemplary method of generating the NSG™ Flt3^{em1Akp} Ltbr^{em1(LTBR)Akp} mouse).

Further aspects of the present disclosure provide mouse models having an NSG™ background and further comprising an inactivated mouse Flt3 allele and a nucleic acid encoding human IL3, GM-CSF, and SCF (referred to herein as SGM3F mice). In some embodiments, the genotype of an SGM3F mouse model is NSG™ Flt3^{em1Akp}-Tg(Hu-CMV-IL3, CSF2, KITLG)^{1Eav/MloySzJ} (see Example 5 for an exemplary method of generating the NSG™ Flt3^{em1Akp}-Tg(Hu-CMV-IL3, CSF2, KITLG)^{1Eav/MloySzJ} mouse).

### The NSG™ and NSGF Mouse Models

The NSG™ mouse is an immunodeficient mouse that lacks mature T cells, B cells, and natural killer (NK) cells, is deficient in multiple cytokine signaling pathways, and has many defects in innate immunity (see, e.g., (Shultz, Ishikawa, & Greiner, 2007; Shultz et al., 2005; Shultz et al., 1995), each of which is incorporated herein by reference). The NSG™ mouse, derived from the non-obese diabetic (NOD) mouse strain NOD/ShiLtJ (see, e.g., (Makino et al., 1980), which is incorporated herein by reference), include the Prkdc^{scid} mutation (also referred to as the “severe combined immunodeficiency” mutation or the “scid” mutation) and the Il2rg^{tm1Wjl} targeted mutation. The Prkdc^{scid} mutation is a loss-of-function mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; Shultz et al., 2005), each of which is incorporated herein by reference). A loss-of-function mutation, as is known in the art, results in a gene product with little or no function. By comparison, a null mutation results in a gene product with no function. An inactivated allele may be a loss-of-function allele or a null allele.

An inactivated allele is an allele that does not produce a detectable level of a functional gene product (e.g., a functional protein). In some embodiments, an inactivated allele is not transcribed. In some embodiments, an inactivated allele does not encode a functional protein. Thus, a mouse comprising an inactivated mouse Flt3 allele does not produce a detectable level of functional FLT3. In some embodiments, a mouse comprising an inactivated mouse Flt3 allele does not produce any functional FLT3.

The mouse models provide herein (e.g., the NSGF, NSGF6, NSGFT, NSGFL, or SGM3F mice, or any combination thereof) comprise a genomic modification that inactivates the mouse Flt3 allele. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome, relative to the corresponding wild-type nucleic acid (e.g., the naturally-occurring nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs). In some embodiments, CRISPR/Cas gene editing is used to inactivate the mouse Flt3 allele, as described elsewhere herein. In some embodiments, a genomic modification (e.g., a deletion or an indel) is in a (at least one) region of the mouse Flt3 allele selected from coding regions, non-coding regions, and regulatory regions. In some embodiments, the genomic modification (e.g., a deletion or an indel) is a coding region of the mouse Flt3 allele. For example, the genomic modification (e.g., a deletion or an indel) may be in exon 3, or it may span exon 3 of the mouse Flt3 allele. In some embodiments, the genomic modification is a genomic deletion. For example, the mouse Flt3 allele may comprise a genomic deletion of nucleotide sequences in exon 3. In some embodiments, the nucleotide sequence of SEQ ID NO: 1 has been deleted from an inactivated mouse Flt3 allele. In some embodiments, an inactivated mouse Flt3 allele comprises the nucleotide sequence of SEQ ID NO: 1.

In some embodiments, the mouse models provided herein (e.g., the NSGF, NSGF6, NSGFT, NSGFL, or SGM3F mice, or any combination thereof) do not express a detectable level of mouse FLT3. A detectable level of mouse FLT3 is any level of FLT3 protein detected using a standard protein detection assay, such as flow cytometry and/or an ELISA. In some embodiments, a mouse model (e.g., the NSGF, NSGF6, NSGFT, NSGFL, or SGM3F mice, or any combination thereof) expresses an undetectable level or a low level of mouse FLT3. For example, a mouse model may express less than 1,000 pg/ml mouse FLT3. In some embodiments, mouse model expresses less than 500 pg/ml mouse FLT3 or less than 100 pg/ml mouse FLT3. The mouse FLT3 receptor is also referred to as cluster of differentiation antigen CD135. Thus, in some embodiments, a mouse model (e.g., the NSGF, NSGF6, NSGFT, NSGFL, or SGM3F mice, or any combination thereof) does not comprise (there is an absence of) CD135+ multipotent progenitor cells.

Flt3 knockout mice, in some embodiments, are generated by CRISPR using Cas9 mRNA and a guide RNA (gRNA). In some embodiments, the gRNA (e.g., 5′-AAGTGCAGCTCGCCACCCCA-3′, SEQ ID NO: 5) targets exon 3 of mouse Flt3 of NSG™ mice (NOD.Cg-Prkdc^{scid} Il2rg^{tm1Wjl}; RRID:IMSR JAX:005557). The blastocysts derived from the injected embryos, in some embodiments, are transplanted into foster mothers and newborn pups are obtained. In some embodiments, mice carrying a null deletion are backcrossed to NSG™. F0 and F1 littermates may be tested for successful gene-knockout by PCR and Sanger sequencing, for example. For example, primers (5′-GGTACCAGCAGAGTTGGATAGC-3′, SEQ ID NO: 12) and (5′-ATCCCTTACACAGAAGCTGGAG-3′, SEQ ID NO: 13) may be used in a PCR reaction to detect the mouse Flt3 wildtype allele from mutant allele (Table 2). The WT allele yields a DNA fragment 799 bp in length, whereas the mutated allele generates a DNA fragment of 363 bp in length.

### Knockin Mouse Models

Knockin mouse models (KI mice) can be generated to modify a gene sequence, for example, by substituting the gene sequence with a transgene, or by adding a gene sequence that is not found within the locus. The NSGF6, NSGFT, NSGFL, and SGM3F mouse models provided herein include a knockin allele. They include an exogenous nucleic acid that has been introduced into the mouse genome.

A nucleic acid used as provided herein may be a DNA, an RNA, or a chimera of DNA and RNA. In some embodiments, a nucleic acid (e.g., DNA) comprises a gene encoding a particular protein of interest (e.g., IL6, TSLP, LTBR, IL3, GM-CSF, SCF, or any combination thereof). A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences). As is known in the art, a promoter sequence is a DNA sequence at which transcription of a gene begins. Promoter sequences are typically located directly upstream of (at the 5′ end of) a transcription initiation site. An exon is a region of a gene that codes for amino acids. An intron (and other non-coding DNA) is a region of a gene that does not code for amino acids.

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

Methods of producing a knockin mouse model are described elsewhere herein.

### The NSGF6 Mouse Models

The present disclosure provides mouse models having an NSG™ background and further comprising an inactivated mouse Flt3 allele and a nucleic acid encoding human IL6 in lieu of mouse Il6 (referred to herein as NSGF6 mice). In some embodiments, the genotype of an NSGF6 mouse model is NSG™ Flt3^{em1Akp} Il6^{em1(IL6)Akp} (see Example 2 for an exemplary method of generating the NSG™ Flt3^{em1Akp} Il6^{em1(IL6)A kp} mouse).

IL6 (e.g., NC_000007.1; chromosome:GRCh38:7:22725889-22732002) is a cytokine and growth factor that stimulates inflammation and the maturation of immune cells (e.g., B cells) by binding and activating the interleukin 6 receptor, alpha. IL6 is essential in HPC maintenance (Encabo, Mateu, Carbonell-Uberos, & Minana, 2003) and in the differentiation of activated B cells into antibody producing plasma cells (Jego et al., 2003; Nurieva et al., 2009). To improve the NSG-based humanized mice, human IL6 knockin mice were generated to replace the mouse ortholog in NSGF mice.

In some embodiments, the NSGF6 mice described herein comprise an inactivated mouse Flt3 allele and a nucleic acid encoding IL6. In some embodiments, the nucleic acid encodes human IL6. In some embodiments, the nucleic acid comprises a human IL6 transgene. In some embodiments, a transgene, such as a human IL6 transgene, is integrated into a mouse genome. In some embodiments, a human IL6 transgene comprises the nucleic acid sequence of SEQ ID NO: 2.

Human IL6 knockin mice, in some embodiments, are generated using a CRISPR/cas system. Cas9 mRNA, gRNAs targeting mouse Il6 and recombinant human IL6 DNA, for example, may be coinjected into fertilized NSGF oocytes (e.g., NOD.Cg-Prkdc^{scid} Il2rg^{tm1Wjl} Flt3^{em1Akp}). Human IL6, in some embodiments, is inserted into exon 1 and exon 5 via homologous recombination. In some embodiments, the resulting founders, carrying human IL6 are bred, for example, to NSGF mice for multiple (e.g., two generations), and are then interbred until all offspring are homozygous for the Il6 targeted mutation. Examples of primers that may be used for genotype by PCR reaction are listed in Table 2.

### The NSGFT Mouse Models

The present disclosure also provides mouse models having an NSG™ background and further comprising an inactivated mouse Flt3 allele and a nucleic acid encoding human TSLP in lieu of mouse Tslp (referred to herein as NSGFT mice). In some embodiments, the genotype of an NSGFT mouse model is NSG™ Flt3^{em1Akp}Tslp^{em3(TSLP)Akp} (see Example 3 for an exemplary method of generating the NSG™ Flt3^{em1Akp}Tslp^{em3(TSLP)Akp} mouse).

Thymic stromal lymphopoietin (TSLP) (e.g., NC_000005.10;

chromosome:GRCh38:5:111070080-111078026) is a species-specific cytokine and exhibits species-specific function (Hanabuchi, Watanabe, & Liu, 2012). Human TSLP induces proliferation of naïve T cells, drive Th2 differentiation, Tregs development (Hanabuchi et al., 2010; Ito et al., 2005; Lu et al., 2009). TSLP stimulates the production of immune cells (e.g., B cells and T cells) by binding and activating the heterodimeric receptor complex composed of the thymic stromal lymphopoietin receptor chain and the IL-7R alpha chain (see, e.g., (He & Geha, 2010)). TSLP is also important for the polarization of dendritic cells. In contrast to IL-7 which directly acts on CD4+ T cells, TSLP mediates T cell homeostasis indirectly through human DCs (Lu et al., 2009). To improve the T cell development and differentiation, human TSLP knockin mice were generated to replace mouse Tslp in NSGF mice.

In some embodiments, the NSGFT mice described herein comprise an inactivated mouse Flt3 allele and a nucleic acid encoding TSLP. In some embodiments, the nucleic acid encodes human TSLP. In some embodiments, the nucleic acid comprises a human TSLP transgene. In some embodiments, a transgene, such as a human TSLP transgene, is integrated into a mouse genome. In some embodiments, a human TSLP transgene comprises the nucleic acid sequence of SEQ ID NO: 3.

Human TSLP knockin mice, in some embodiments, are generated using a CRISPR/cas system. Cas9 mRNA, gRNAs targeting mouse Tslp and recombinant human TSLP DNA, for example, may be coinjected into fertilized NSGF oocytes (e.g., NOD.Cg-Prkdc^{scid} Il2rg^{tm1Wjl} Flt3^{em1Akp}). Human TSLP, in some embodiments, is inserted into exon 1 and exon 5 via homologous recombination. In some embodiments, the resulting founders, carrying human TSLP are bred, for example, to NOD.Cg-Prkdc^{scid} Il2rg^{tm1Wjl} Flt3^{em1Akp} mice, and are then interbred until all offspring are homozygous for the TSLP targeted mutation. Examples of primers that may be used for genotype by PCR reaction are listed in Table 2.

### The NSGFL Mouse Models

The present disclosure further provides mouse models having an NSG™ background and further comprising an inactivated mouse Flt3 allele and a nucleic acid encoding human LTBR in lieu of mouse Ltbr (referred to herein as NSGFL mice). In some embodiments, the genotype of an NSGFL mouse model is NSG™ Flt3^{em1Akp} Ltbr^{em1(LTBR)Akp} (see Example 4 for an exemplary method of generating the NSG™ Flt3^{em1Akp} Ltbr^{em1(LTBR)Akp} mouse).

Follicular dendritic cells (FDCs) are essential for the development of lymphoid follicles and B cell responses (Futterer, Mink, Luz, Kosco-Vilbois, & Pfeffer, 1998). PDGFRb⁺ Mfge8⁺ FDC precursors in the perivascular area of Rag2^{-/-}-γc^{-/-} mice could differentiated into mature FDCs upon the activation of lymphotoxin beta receptor (LTBR) (e.g., NC_000012.12; chromosome:GRCh38:12:6375160-6391571) through lymphocyte reconstitution (Krautler et al., 2012). Thus, human LTBR knockin mice were generated to replace mouse Ltbr in NSGF mice.

In some embodiments, the NSGFL mice described herein comprise an inactivated mouse Flt3 allele and a nucleic acid encoding LTBR. In some embodiments, the nucleic acid encodes human LTBR. In some embodiments, the nucleic acid comprises a human LTBR transgene. In some embodiments, a transgene, such as a human LTBR transgene, is integrated into a mouse genome. In some embodiments, a human LTBR transgene comprises the nucleic acid sequence of SEQ ID NO: 4.

Human LTBR knockin mice, in some embodiments, are generated using a CRISPR/cas system. Cas9 mRNA, sgRNAs targeting mouse Ltbr and synthetic human LTBR minigene (encodes NM_002342 with all Exon and intron 1 sequences followed by a bGHpA STOP cassette) flanked by 5′ and 3′ mouse Ltbr homology sequence, for example, may be coinjected into fertilized NSGF oocytes (e.g., NOD.Cg-Prkdc^{scid} Il2rg^{tm1Wjl} Flt3^{em1Akp}). Human LTBR, in some embodiments, is inserted into exon 1 and exon 2 via homologous recombination. In some embodiments, the resulting founders, carrying human LTBR are bred, for example, to NOD.Cg-Prkdc^{scid} Il2rg^{tm1Wjl} Flt3^{em1Akp} mice, and are then interbred until all offspring were homozygous for the LTBR targeted mutation. Examples of primers that may be used for genotype by PCR reaction were listed in Table 2.

### The SGM3F Mouse Models

Further still, the present disclosure provides mouse models having an NSG™ background and further comprising an inactivated mouse Flt3 allele and nucleic acids encoding human IL3, GM-CSF, and SCF (referred to herein as SGM3F mice). In some embodiments, the genotype of an SGM3F mouse model is NSG™ Flt3^{em1Akp}-Tg(Hu-CMV-IL3, CSF2, KITLG)^{1Eav/MloySzJ} (see Example 5 for an exemplary method of generating the NSG™ Flt3^{em1Akp}-Tg(Hu-CMV-IL3, CSF2, KITLG)^{1Eav/MloySzJ} Mouse).

A limited biologic cross-reactivity between murine and human cytokines and cytokine receptors constrains the development of the human innate immune system, especially monocyte, macrophages and neutrophils. Efforts have been made to express human cytokines either through transgenic or knock-in human genes (Rathinam et al., 2011; Rongvaux et al., 2014; Willinger et al., 2011). One such variant of immunodeficient mice is based on NSG mice with transgenic expression of human Stem Cell Factor (SCF), Granulocyte Macrophage-Colony Stimulating Factor (GM-CSF) and Interleukin (IL)-3 (NSG-SGM3, SGM3) (Nicolini et al., 2004; Wunderlich et al., 2010). IL3 (e.g., NC_000005.10; chromosome:GRCh38:5:132060655-132063204), GM-CSF (e.g., NC_000005.10; chromosome:GRCh38:5:132073789-132076170) and SCF (e.g., NC_000012.12; chromosome: GRCh38:12:88492793-88580851) are cytokines and growth factors that promote the proliferation of a broad range of hematopoietic cell types. Initial studies demonstrated that, when transplanted with hCD34⁺ HPCs, SGM3 mice efficiently support the development of human immune cells, especially the CD33⁺ myeloid cells as well as CD4⁺Foxp3⁺ regulatory T cells, as compared to non-transgenic counterparts (Billerbeck et al., 2011). To further boost myeloid development, Flt3 mutant mice (NSGF) and SGM3 mice were crossed to yield SGM3F mice.

Thus, the SGM3F mice described herein comprise an inactivated mouse Flt3 allele and a nucleic acid encoding IL3, a nucleic acid encoding GM-CSF, and a nucleic acid encoding SCF. In some embodiments, the SGM3F mice comprise a nucleic acid encoding human IL3, a nucleic acid encoding human GM-CSF, and a nucleic acid encoding human SCF. In some embodiments, the SGM3F mice comprise a human IL3 transgene, a human CSF2 transgene, and a human KITLG transgene. In some embodiments, a transgene, such as a human IL3, CSF2, and/or KITLG transgene, is integrated into a mouse genome. Human IL3, CSF2, and KITLG transgenes are described (Nicolini et al., 2004), incorporated by reference herein.

SGM3F mice, in some embodiments, are generated by crossing NSG-SGM3 mice (NOD.Cg-Prkdc^{scid}Il2rg^{tm1Wjl} Tg(CMV-IL3,CSF2,KITLG)^{1Eav/MloySzJ}; RRID:IMSR JAX:013062) to NSGF mice and interbreeding until all offspring are homozygous. NSG-SGM3 mice carry three separate transgenes which were designed each carrying one of the human interleukin-3 (IL-3) gene, the human granulocyte/macrophage-stimulating factor (GM-CSF) gene, or human Steel factor (SF) gene. Expression of each gene is driven by a human cytomegalovirus promoter/enhancer sequence and is followed by a human growth hormone cassette and a polyadenylation (polyA) sequence (Nicolini et al., 2004). The transgenes were microinjected into fertilized C57BL/6×C3H/HeN oocytes. The resulting founders, carrying all three transgenes (3GS), in some embodiments, are backcrossed to BALB/c-scid/scid mice for several generations and subsequently backcrossed to NOD.CB17-Prkdc^{scid} mice for multiple (e.g., at least 11) generations. These mice may then be bred to NSG mice (NOD.Cg-Prkdc^{scid} Il2rg^{tm1Wjl}; RRID:IMSR JAX: 005557), for example, and then interbred until all offspring are homozygous for 3GS and the IL2rg targeted mutation. The transgenic mice may be bred to NSG mice for at least one generation to establish NSG-SGM3 mice. NSGF mice may be generated, for example, using the CRISPR/cas system. Cas9 mRNA and sgRNAs targeting mouse Flt3, in some embodiments, are coinjected into fertilized NSG oocytes. The resulting founders, carrying Flt3 deletion may be bred to NSG mice, and then interbred until all offspring are homozygous for Flt3 targeted mutation.

### Human Immune System Model

The mouse models of the present disclosure (e.g., the NSGF, NSGF6, NSGFT, NSGFL, or SGM3F mouse model, or any combination thereof), in some embodiments, are used to support human CD34⁺ HSCs and development of a human innate immune system. The human immune system includes the innate immune system and the adaptive immune system. The innate immune system is responsible for recruiting immune cells to sites of infection, activation of the complement cascade, the identification and removal of foreign substances from the body by leukocytes, activation of the adaptive immune system, and acting as a physical and chemical barrier to infectious agents.

In some embodiments, a mouse model provided herein (e.g., the NSGF, NSGF6, NSGFT, NSGFL, or SGM3F mouse model, or any combination thereof) is sublethally irradiated (e.g., 100-300 cGy) to kill resident mouse HSCs, and then the irradiated mouse is engrafted with human CD34⁺ HSCs (e.g., 50,000 to 200,000 HSCs) to initiate the development of a human innate immune system. Thus, in some embodiments, a mouse further comprises human CD34⁺ HSCs. Human CD34⁺ HSCs may be from any source including, but not limited to, human fetal liver, human umbilical cord blood, mobilized peripheral blood, and bone marrow. In some embodiments, human CD34⁺ HSCs are from human umbilical cord blood.

The differentiation of human CD34⁺ HSCs into divergent immune cells (e.g., T cells, B cells, dendritic cells) is a complex process in which successive developmental steps are regulated by multiple cytokines. This process can be monitored through cell surface antigens, such as cluster of differentiation (CD) antigens. CD45, for example, is expressed on the surface of HSCs, macrophages, monocytes, T cells, B cells, natural killer cells, and dendritic cells, thus can be used as a marker indicative of engraftment. On T cells, CD45 regulates T cell receptor signaling, cell growth, and cell differentiation. In some embodiments, a mouse model (e.g., the NSGF, NSGF6, NSGFT, NSGFL, or SGM3F mouse model, or any combination thereof) comprises human CD45⁺ cells. In some embodiments, a mouse model (e.g., the NSGF, NSGF6, NSGFT, NSGFL, or SGM3F mouse model, or any combination thereof) also exhibits engraftment of human CD45⁺ cells to tissues, but not limited to, in the lung, thymus, spleen, lymph nodes, and/or small intestine.

As CD45⁺ cells mature, they begin to express additional biomarkers, indicative of the various developmental stages and differentiating cell types. Developing T cells, for example, also express CD3, CD4, and CD8. As another example, developing myeloid cells express CD33⁺. A mouse model (e.g., the NSGF, NSGF6, NSGFT, NSGFL, or SGM3F mouse model, or any combination thereof) herein, in some embodiments, comprises not only human CD45⁺ cells but also double positive human CD45⁺/CD3⁺ T cells as well as double positive human CD45⁺/CD33⁺ myeloid cells.

Thus, in some embodiments, a population of human CD45⁺ cells in a mouse model (e.g., the NSGF, NSGF6, NSGFT, NSGFL, or SGM3F mouse model, or any combination thereof) comprises human CD45⁺/CD3⁺ T cells. In some embodiments, the population of human CD45⁺ cells comprises an increased percentage of human CD45⁺/CD3⁺ T cells, relative to an NSG™ control mouse. In some embodiments, the percentage of human CD45⁺/CD3⁺ T cells in a mouse model (e.g., the NSGF, NSGF6, NSGFT, NSGFL, or SGM3F mouse model, or any combination thereof) is increased by at least 25%, relative to an NSG™ control mouse. For example, the percentage of human CD45⁺/CD3⁺ T cells in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD3⁺ T cells in a mouse model is increased by at least 50%, relative to an NSG™ control mouse. In some embodiments, the percentage of human CD45⁺/CD3⁺ T cells in a mouse model is increased by at least 100%, relative to an NSG™ control mouse. In some embodiments, the percentage of human CD45⁺/CD3⁺ T cells in a mouse model is increased by 25%-100%, 25%-75%, 25%-50%, 50%-100%, 50%-75%, or 75%-100%, relative to an NSG™ control mouse.

In some embodiments, a population of human CD45⁺ cells in a mouse model (e.g., the NSGF, NSGF6, NSGFT, NSGFL, or SGM3F mouse model, or any combination thereof) comprises human CD45⁺/CD33⁺ myeloid cells. In some embodiments, the population of human CD45⁺ cells comprise an increased percentage of human CD45⁺/CD33+ myeloid cells, relative to an NSG™ control mouse. In some embodiments, the percentage of human CD45⁺/CD33⁺ myeloid cells in a mouse model is increased by at least 25%, relative to an NSG™ control mouse. For example, the percentage of human CD45⁺/CD33⁺ myeloid cells in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD33⁺ myeloid cells in a mouse model is increased by at least 50%, relative to an NSG™ control mouse. In some embodiments, the percentage of human CD45⁺/CD33⁺ myeloid cells in a mouse model is increased by at least 100%, relative to an NSG™ control mouse. In some embodiments, the percentage of human CD45⁺/CD33⁺ myeloid cells in a mouse model is increased by 25%-100%, 25%-75%, 25%-50%, 50%-100%, 50%-75%, or 75%-100%, relative to an NSG™ control mouse.

In some embodiments, a population of human CD45+ cells in a mouse model (e.g., the NSGF, NSGF6, NSGFT, NSGFL, or SGM3F mouse model, or any combination thereof) comprises human CD45⁺/CD19⁺ B cells. In some embodiments, the population of human CD45+ cells comprises an decreased percentage of human CD45⁺/CD19⁺ B cells, relative to an NSG™ control mouse. In some embodiments, the percentage of human CD45⁺/CD19⁺ B cells in a mouse model is decreased by at least 25%, relative to an NSG™ control mouse. For example, the percentage of human CD45⁺/CD19⁺ B cells in a mouse model may be decreased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD45⁺/CD19⁺ B cells in a mouse model is decreased by at least 50%, relative to an NSG™ control mouse. In some embodiments, the percentage of human CD45⁺/CD19⁺ B cells in a mouse model is decreased by at least 100%, relative to an NSG™ control mouse. In some embodiments, the percentage of human CD45⁺/CD19⁺ B cells in a mouse model is decreased by 25%-100%, 25%-75%, 25%-50%, 50%-100%, 50%-75%, or 75%-100%, relative to an NSG™ control mouse.

The mouse models provided herein (e.g., the NSGF, NSGF6, NSGFT, NSGFL, or SGM3F mouse model, or any combination thereof), surprisingly, are also capable of supporting engraftment of dendritic cells (e.g., plasmacytoid dendritic cells and myeloid dendritic cells), natural killer cells, and monocyte-derived macrophages (monocyte macrophages). Plasmacytoid dendritic cells (pDCs) secrete high levels of interferon alpha; myeloid dendritic cells (mDCs) secrete interleukin 12, interleukin 6, tumor necrosis factor, and chemokines; natural killer cells destroy damaged host cells, such as tumor cells and virus-infected cells; and macrophages consume substantial numbers of bacteria or other cells or microbes.

In some embodiments, a mouse model provided herein (e.g., the NSGF, NSGF6, NSGFT, NSGFL, or SGM3F mouse model, or any combination thereof) comprises an increased percentage of human CD14⁺ monocytes or macrophages, relative to an NSG™ control mouse. In some embodiments, the percentage of human CD14⁺ monocytes or macrophages in a mouse model is increased by at least 25%, relative to an NSG™ control mouse. For example, the percentage of human CD14⁺ monocytes or macrophages in a mouse model may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD14⁺ monocytes or macrophages in a mouse model is increased by at least 50%, relative to an NSG™ control mouse. In some embodiments, the percentage of human CD14+ monocytes or macrophages in a mouse model is increased by at least 100%, relative to an NSG™ control mouse. In some embodiments, the percentage of human CD14⁺ monocytes or macrophages in a mouse model is increased by 25%-100%, 25%-75%, 25%-50%, 50%-100%, 50%-75%, or 75%-100%, relative to an NSG™ control mouse.

In some embodiments, an SGM3F mouse comprises an increased percentage of human CD66b+ cells, relative to an NSG™ control mouse and/or an NSGF control mouse. In some embodiments, the percentage of human CD66b⁺ cells in the SGM3F mouse is increased by at least 25%, relative to an NSG™ control mouse and/or an NSGF control mouse. For example, the percentage of human CD66b⁺ cells in the NSG™ SGM3F mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse and/or an NSGF control mouse. In some embodiments, the percentage of human CD66b⁺ cells in the SGM3F mouse is increased by at least 50%, relative to an NSG™ control mouse and/or an NSGF control mouse. In some embodiments, the percentage of human CD11C⁺ HLA-DR⁺ myeloid dendritic cells in the SGM3F mouse is increased by at least 100%, relative to an NSG™ control mouse and/or an NSGF control mouse. In some embodiments, the percentage of human CD66b⁺ cells in the SGM3F mouse is increased by 25%-100%, 25%-75%, 25%-50%, 50%-100%, 50%-75%, or 75%-100%, relative to an NSG™ control mouse and/or an NSGF control mouse. In some embodiments, an SGM3F mouse comprises an increased percentage of human CD11c⁺ myeloid dendritic cells, relative to an NSG™ control mouse and/or an NSGF control mouse. In some embodiments, the percentage of human CD11c⁺ HLA-DR⁺ myeloid dendritic cells in the SGM3F mouse is increased by at least 25%, relative to an NSG™ control mouse and/or an NSGF control mouse. For example, the percentage of human CD11c⁺ HLA-DR⁺ myeloid dendritic cells in the NSG™ SGM3F mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse and/or an NSGF control mouse. In some embodiments, the percentage of human CD11c HLA-DR⁺ myeloid dendritic cells in the SGM3F mouse is increased by at least 50%, relative to an NSG™ control mouse and/or an NSGF control mouse. In some embodiments, the percentage of human CD11c⁺ HLA-DR⁺ myeloid dendritic cells in the SGM3F mouse is increased by at least 100%, relative to an NSG™ control mouse and/or an NSGF control mouse. In some embodiments, the percentage of human CD11c⁺ HLA-DR⁺ myeloid dendritic cells in the SGM3F mouse is increased by 25%-100%, 25%-75%, 25%-50%, 50%-100%, 50%-75%, or 75%-100%, relative to an NSG™ control mouse and/or an NSGF control mouse.

In some embodiments, an NSGF mouse comprises an increased percentage of human CD303⁺ plasmacytoid dendritic cells, relative to an NSG™ control mouse. In some embodiments, the percentage of human CD303+ plasmacytoid dendritic cells in the NSGF mouse is increased by at least 25%, relative to an NSG™ control mouse. For example, the percentage of human CD303⁺ plasmacytoid dendritic cells in the NSGF mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse. In some embodiments, the percentage of human CD303⁺ plasmacytoid dendritic cells in the NSGF mouse is increased by at least 50%, relative to an NSG™ control mouse. In some embodiments, the percentage of human CD303⁺ plasmacytoid dendritic cells in the NSGF mouse is increased by at least 100%, relative to an NSG™ control mouse. In some embodiments, the percentage of human CD303⁺ plasmacytoid dendritic cells in the NSGF mouse is increased by 25%-100%, 25%-75%, 25%-50%, 50%-100%, 50%-75%, or 75%-100%, relative to an NSG™ control mouse.

In some embodiments, an SGM3F mouse comprises an increased percentage of human proportion of CCR7⁻ effector T cells, relative to an NSG™ control mouse and/or an NSGF control mouse. In some embodiments, the percentage of human proportion of CCR7⁻ effector T cells in the SGM3F mouse is increased by at least 25%, relative to an NSG™ control mouse and/or an NSGF control mouse. For example, the percentage of human proportion of CCR7⁻ effector T cells in the NSG™ SGM3F mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse and/or an NSGF control mouse. In some embodiments, the percentage of human proportion of CCR7⁻ effector T cells in the SGM3F mouse is increased by at least 50%, relative to an NSG™ control mouse and/or an NSGF control mouse. In some embodiments, the percentage of human proportion of CCR7⁻ effector T cells in the SGM3F mouse is increased by at least 100%, relative to an NSG™ control mouse. In some embodiments, the percentage of human proportion of CCR7⁻ effector T cells in the SGM3F mouse is increased by 25%-100%, 25%-75%, 25%-50%, 50%-100%, 50%-75%, or 75%-100%, relative to an NSG™ control mouse and/or an NSGF control mouse.

In some embodiments, an SGM3F mouse comprises an increased percentage of total human IgG, relative to an NSG™ control mouse and/or an NSGF control mouse. In some embodiments, the percentage of total human IgG in the SGM3F mouse is increased by at least 25%, relative to an NSG™ control mouse and/or an NSGF control mouse. For example, the percentage of total human IgG in the NSG™ SGM3F mouse may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to an NSG™ control mouse and/or an NSGF control mouse. In some embodiments, the percentage of total human IgG in the SGM3F mouse is increased by at least 50%, relative to an NSG™ control mouse and/or an NSGF control mouse. In some embodiments, the percentage of total human IgG in the SGM3F mouse is increased by at least 100%, relative to an NSG™ control mouse and/or an NSGF control mouse. In some embodiments, the percentage of total human IgG in the SGM3F mouse is increased by 25%-100%, 25%-75%, 25%-50%, 50%-100%, 50%-75%, or 75%-100%, relative to an NSG™ control mouse and/or an NSGF control mouse.

In some embodiments, an SGM3F mouse comprises a significant functional improvement of the human immune system relative to a SGM3 control mouse. For example, an SGM3F mouse may comprise increased specific TgG to KLH following vaccination with alum-adjuvanted Tdap/KLH vaccine IP/SC relative to a SGM3 control mouse. In some embodiments, an SGM3F mouse comprises increased specific IgG to Fluzone following vaccination with Fluzone IV/IP relative to a SGM3 control mouse. In some embodiments, an SGM3F mouse comprises neutralizing antibody to H1N1 FluA/Cal9 virus, but not to influenza B virus as measured by hemagglutination inhibition assay relative to a SGM3 control mouse.

In some embodiments, an NSGF mouse of the present disclosure is used to support human hematopoietic cell engraftment and human myelopoiesis.

In some embodiments, an NSGF6 mouse of the present disclosure is used to support human hematopoietic cell engraftment, human myelopoiesis, and human lymphopoiesis.

In some embodiments, an NSGFT mouse of the present disclosure is used to support human hematopoietic cell engraftment, human myelopoiesis, and human lymphopoiesis.

In some embodiments, an NSGFL mouse of the present disclosure, in some embodiments, is used to support the development of human lymphoid tissue, particularly the adaptive immune response and germinal center formation.

The SGM3F mouse of the present disclosure, in some embodiments, is used to support engraftment of myeloid lineages and regulatory T cell populations.

### Methods of Producing Transgenic Animals

Provided herein, in some aspects, are methods of producing a transgenic animal that expresses human IL6, human TSLP, human LTBR, human IL3, human GM-CSF, human SCF, or any combination thereof. A transgenic animal, herein, refers to an animal that has a foreign (exogenous) nucleic acid (e.g., transgene) inserted into (integrated into) its genome. In some embodiments, the transgenic animal is a transgenic rodent, such as a mouse or a rat. In some embodiments, the transgenic animal is a mouse. Three conventional methods used for the production of transgenic animals include DNA microinjection (Gordon & Ruddle, 1981), incorporated herein by reference), embryonic stem cell-mediated gene transfer (Gossler, Doetschman, Korn, Serfling, & Kemler, 1986), incorporated herein by reference) and retrovirus-mediated gene transfer (Jaenisch, 1976), incorporated herein by reference), any of which may be used as provided herein. Electroporation may also be used to produce transgenic mice (see, e.g., WO 2016/054032 and WO 2017/124086, each of which is incorporated herein by reference).

A nucleic acid encoding human IL6, human TSLP, human LTBR, human IL3, human GM-CSF, human SCF, or any combination thereof, in some embodiments, comprises a transgene, for example, a transgene that comprises a promoter (e.g., a constitutively active promoter) operably linked to a nucleotide sequence encoding human IL6, human TSLP, human LTBR, human IL3, human GM-CSF, human SCF, or any combination thereof. In some embodiments, a nucleic acid encoding human IL6, human TSLP, human LTBR, human IL3, human GM-CSF, human SCF, or any combination thereof used to produce a transgenic animal (e.g., mouse) is present on an vector, such as a plasmid, a bacterial artificial chromosome (BAC), or a yeast artificial chromosome (YAC), which is delivered, for example, to the pronucleus/nucleus of a fertilized embryo where the nucleic acid randomly integrates into the animal genome. In some embodiments, the fertilized embryo is a single-cell embryo (e.g., a zygote). In some embodiments, the fertilized embryo is a multi-cell embryo (e.g., a developmental stage following a zygote, such as a blastocyst). In some embodiments, the nucleic acid (e.g., carried on a BAC) is delivered to a fertilized embryo of an NSG™ mouse to produce a mouse model of the present disclosure (e.g., the NSGF, NSGF6, NSGFT, NSGFL, or SGM3F mouse model, or any combination thereof). Following injection of the fertilized embryo, the fertilized embryo may be transferred to a pseudopregnant female, which subsequently gives birth to offspring comprising the nucleic acid encoding human IL6, human TSLP, human LTBR, human IL3, human GM-CSF, human SCF, or any combination thereof. The presence or absence of the nucleic acid encoding human IL6, human TSLP, human LTBR, human IL3, human GM-CSF, human SCF, or any combination thereof may be confirmed, for example, using any number of genotyping methods (e.g., sequencing and/or genomic PCR).

In some embodiments, a CRISPR system is used to generate deletion in specific target sites encoding endogenous mouse 116, mouse Tslp, or mouse Ltbr of a mouse model provided herein (e.g., the NSGF, NSGF6, NSGFT, NSGFL, mouse model, or any combination thereof). By coinjecting donor DNA encoding human IL6, human TSLP, or human LTBR, gene editing is achieved precisely by homology-directed repair (See, e.g. (Yang et al., 2013), which is incorporated by reference herein). For example, Cas9 mRNA or protein, one or multiple guide RNAs (gRNAs) and donor plasmid template encompassing the human IL6 gene flanked by 5′ and 3′ mouse 116 homology sequence can be injected directly into mouse embryos to generate precise genomic edits into a 116 gene. Mice that develop from these embryos can be genotyped or sequenced to determine if they carry the desired transgene, and those that do may be bred to confirm germline transmission.

Also provided herein are methods of inactivating an endogenous Flt3 allele. In some embodiments, an endogenous Flt3 allele is inactivated in a transgenic animal. In some embodiments, a gene/genome editing method is used for gene (allele) inactivation. Engineered nuclease-based gene editing systems that may be used as provided herein include, for example, clustered regularly interspaced short palindromic repeat (CRISPR) systems, zinc-finger nucleases (ZFNs), and transcription activator-like effector nucleases (TALENs). See, e.g., (Carroll, 2011; Gaj, Gersbach, & Barbas, 2013; Joung & Sander, 2013), each of which is incorporated by reference herein.

In some embodiments, a CRISPR system is used to inactivate an endogenous Flt3 allele of a mouse model provided herein (e.g., the NSGF, NSGF6, NSGFT, NSGFL, or SGM3F mouse model, or any combination thereof). See, e.g., (Harms et al., 2014; Inui et al., 2014), each of which are incorporated by reference herein). For example, Cas9 mRNA or protein and one or multiple guide RNAs (gRNAs) can be injected directly into mouse embryos to generate precise genomic edits into a Flt3 gene. Mice that develop from these embryos can be genotyped or sequenced to determine if they carry the desired mutation(s), and those that do may be bred to confirm germline transmission.

The CRISPR/Cas system is a naturally occurring defense mechanism in prokaryotes that has been repurposed as a RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target to be modified. Thus, one can change the genomic target of the Cas protein by simply changing the target sequence present in the gRNA. In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence (e.g., a region of the Flt3 allele). See, e.g., (Deltcheva et al., 2011; 25 Jinek et al., 2012), each of which is incorporated by reference herein. In some embodiments, a gRNA used in the methods provided herein binds to a region (e.g., exon 3) of a mouse Flt3 allele. In some embodiments, the gRNA that binds to a region of a mouse Flt3 allele comprises the nucleotide sequence of 5′-AAGTGCAGCTCGCCACCCCA-3′ (SEQ ID NO: 5). In some embodiments, gRNAs used in the methods provided herein binds to regions (e.g., exon 1 and exon 5) of a mouse Il6 allele. In some embodiments, the gRNAs that binds to regions of a mouse 116 allele comprises the nucleotide sequences of 5′-AGGAACTTCATAGCGGTTTC-3′ (SEQ ID NO: 6) and 5′-ATGCTTAGGCATAACGCACT-3′ (SEQ ID NO: 7). In some embodiments, gRNAs used in the methods provided herein binds to regions (e.g., exon 1 and exon 5) of a mouse Tslp allele. In some embodiments, the gRNAs that binds to regions of a mouse Tslp allele comprises the nucleotide sequences of 5′-CCACGTTCAGGCGACAGCAT-3′ (SEQ ID NO: 8) and 5′-TTATTCTGGAGATTGCATGA-3′ (SEQ ID NO: 9). In some embodiments, gRNAs used in the methods provided herein binds to regions (e.g., exon 1 and exon 2) of a mouse Ltbr allele. In some embodiments, the gRNAs that binds to regions of a mouse Ltbr allele comprises the nucleotide sequences of 5′-GCTCGGCTGACCAGACCGGG-3′(SEQ ID NO: 10) and 5′-GAGCCACTGTTCTCACCTGG-3′ (SEQ ID NO: 11).

### Methods of Use

The mouse models provided herein (e.g., the NSGF, NSGF6, NSGFT, NSGFL, or SGM3F mouse model, or any combination thereof) may be used for any number of applications. For example, a mouse model may be used to test how a particular agent (e.g., therapeutic agent) or medical procedure (e.g., tissue transplantation) impacts the human innate immune system (e.g., human innate immune cell responses) and human adaptive immune system (e.g., antibody response).

In some embodiments, a mouse model (e.g., the NSGF, NSGF6, NSGFT, NSGFL, or SGM3F mouse model, or any combination thereof) is used to evaluate an effect of an agent on human innate immune system development. Thus, provided herein are methods that comprise administering an agent to a mouse model, and evaluating an effect of the agent on human innate immune system development in the mouse. Effects of an agent may be evaluated, for example, by measuring a human innate immune cell (e.g., T cell and/or dendritic cell) response (e.g., cell death, cell signaling, cell proliferation, etc.) and human adaptive immune response (e.g., antibody production). Non-limiting examples of agents include therapeutic agents, such as anti-cancer agents and anti-inflammatory agents, and prophylactic agents, such as immunogenic compositions (e.g., vaccines).

In other embodiments, a mouse model (e.g., the NSGF, NSGF6, NSGFT, NSGFL, or SGM3F mouse model, or any combination thereof) is used to evaluate an immunotherapeutic response to a human tumor. Thus, provided herein are methods that comprise administering an agent to a mouse model that has a human tumor, and evaluating an effect of the agent on the human innate immune system and/or on the tumor in the mouse. Effects of an agent may be evaluated by measuring a human innate immune cell (e.g., T cell and/or dendritic cell) response, human adaptive immune response (e.g., antibody production) and/or tumor cell response (e.g., cell death, cell signaling, cell proliferation, etc.). In some embodiments, the agent is an anti-cancer agent.

In yet other embodiments, a mouse model (e.g., the NSGF, NSGF6, NSGFT, NSGFL, or SGM3F mouse model, or any combination thereof) is used to evaluate a human immune response to an infectious microorganism. Thus, provided herein are methods that comprise exposing a mouse model to an infectious microorganism (e.g., bacteria and/or virus), and evaluating an effect of the infectious microorganism on the human immune response. Effects of an infectious microorganism may be evaluated by measuring a human innate immune cell (e.g., T cell and/or dendritic cell) response (e.g., cell death, cell signaling, cell proliferation, etc.) and human adaptive immune response (e.g., antibody production). These methods may further comprise administering a drug or an anti-microbial agent (e.g., an anti-bacterial agent or an anti-viral agent) to the mouse and evaluating an effect of the drug or anti-microbial agent on the infectious microorganism.

In still further embodiments, a mouse model (e.g., the NSGF, NSGF6, NSGFT, NSGFL, or SGM3F mouse model, or any combination thereof) is used to evaluate a human immune response to tissue transplantation. Thus, provided herein are methods that comprise transplanting tissue (e.g., allogeneic tissue) to a mouse model and evaluating an effect of the transplanted tissue on the human innate immune response. Effects of a transplanted tissue may be evaluated by measuring a human innate immune cell (e.g., T cell and/or dendritic cell) response (e.g., cell death, cell signaling, cell proliferation, etc.) and human adaptive immune response (e.g., antibody production) to the transplanted tissue.

### EXAMPLES

Mouse Flt3 KO creates space for human DCs and, by making the receptor ligand Flt3L available to human cells, improves the development of human myeloid cells upon transplant with human CD34⁺ HPCs. Side-by-side comparison of the SGM3 mice and the NSGF mice generated herein revealed some similarities but also substantial differences between the two strains, for example: (1) NSGF mice support human hematopoiesis upon transplant of cord blood as well as adult bone marrow HPCs; (2) NSGF mice support differentiation of human DC subsets; and (3) hSGM3 mice can generate human antibody titers. These results motivated us to cross the two strains to generate a novel strain, SGM3F. hSGM3F mice therefore represent a step towards an improved model because our studies show that these mice support the generation of antibody responses upon vaccination—an outcome that can be attributed to human myeloid cells. In line with this, we generated multiple improved immunodeficient mice using CRISPR technology. By crossing each strain of mice, we aim to combine various the features of the human transgenes to obtain mouse models with the capacity to develop various subsets of human immune cells and to mount specific immune response upon reconstitution with human HPCs.

### Example 1. The NOD.Cg-Prkdc^{scid} Il2rg^{tm1Wjl}-Flt3^{em1Akp} (NSGF) Mouse Model

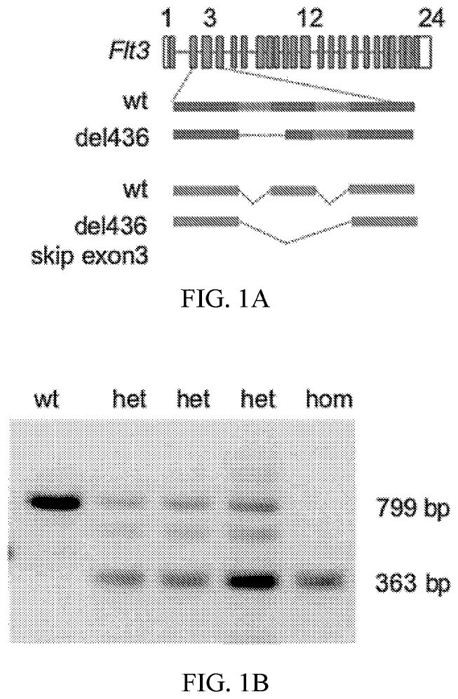

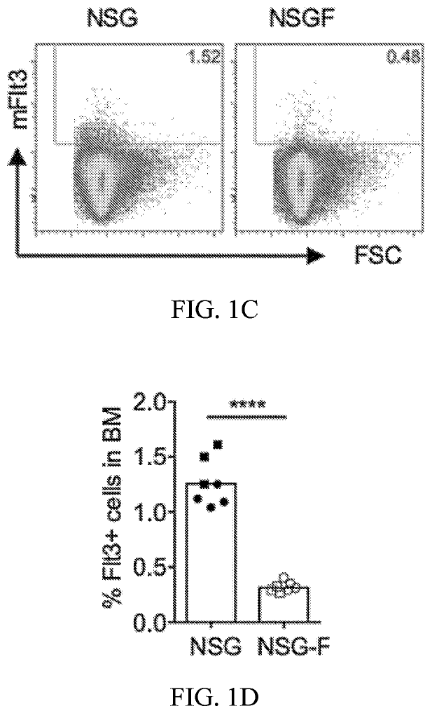

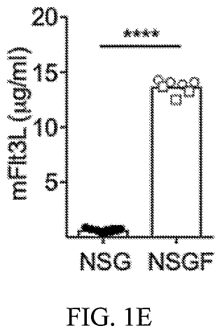

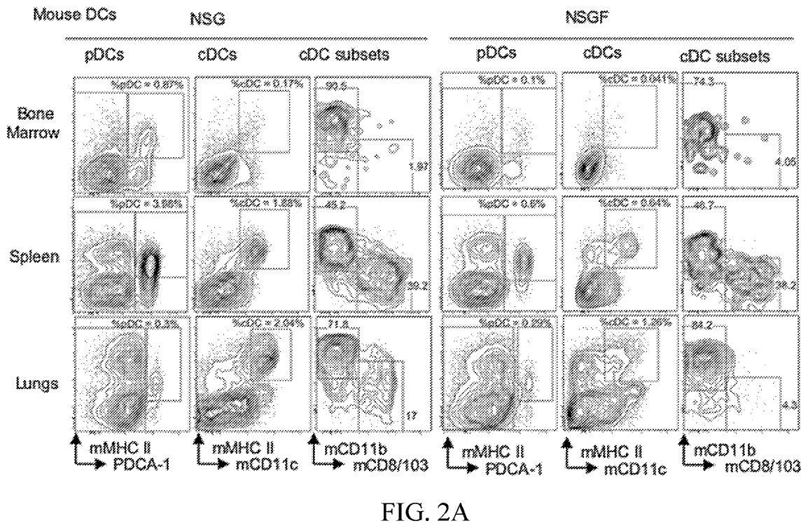

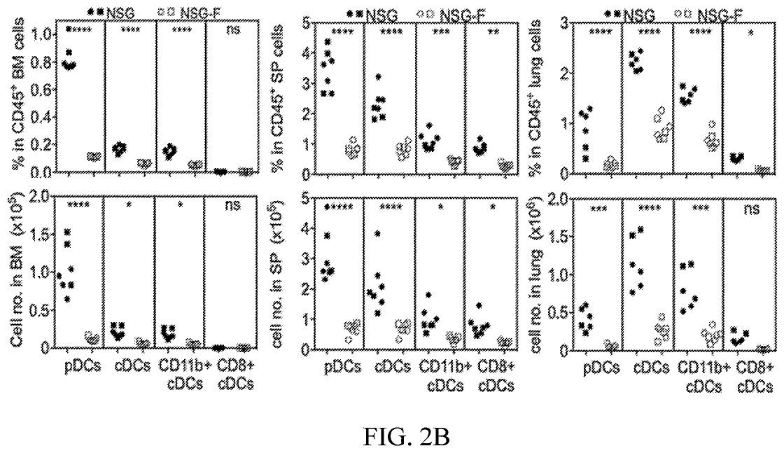

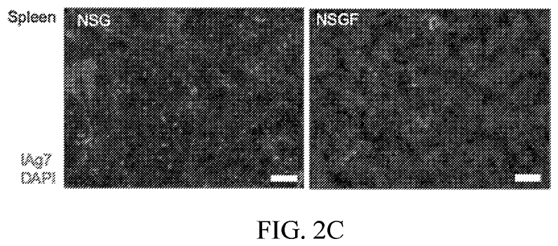

Flt3 is a receptor important for development of the dendritic cells and monocytic lineages. Flt3L-Flt3 signaling is important for the development of various DC and monocytic lineages (Ding et al., 2014; Ginhoux et al., 2009; McKenna et al., 2000; Waskow et al., 2008) and it's role is further supported by the increase of circulating conventional (c)DCs and plasmacytoid (p)DCs after the administration of Flt3L in vivo in mice and humans (Karsunky et al., 2003; Maraskovsky et al., 1996; Pulendran et al., 2000). Knocking-out mouse Flt3 can lead to: 1. decrease in murine DCs and other myeloid cells; and 2. increase in the availability of mouse Flt3L (which can act via human receptor) to human cells, thereby improving the long-term development of human myeloid cells upon transplant with human CD34⁺ HPCs. Thus, we used CRISPR/Cas system to generate a Flt3 KO mouse in NSG background. Founder mice carrying a chromosomal deletion at the exon 3 were backcross to NSG and inbred to obtain homozygous Flt3^{-/-} allele (FIG. 1A) and yield NOD.Cg-Prkdc^{scid} Il2rg^{tm1Wjl}-Flt3^{em1Akp} mice (NSGF). The Flt3 genotype was confirmed by PCR with a 363 bp product and Sanger sequence (FIG. 1B). Consistently, we observed a decrease of mouse Flt3 expression in bone marrow cells (FIG. 1C-1D) and an increase amount of mouse Flt3L in the plasma (FIG. 1E). To check the impact of Flt3 KO on mouse DC development, we analyzed different subsets of mouse DCs including PDCA-1⁺ pDCs, CD11c⁺ cDCs. Murine cDCs were further divided into CD11b⁺ or CD8⁺ subsets in the bone marrow and spleen and CD103⁺ subsets in the lungs. (FIG. 2A). To this end, we observed an 80-90% decrease in DC subsets in the bone marrow, spleen and lungs of NSGF mice in comparison to age and gender matched NSG mice by FACS (FIG. 2A-2B). This was further confirmed by the scarcity of mouse MHC class II (I-A^{g7})+ cells in the spleen by immunofluorescent staining (FIG. 2C). Overall, our data confirmed a functional deletion of mouse Flt3 in NSGF mice.

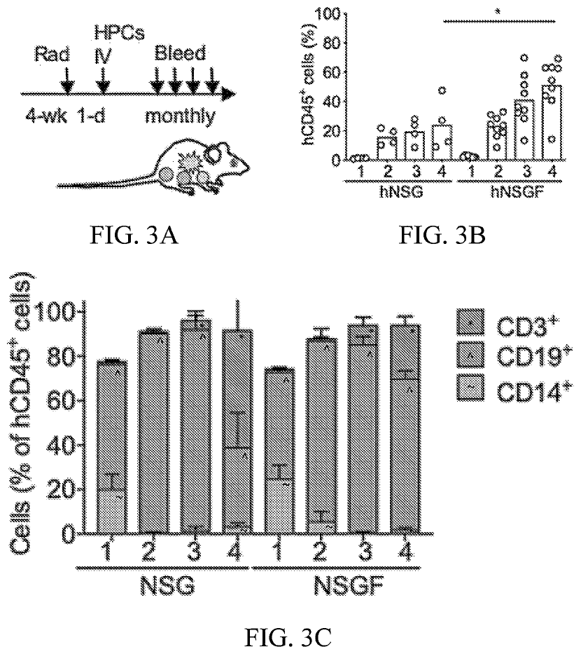

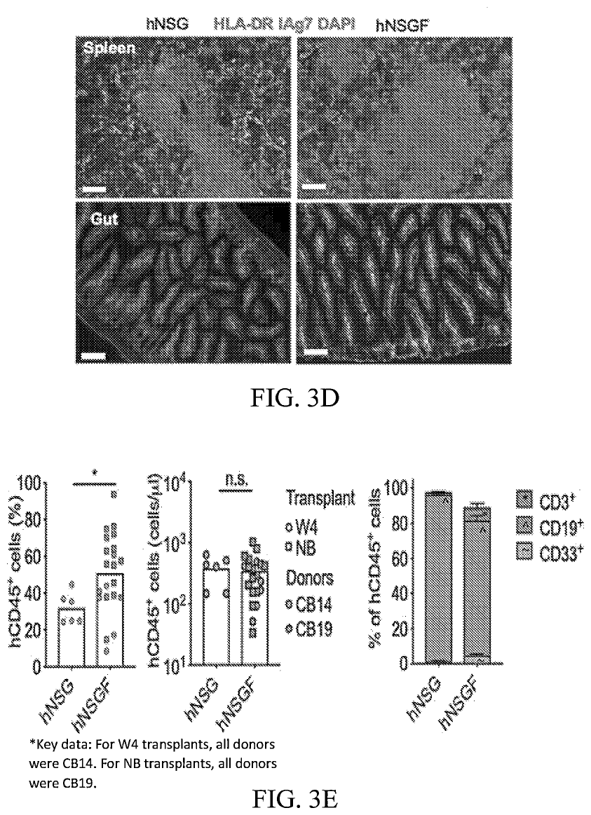

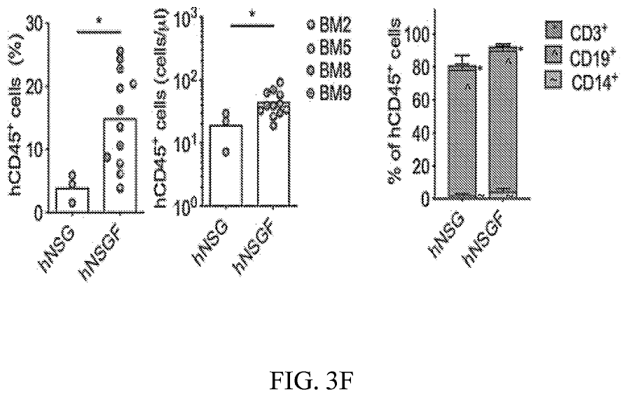

One question was whether deletion of mouse DCs will improve human engraftment and generate “space” for human DCs. To this end, sublethally irradiated NSGF mice were transplanted with 1×10⁵ fetal liver CD34⁺ HPCs, and the engraftment of human cells was measured in the blood at different time points after transplantation (FIG. 3A). As shown in FIG. 3B, humanized (h) NSGF mice allowed a higher reconstitution of human CD45⁺ immune cells in the blood with different lineages of human cells including CD14⁺ monocytes, CD19⁺ B cells, CD3⁺ T cells after transplantation by FACS (FIG. 3C). In addition, we also observed a lack of mouse MHC class II (I-A^{g}7)⁺ cells and the development of HLA-DR⁺ cells in the spleen and colonization of mucosal tissues with human DCs by the presence of HLA-DR⁺ cells in the lamina propria of the small intestine with the morphology of DCs (FIG. 3D). To test it's capacity to support the engraftment of non-fetal HPCs, we irradiated newborn or 4-weeks old mice sub-lethally and transplanted with 1×10⁵ CD34⁺ HPCs from cord blood or from adult bone marrow (FIG. 3E-3F). hNSGF mice demonstrate enhancement of hCD45⁺ engraftment at 12⁻ weeks post-transplant with slight expansion of both CD33⁺ myeloid cells and CD3⁺ T cells in the blood (FIG. 3E). In addition, hNSGF mice transplanted with adult bone marrow HPCs at the limiting number (1×10⁵) demonstrated a significant improvement in hCD45⁺ engraftment in the blood (FIG. 3F). The improvement was reflected in the percentage and absolute cell count of hCD45⁺ cells in the blood (FIG. 3F). Thus, mouse Flt3 knock-out led to a decrease in murine DCs and an increase in the availability of mouse Flt3 ligand to human cells, thereby improving the long-term development of human myeloid cells upon transplant with human CD34⁺ hematopoietic progenitor cells.

Generation of Mouse Model: Mouse Flt3 knockout mice (NOD.Cg-Prkdc^{scid} Il2rg^{tm1Wjl} Flt3^{em1Akp}) were generated by CRISPR using Cas9 mRNA and sgRNAs (5′-AAGTGCAGCTCGCCACCCCA-3′, SEQ ID NO: 5) targeting exon 3 of mouse Flt3 in fertilized eggs of NSG mice (NOD.Cg-Prkdc^{scid} Il2rg^{tm1Wjl}/SzJ; RRID:JMSR JAX:005557). The blastocysts derived from the injected embryos were transplanted into foster mothers and newborn pups were obtained. Mice carrying a null deletion were backcrossed to NSG. F0 and F1 littermates were tail tipping and tested for successful gene-knockout by PCR and Sanger sequencing. Forward primer (5′-GGTACCAGCAGAGTTGGATAGC-3′, SEQ ID NO: 12) and reverse primers (5′-ATCCCTTACACAGAAGCTGGAG-3′, SEQ ID NO: 13) were used in a PCR reaction to detect the mouse Flt3 wildtype (WT) allele from mutant allele (Table 2). The WT allele yields a DNA fragment 799 bp in length, whereas the mutated allele generates a DNA fragment of 363 bp in length.

### Example 2. The NOD.Cg-Prkdc^{scid}Il2rg^{tm1Wjl}-Flt3^{em1Akp}Il6^{em1(IL6)Akp} (NSGF6) Mouse Model

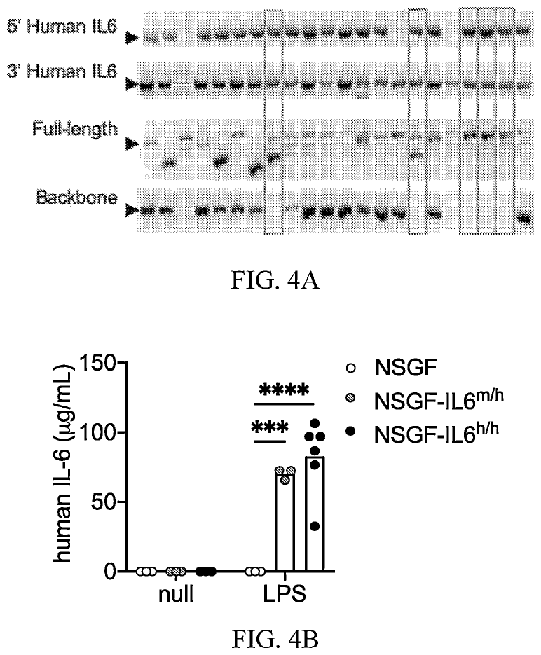

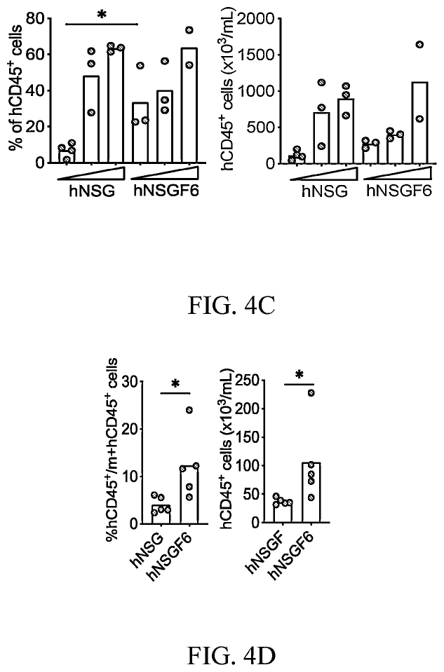

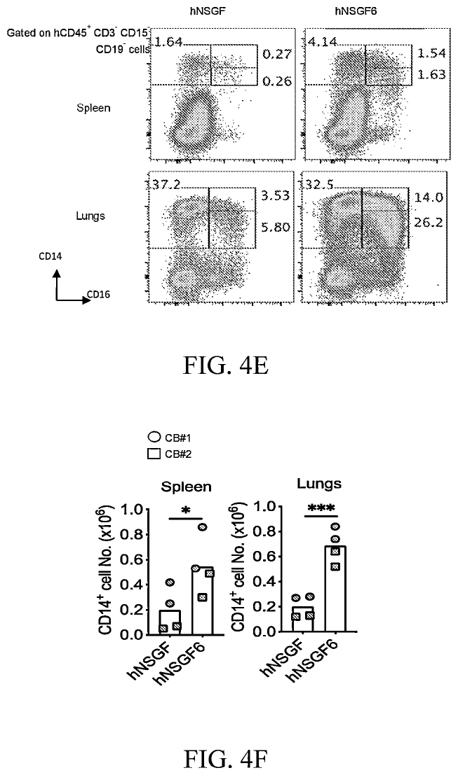

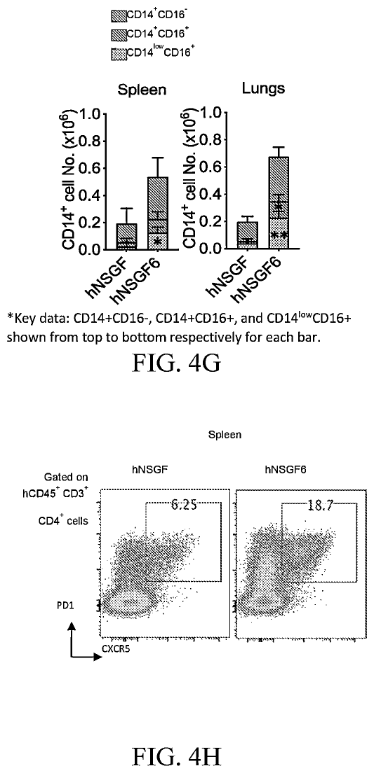

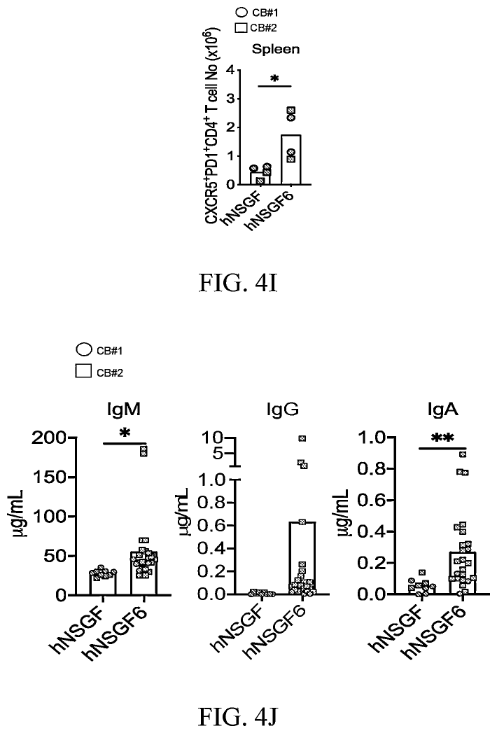

IL6 is essential in HPC maintenance (Encabo, Mateu, Carbonell-Uberos, & Minana, 2003) and in the differentiation of activated B cells into antibody producing plasma cells (Jego et al., 2003; Nurieva et al., 2009). To improve the current NSG-based humanized mice, we generated human IL6 knockin that replaces mouse ortholog in NSGF mice. To this end, we used CRISPR-Cas9 gene targeting in zygotes using Cas9 mRNA, sgRNAs flanking the start codon and stop codon in the mouse Il6 gene and donor plasmid template encompassing 4,308 bp of the human IL6 gene (from the start to stop codons, retaining all exon/intron sequences) flanked by 5′ and 3′ mouse 116 homology sequence. Potential founder mice were selected first with a PCR assay designed specifically against intron 3 and 5 region of human IL6. To determine whether human IL6 was correctly targeted into the murine Il6 locus, we developed long-range PCR assays targeting 5′ and 3′ junctions (with one primer anchored in the mouse genome but outside the donor plasmid homology arms and the other primer anchored within the human IL6 gene) and full-length sequence between two homology arms (expected 8.4 kb in KI, 10.5 kb in wildtype mice) (FIG. 4A). Sequencing of these PCR products confirmed proper targeting of human IL6. In addition, we also confirmed the absence of plasmid donor sequences to discern correct on-target single copy integration events from random or multi-copy targeting events (FIG. 4A). Five founder mice with on-target single copy integration events of human IL6 KI were identified (FIG. 4A). Founders mice with human IL6 knockin (SEQ ID NO: 2) were intercrossed to generate homozygous animals to yield NOD.Cg-Prkdc^{scid} Il2rg^{tm1Wjl} Flt3^{em1Akp}Il6^{em1(IL6)Akp} mouse (NSGF6). To determine whether human IL6 is faithfully expressed, we measured human IL6 production in the serum by ELISA in mice after receiving g LPS IP for 2 hours. We found high level of human IL6 in the serum of mice with IL6^{m/h} and IL6^{h/h} genotype but not IL6^{m/m} (FIG. 4B). One question was whether human IL6 knockin could improve human engraftment after transplantation with different type of HPCs. To evaluate, both NSG and NSGF6 mice were engrafted with titrated amount of cord blood HPCs. While comparable engraftment was found in both strains of mice transplanted with higher number of HPCs, hNSGF6 mice developed higher engraftment when transplanted with low number of HPCs (FIG. 4C). Consistently, hNSGF6 mice transplanted with adult bone marrow HPCs at the limiting number (1×10⁵) demonstrated a significant improvement in hCD45⁺ engraftment in the blood (FIG. 4D). Next, hematopoietic development in hNSGF6 mice was measured. A significantly higher number of total monocytes was found in the spleen and lungs (FIGS. 4E-4F). Furthermore, a higher number of both CD14+CD16+ intermediate, and CD14lowCD16+ non-classical monocytes were found in the spleen and the lungs (FIG. 4G), suggesting that human IL-6 is important for the development of CD16+ monocytes. Furthermore, it was evaluated whether human IL6 knockin improves the differentiation of follicular helper T (Tfh) cells and antibody production in humanized mice. To this end, CXCR5+PD1+CD4+ Tfh cells in the spleen were measured by FACS (FIG. 4H). As shown in FIG. 4I, a significant increase of CXCR5+PD1+CD4+ Tfh cells were found in the spleen of hNSGF6 mice. Consistently with the increase of Tfh, a significantly higher amount of total human IgM, IgG and IgA in the plasma was found (FIG. 4J). In summary, the data demonstrate that humanized mice with human IL6 knockin improves the functional human engraftment upon transplantation of human HPCs.

Generation of Mouse Model: Human IL6 knockin mice (NOD.Cg-Prkdc^{scid}Il2rg^{tmWjl}-Flt3^{em1Akp}Il6^{em1(IL6)Akp)} were generated using CRISPR/cas system. Cas9 mRNA, sgRNAs targeting mouse 116 (5′-AGGAACTTCATAGCGGTTTC-3′, SEQ ID NO: 6 and 5′-ATGCTTAGGCATAACGCACT-3′, SEQ ID NO: 7) and recombinant human IL6 DNA were coinjected into fertilized NSGF oocytes (NOD.Cg-Prkdc^{scid} Il2rg^{tm1Wjl} Flt3^{em1Akp}). Human IL6 was inserted into exon 1 and exon 5 via homologous recombination. The resulting founders, carrying human IL6 were bred to NSGF mice for two generations, and were then interbred until all offspring were homozygous for Il6 targeted mutation. Primer used for genotype by PCR reaction were listed in Table 2.

### Example 3. The NOD.Cg-Prkdc^{scid}Il2rg^{tm1Wjl}-Flt3^{em1Akp}Tslp^{em3(TSLP)Akp} (NSGFT) Mouse Model

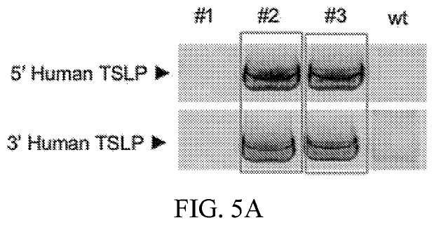

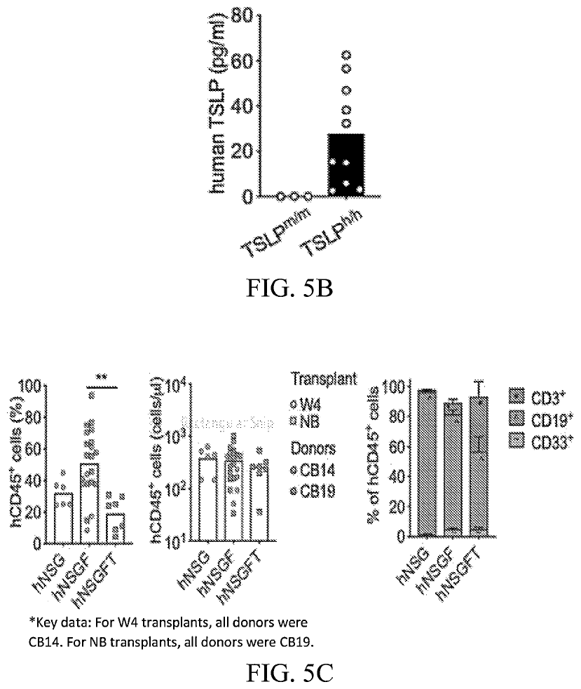

Thymic stromal lymphopoietin (TSLP) is a species-specific cytokine and exhibits species-specific function (Hanabuchi, Watanabe, & Liu, 2012). Human TSLP induces proliferation of naïve T cells, drive Th2 differentiation, Tregs development (Hanabuchi et al., 2010; Ito et al., 2005; Lu et al., 2009). In contrast to IL-7 which directly acts on CD4⁺ T cells, TSLP mediates T cell homeostasis indirectly through human DCs (Lu et al., 2009). To improve the T cell development and differentiation, we generated human TSLP knockin to replace mouse Tslp in NSGF mice. Using CRISPR/cas system, fertilized NSGF oocytes were injected with Cas9 mRNA, sgRNAs flanking the start codon and stop codon in the mouse Tslp gene and donor plasmid template encompassing human TSLP gene (from the start to stop codons, retaining all exon/intron sequences) flanked by 5′ and 3′ mouse Tslp homology sequence. Human TSLP was inserted into exon 1 and exon 5 via homologous recombination. To determine whether human TSLP was correctly targeted into the murine Tslp locus, we developed long-range PCR assays targeting 5′ and 3′ junctions (with one primer anchored in the mouse genome but outside the donor plasmid homology arms and the other primer anchored within the human TSLP gene. Sequencing of these PCR products confirmed proper targeting of human TSLP. Two founder mice with human TSLP KI (SEQ ID NO: 3) were identified (FIG. 5A). The resulting founders, carrying human TSLP were bred to NSGF mice, and were then interbred until all offspring were homozygous for TSLP targeted mutation to yield NOD.Cg-Prkdc^{scid}Il2rg^{tm1Wjl}Flt3^{em1Akp}Tslp^{em3(TSLP)Akp} (NSGFT). To determine whether human TSLP is functional, we measured human TSLP production ex vivo in the supernatant of the lung stimulated with 50 ng/mL of PMA and 1 μg/mL of ionomycin for 18 hours. We found various level of human TSLP production by the lungs of mice with homozygous human TSLP allele but not wt allele (FIG. 5B). To test the effect of TSLP KI on humanization, sublethally irradiated newborn NSGFT mice were transplanted with 1×10⁵ cord blood CD34⁺ HPCs, and the engraftment of human cells was measured in the blood at 12 weeks after transplantation. As shown in FIG. 5C, humanized (h) NSGFT mice allowed a higher reconstitution of human CD3⁺ T cells in the blood while no difference was found on overall hCD45⁺ engraftment.

Generation of Mouse Model: Human TSLP knockin mice (NOD.Cg-Prkdc^{scid}Il2rg^{tm1Wjl}-Flt3^{em1Akp}Tslp^{em3(TSLP)Akp}) were generated using CRISPR/cas system. Cas9 mRNA, sgRNAs targeting mouse Tslp (5′-CCACGTTCAGGCGACAGCAT-3′, SEQ ID NO: 8 and 5′-TTATTCTGGAGATTGCATGA-3′, SEQ ID NO: 9) and recombinant human TSLP DNA were coinjected into fertilized NSGF oocytes (NOD.Cg-Prkdc^{scid} Il2rg^{tm1Wjl}Flt3^{em1Akp}). Human TSLP was inserted into exon 1 and exon 5 via homologous recombination. The resulting founders, carrying human TSLP were bred to NOD.Cg-Prkdc^{scid} Il2rg^{tm1Wjl} Flt3^{em1Akp} mice, and were then interbred until all offspring were homozygous for TSLP targeted mutation. Primer used for genotype by PCR reaction were listed in Table 2.

### Example 4. The NOD.Cg-Prkdc^{scid}Il2rg^{tm1Wjl}-Flt3^{em1Akp}-Ltbr^{em1(LTBR)Akp} (NSGFL) Mouse Model

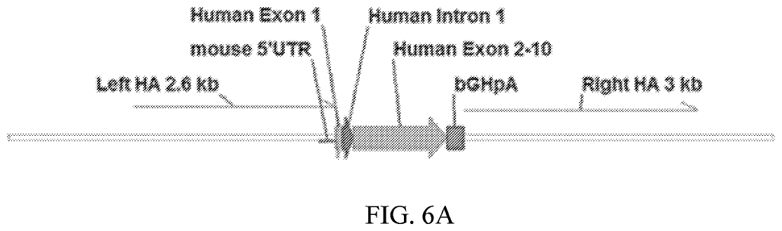

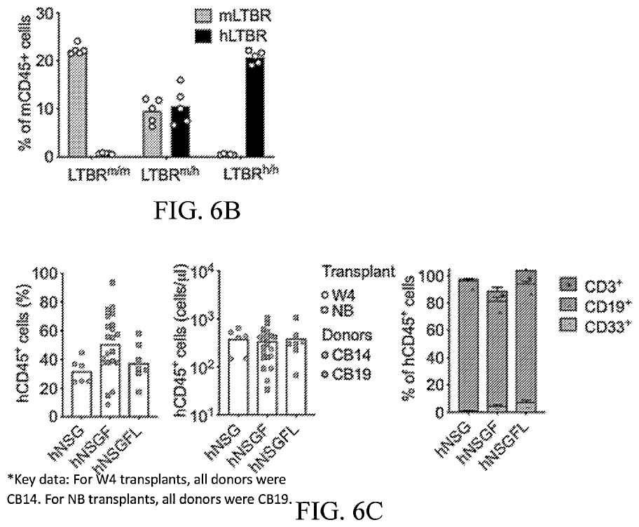

Follicular dendritic cells (FDCs) are essential for the development of lymphoid follicles and B cell responses (Futterer et al., 1998). PDGFRb⁺ Mfge8⁺FDC precursors in the perivascular area of Rag2^{-/-}-γc^{-/-} mice could differentiated into mature FDCs upon the activation of lymphotoxin beta receptor (LTBR) through lymphocyte reconstitution (Krautler et al., 2012). Thus, we generated human LTBR knockin to replace mouse Ltbr in NSGF mice. To this end, we used CRISPR-Cas9 gene targeting in zygotes using Cas9 mRNA, sgRNAs flanking Exon 1 and 2 of mouse Ltbr gene and donor plasmid template encompassing synthetic human LTBR minigene (encodes NM_002342 with all Exon and intron 1 sequences followed by a bGHpA STOP cassette) flanked by 5′ and 3′ mouse Ltbr homology sequence (FIG. 6A). Human LTBR was inserted into exon 1 and exon 2 via homologous recombination. To determine whether human LTBR was correctly targeted into the murine Ltbr locus, we developed long-range PCR assays targeting 5′ and 3′ junctions (with one primer anchored in the mouse genome but outside the donor plasmid homology arms and the other primer anchored within the human LTBR gene) and full-length sequence between two homology arms. Sequencing of these PCR products confirmed proper targeting of human LTBR. Two founder mice with on-target integration events of human LTBR KI (SEQ ID NO: 4) were identified. Founders mice with human LTBR knockin were intercrossed to generate homozygous animals and yield NOD.Cg-Prkdc^{scid}Il2rg^{tm1Wjl}-Flt3^{em1Akp}-Ltbr^{em1(LTBR)Akp} (NSGFL). To determine whether human LTBR is expressed, we measured the surface expression of LTBR in bone marrow cells and observed the expression of human LTBR in mice with LTBR^{m/h} and LTBR^{h/h} but not LTBR^{m/m} (FIG. 6B). To test the effect of LTBR KI on humanization, sublethally irradiated newborn NSGFL mice were transplanted with 1×10⁵ cord blood CD34⁺ HPCs, and the engraftment of human cells was measured in the blood at 12 weeks after transplantation. As shown in FIG. 6C, humanized NSGFL mice allowed a reconstitution of human CD45⁺ immune cells in the blood at 12-wk post-transplant with the differentiation of different immune subsets.

Generation of Mouse Model: Human LTBR knockin mice (NOD.Cg-Prkdc^{scid} Il2rg^{tm1Wjl}-Flt3^{em1Akp}-Ltbr^{em1(LTBR)Akp}) were generated using CRISPR/cas system. Cas9 mRNA, sgRNAs targeting mouse Ltbr (5′-GCTCGGCTGACCAGACCGGG-3′, SEQ ID NO: 10 and 5′-GAGCCACTGTTCTCACCTGG-3′, SEQ ID NO: 11) and synthetic human LTBR minigene (encodes NM_002342 with all Exon and intron 1 sequences followed by a bGHpA STOP cassette) flanked by 5′ and 3′ mouse Ltbr homology sequence were coinjected into fertilized NSGF oocytes (NOD.Cg-Prkdc^{scid} Il2rg^{tm1Wjl} Flt3^{em1Akp}). Human LTBR was inserted into exon 1 and exon 2 via homologous recombination. The resulting founders, carrying human LTBR were bred to NOD.Cg-Prkdc^{scid}Il2rg^{tm1Wjl} Flt3^{em1Akp} mice, and were then interbred until all offspring were homozygous for LTBR targeted mutation. Primer used for genotype by PCR reaction were listed in Table 2.

### Example 5. The NOD.Cg-Prkdc^{scid}Il2rg^{tm1Wjl}-Flt3^{em1Akp} Tg(CMV-IL3,CSF2,KITLG)^{1Eav/MloySzJ} (NSG-SGM3-Flt3KO, SGM3F) Mouse Model

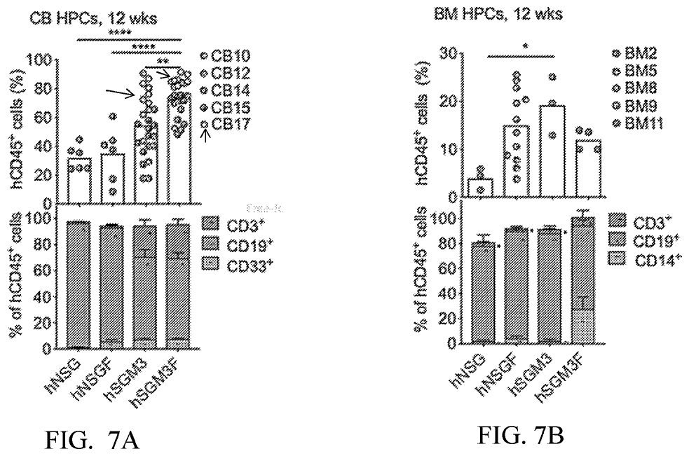

A limited biologic cross-reactivity between murine and human cytokines and cytokine receptors constrains the development of the human innate immune system, especially monocyte, macrophages and neutrophils. Efforts have been made to express human cytokines either through transgenic or knock-in human genes (Rathinam et al., 2011; Rongvaux et al., 2014; Willinger et al., 2011). One such variant of immunodeficient mice is based on NSG mice with transgenic expression of human Stem Cell Factor (SCF), Granulocyte Macrophage-Colony Stimulating Factor (GM-CSF) and Interleukin (IL)-3 (NSG-SGM3, SGM3) (Nicolini et al., 2004; Wunderlich et al., 2010). Initial studies demonstrated that, when transplanted with hCD34⁺ HPCs, these mice efficiently support the development of human immune cells, especially the CD33⁺ myeloid cells as well as CD4⁺Foxp3⁺ regulatory T cells, as compared to non-transgenic counterparts (Billerbeck et al., 2011). To further boost myeloid development, we crossed Flt mutant mice (NSGF) and SGM3 mice to yield NOD.Cg-Prkdc^{scid}Il2rg^{tm1Wjl} Flt3^{em1Akp} Tg(CMV-IL3,CSF2,KITLG)^{1Eav/MloySzJ} (NSG-SGM3-Flt3KO, SGM3F) mice. To test their capacity to support the engraftment of the human immune system, we compared four strains of immunodeficient mice: NSG, NSGF, SGM3, SGM3F mice that were irradiated sub-lethally and transplanted with 1×10⁵ CD34⁺ HPCs from cord blood or from adult bone marrow. While all four strains of mice support cord blood HPCs, hSGM3F mice demonstrate superior hCD45⁺ engraftment at 12-weeks post-transplant with the expansion of both CD33⁺ myeloid cells and CD3⁺ T cells in the blood (FIG. 7A). In contrast, only hNSGF, hSGM3, and hSGM3F mice transplanted with adult bone marrow HPCs at the limiting number (1×10⁵) demonstrated higher hCD45⁺ engraftment in the blood (FIG. 7B). The improvement was particularly pronounced in hSGM3F where we observed a higher percentage of CD14⁺ monocytes and CD3⁺ T cells in the blood (FIG. 7B).

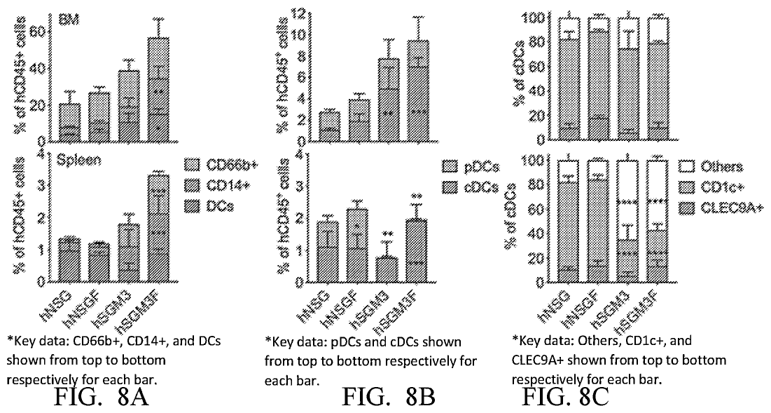

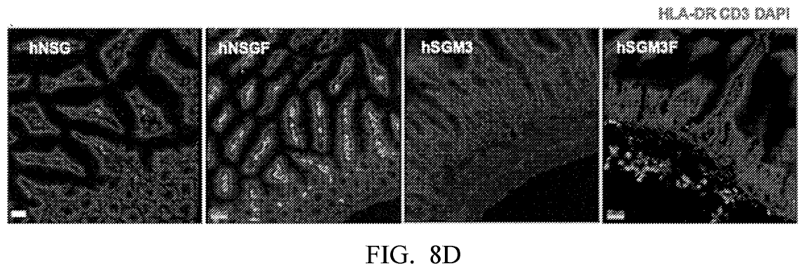

Next, we compared the development of myeloid compartment including CD66b⁺ granulocytic, CD14⁺ monocytic myeloid cells and DCs in different strains of humanized mice. While all four strains of mice support the differentiation of different myeloid cells in the bone marrow, hSGM3F mice demonstrate higher expansion of CD14⁺ and DCs (FIG. 8A). More importantly, we observed a higher percentage of both CD14⁺ and CD66b⁺ cells in the spleen (FIG. 8A). Of note, the CD66b⁺ cells were absent in the hNSG and hNSGF suggesting an important role of human SCF/GM-CSF/IL-3 cytokines on their development. Human DCs constituted CD303⁺ pDCs and CD11c cDCs, which were further divided into CD1c+ or CLEC9A+ subsets. The analysis of human DC development revealed a significant increase of cDCs in the mouse bone marrow of both hSGM3 and hSGM3F mice whereas a significant decrease in pDCs and increase in cDCs were observed in the spleen of hSGM3F mice (FIG. 8B). Since pDCs were significantly increased in hNSGF but decreased in hSGM3, the decrease in pDCs in hSGM3F can be largely attributed to human IL3/CSF2/KITLG transgenes, which promote development of immature myeloid cells at the expense of other cell subsets. A similar effect was observed in the subsets of cDCs with a smaller proportion of classical CD1c cDCs found in the spleen of both hSGM3 and hSGM3F mice than hNSG or hNSGF mice (FIG. 8C). In addition, we also observed colonization of mucosal tissues with human DCs by the presence of HLA-DR⁺ cells in the lamina propria of the small intestine with the morphology of DCs in hNSGF and hSGM3F mice (FIG. 8D). Overall, our data suggest a biological effect of mouse Flt3 KO and human IL3/CSF2/KITLG transgenes on human DC development in humanized mice.

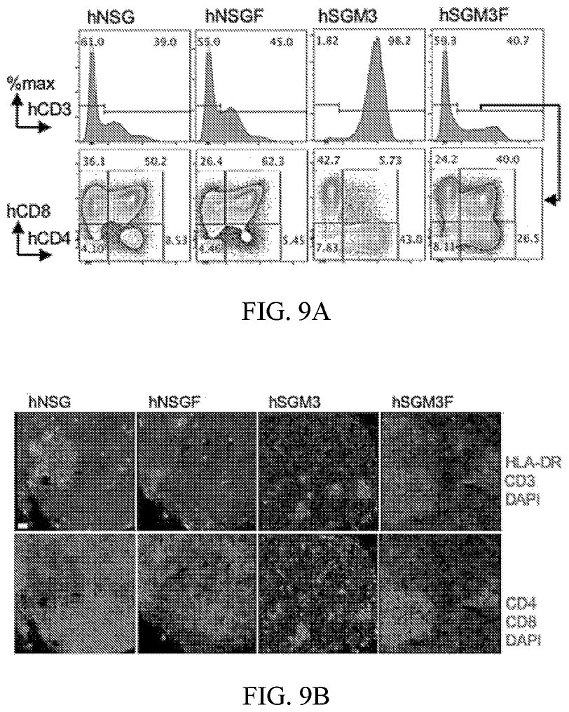

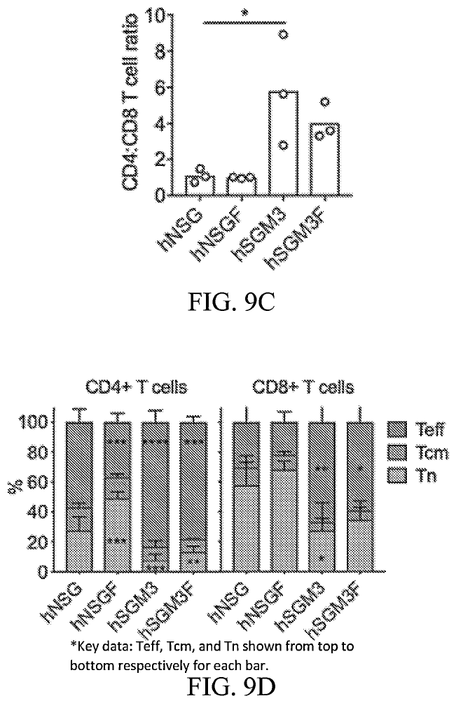

In thymus, the majority of human CD3⁺ thymocytes were double-positive for CD4 and CD8 in hNSG and hNSGF mice, while higher percentage of single-positive CD4 or CD8 thymocytes were found in both hSGM3 and hSGM3F mice (FIG. 9A-9B). This suggests a potential higher output of mature thymocytes, which is consistent with us finding more CD3⁺ T cells in the blood of hSGM3 and hSGM3F mice (FIG. 7). At 20 weeks post-transplant, we observed in the spleen a slightly higher CD4:CD8 T cell ratio in both hSGM3 and hSGM3F mice (FIG. 9C). Significantly, the proportion of CD45RA^{+/-}CCR7⁻ effector T cells was decreased in hNSGF mice but increased in hSGM3 and hSGM3F mice for both CD4⁺ T cells and CD8⁺ T cells, although to a lesser degree in the latter (FIG. 9D). Consequently, the proportion of CD45RA⁺CCR7⁺ naive T cells was largely decreased in hSGM3 and hSGM3F mice (FIG. 9D). Overall, our data suggest a superior human engraftment in SGM3F mice.

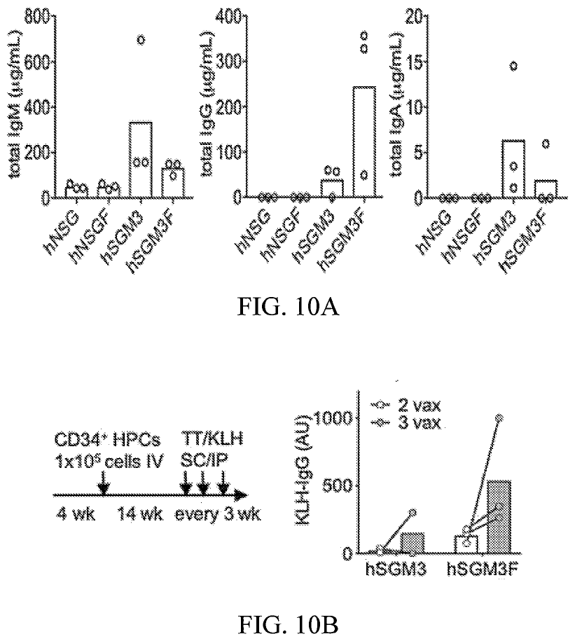

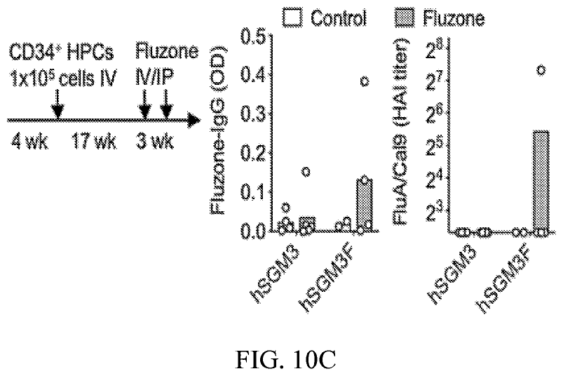

Finally, we sought to probe the capacity of humanized mouse strains to mount antibody responses to vaccination. We first measured the level of different immunoglobulin (Ig) isotype in the plasma of humanized mice. hNSG and hNSGF had little human IgG and IgA in the plasma while hSGM3 and hSGM3F had higher level of different Ig isotypes (FIG. 10A), suggesting the capacity for efficient Ig class-switch and T cell dependent response. Next, we vaccinated different strains of humanized mice with alum-adjuvanted Tdap/KLH vaccine IP/SC at 17-, 20-, and 23-weeks post-transplant (FIG. 10B). Three out of three vaccinated mice developed specific IgG to KLH in hSGM3F mice, and that the specific antibody remained detectable at 6 weeks after the 3^{rd} vaccination (FIG. 10B). Furthermore, we vaccinated additional mice with Fluzone IV/IP with 1/10^{th} of the human dose at 17- and 20-weeks post-transplant. At 10-days post 2^{nd} vaccination, we observed that two out of four vaccinated mice developed specific IgG to Fluzone in hSGM3F mice (FIG. 10C). More importantly, one out of four hSGM3F mice developed neutralizing antibody to one of the vaccine strains, H1N1 FluA/Cal9 virus, but not to influenza B virus as measured by hemagglutination inhibition assay (FIG. 10C). In summary, our data indicate a significant functional improvement of the human immune system in hSGM3F mice.

Generation of Mouse Model: NSG-SGM3-Flt3ko or SGM3F mice (NOD.Cg-Prkdc^{scid}Il2rg^{tm1Wjl}-Flt3^{em1Akp} Tg(CMV-IL3, CSF2,KITLG)^{1Eav/MloySzJ}), were generated by crossing NSG-SGM3 mice (NOD.Cg-Prkdc^{scid}Il2rg^{tm1Wjl} Tg(CMV-IL3,CSF2,KITLG)^{1Eav/MloySzJ}; RRID:IMSR Jackson Lab Stock #013062) to NSGF mice and interbred until all offspring were homozygous. NSG-SGM3 mice carried three separate transgenes which were designed each carrying either the human interleukin-3 (IL-3) gene, the human granulocyte/macrophage-stimulating factor (GM-CSF) gene, or human Steel factor (SF) gene. Expression of each gene is driven by a human cytomegalovirus promoter/enhancer sequence, and is followed by a human growth hormone cassette and a polyadenylation (polyA) sequence. The transgenes were microinjected into fertilized C57BL/6×C3H/HeN oocytes. The resulting founders, carrying all three transgenes (3GS) were backcrossed to BALB/c-scid/scid mice for several generations and subsequently backcrossed to NOD.CB17-Prkdc^{scid} mice for at least 11 generations. These mice were bred to NSG mice (NOD.Cg-Prkdc^{scid} Il2rg^{tm1Wjl}; RRID:IMSR JAX: 005557), and were then interbred until all offspring were homozygous for 3GS and the IL2rg targeted mutation. Upon arrival at The Jackson Laboratory, transgenic mice were bred to NSG mice for one generation to establish NSG-SGM3 mice. NSGF mice were generated using CRISPR/cas system. Cas9 mRNA and sgRNAs targeting mouse Flt3 were coinjected into fertilized NSG oocytes. The resulting founders, carrying Flt3 deletion were bred to NSG mice, and were then interbred until all offspring were homozygous for Flt3 targeted mutation.

TABLE 1
List of mouse strains.

| Name | Abbreviation | Features |
|---|---|---|
| NOD.Cg-Prkdc^{scid} Il2rg^{tm1Wjl}-Flt3^{em1Akp} | NSGF | Mouse Flt3 knockout |
| NOD.Cg-Prkdc^{scid} Il2rg^{tm1Wjl}-Flt3^{em1Akp}Il6^{em1(IL6)Akp} | NSGF6 | Mouse Flt3 knockout |
| | | and human IL6 knockin |
| NOD.Cg-Prkdc^{scid} Il2rg^{tm1Wjl}-Flt3^{em1Akp}Tslp^{em3(TSLP)Akp} | NSGFT | Mouse Flt3 knockout |
| | | and human TSLP knockin |
| NOD.Cg-Prkdc^{scid} Il2rg^{tm1Wjl}-Flt3^{em1Akp} -Ltbr^{em1(LTBR)Akp} | NSGFL | Mouse Flt3 knockout |
| | | and human LTBR knockin |
| NOD.Cg-Prkdc^{scid} Il2rg^{tm1Wjl}-Flt3^{em1Akp} | SGM3F | Mouse Flt3 knockout |
| Tg(CMV-IL3, CSF2, KITLG)^{1Eav/MloySzJ} | | and human IL3, CSF2, |
| | | KITLG transgenic |

TABLE 2
List of PCR primers for mouse genotype.

| Allele | Name | Sequence |
|---|---|---|
| Flt3 | mFlt3_F | GGTACCAGCAGAGTTGGATAGC (SEQ ID NO: 12) |
| Flt3 | mFlt3_R | ATCCCTTACACAGAAGCTGGAG (SEQ ID NO: 13) |
| IL6 | IL6_5′_F, | CATCTCCTGTGGGACCATTCTTC (SEQ ID NO: 14) |
| | IL6_full_F | |
| IL6 | IL6_5′_R | AGTGCAGGTTATCTCACTGTGG (SEQ ID NO: 15) |
| IL6 | IL6_3′_F | TTGGAACTGAACCCAAGTGTGC (SEQ ID NO: 16) |
| IL6 | IL6_3′_R, | GGCTGTCCTCAGACCCAATC (SEQ ID NO: 17) |
| | IL6_full_R | |
| IL6 | IL6_backbone_F | GAAGTTTGTTGCTATGGAAGGGTC (SEQ ID NO: 18) |
| IL6 | IL6_backbone_R | AGCGCAACGCAATTAATGTG (SEQ ID NO: 19) |
| TSLP | TSLP_5′_F | CCTTCTCGTGTGAATAAGCTGC (SEQ ID NO: 20) |
| TSLP | TSLP_5′_R | CTCATCAGCATCTGCACACTTAG (SEQ ID NO: 21) |
| TSLP | TSLP_3′_F | CAGGGAGGTCTTGAAATCAGC (SEQ ID NO: 22) |
| TSLP | TSLP_3′_R | CCAGGCTGTAGCATTTGGGTG (SEQ ID NO: 23) |
| LTBR | LTBR 5′ F | GTGAAATGTATCTAGGGCCGCTC (SEQ ID NO: 24) |
| LTBR | LTBR_5′_R | TGCTCTGTCTCCGCTAGGTG (SEQ ID NO: 25) |
| LTBR | LTBR_3′_F | AGAGGTTCAGAGTTGTTCTCAGG (SEQ ID NO: 26) |
| LTBR | LTBR_3′_R | ATGCGTCGGAGAACCAGACC (SEQ ID NO: 27) |

Additional Materials and Methods

Humanized Mice

Humanized mice were generated on different strains of mice in NSG background obtained from The Jackson Laboratory (Bar Harbor, ME). All protocols were reviewed and approved by the Institutional Animal Care and Use Committee at The Jackson Laboratory (14005) and University of Connecticut Health Center (101163-0220 & 101831-0321; Farmington, CT). Mice were sub-lethally irradiated (10 cGy per gram of body weight) using gamma irradiation at the age of four weeks. 100,000 CD34⁺ HPCs from fetal liver or full-term cord blood (Advanced Bioscience Resources or Lonza) were given by tail-vein intravenous (IV) injection in 200 μL of PBS. Alternatively, mice received adult CD34⁺ HPCs from bone marrow (Lonza) as indicated. Mice were bled at 4-12 weeks post HPC transplant to evaluate engraftment and euthanized according to the individual experimental design.

### Flow Cytometry Analysis

Mice were euthanized and blood was collected with heparin. The bones (femur and tibia), spleen and lungs were collected to make single cell suspension. Spleen were digested with 50 μg/ml of Liberase (Roche Diagnostics, Indianapolis, IN) and 24 U/mL of DNase I (Sigma) for 10 min at 37° C. Lungs were digested with 50 μg/ml of Liberase and 24 U/mL of DNase I (Sigma) for 30 min at 37° C., followed by mechanical dissociation with GentleMACS (Miltenyi Biotec). Cells were first treated with murine Fc blocker (BD) and then stained on ice with antibody cocktails for 30 mins. After washing twice with PBS, the samples were acquired on a LSRII or FACSARIA II (BD), and analyzed with FlowJo software (Tree Star, Ashland, OR). For the expression of mouse Flt3, cells were stained with antibodies to mouse CD45-BV650 (30⁻ F11, BD) and FLT3-BV421 (A2F10.1, BD). For the expression of human LTBR, cells were stained with antibodies to mouse CD45-BV650 (30-F11, BD) and human LTBR-PE (31G4D8, BD). For the analysis of mouse DCs, cells were stained antibodies to mouse CD45-BV650 (30⁻ F11, BD), CD3-PE-CF579 (145-2C11, BD), CD19-PE-CF579 (ID3, BD), CD103-PerCP-Cy5.5 (M290, BD), F4/80-PE-Cy7 (F4/80, BD), Gr1-PO (RB6-8C5, BD), IAg7-FITC (10-2-16, BD), CD11c-V450 (HL3, BD), CD172a-PE (P84, BD), CD8-PE (53-6.72, BD), and PDCA-1-APC (927, Biolegend). For human engraftment in the blood, cells were stained with antibodies to mouse CD45-BV650 (30-F11, BD) and human CD45-BV510 (HI30, BD), CD33-PE (P67.6, Biolegend), CD14-PE-Cy7 (MqP9, BD), CD19-APC (HIB19, Biolegend) and CD3-APC-H7 (SK7, BD). For human immune cell phenotype, additional antibodies were used to stain bone marrow, spleen and thymus including antibodies to human CD1c-PerCPCy5.5 (L161, Biolegend), CLEC9A-PE (8F9, Biolegend), CD303-FITC (AC144, Miltenyi Biotec), HLA-DR-APC-eFour 780 (LN3, Thermofisher), CD11c-V450 (B-ly6, BD), CD66b-FITC (G10F5, BD), CD8-ECD (SF121Thy2D3, Beckman Coulter), CD4-BUV395 (SK3, BD), CD45RA-PerCPCy5.5 (HI100, BD) and CCR7-PE-Cy7 (3D12, BD).

### Immunofluorescence Staining

Tissues were embedded in OCT (Sakura Finetek U.S.A.) and snap frozen in liquid nitrogen. Frozen sections were cut at 6 μm, air dried on Superfrost plus slides and fixed with cold acetone for five minutes. Tissue sections were first treated with 0.03% hyaluronidase (Sigma) for 15 minutes, followed by treatment with Background Buster and Fc Receptor Block (Innovex Bioscience). The sections were then stained with monoclonal antibodies to mouse I-A^{g7} (10.2.16, BD), human CD3 (UCHT1, Biolegend), CD4 (RPA-T4, Biolegend), CD8 (RPA-T8, BD), CD11c (S-HCL-3, BD), or HLA-DR (L243, Biolegend) for one hour at room temperature, followed by isotype-specific secondary antibodies for 30 minutes at room temperature. Respective isotype antibodies were used as the control. Finally, sections were counterstained with 1 μg/ml of 4′,6-diamidino-2-phenylindole (DAPI), mounted with Fluoromount (Thermo Fisher Scientific), and visualized using a Leica SP 8 confocal microscope with Leica LAS AF 2.0 software or a Zeiss Axio fluorescence microscope with ZEN software.

### ELISA

Cytokine production were measured with ELISA kit following manufacture protocol. For mouse Flt3L, plasma from both WT and Flt3-KO mice were tested with mouse Flt3L ELISA Duo Set from R&D systems. For human IL6, plasma from both WT and IL6-KI mice treated with 20 μg of LPS (Invivogen) IP for 2 hours were tested with human IL6 ELISA MAX Deluxe Set from Biolegend. For human TSLP, mouse lungs from both WT and TSLP-KI mice were stimulated ex-vivo with 50 ng/mL of PMA (Sigma) and 1 μg/mL of ionomycin (Sigma) for 18 hours and human TSLP were measured in the culture supernatant with human TSLP ELISA Max Deluxe Set from Biolegend. For total human IgM, IgG and IgA, plasma samples were tested with Human IgM, IgG, and IgA ELISA kit (Bethyl Laboratories). For KLH-specific human IgG, ELISA plates were coated with 10 μg/mL of purified KLH (Thermo Fisher Scientific) and detected with Human IgG ELISA kit. For Fluzone-specific human IgG, ELISA plates were coated with Fluzone (2015-2016 season, Sanofi) and detected with Human IgG ELISA kit.

### Hemagglutination Inhibition Assay

The hemagglutination inhibition (HAI) assay was performed to detect and quantitate antiviral antibodies in the serum. Aliquots of 50 μl of serum (including all the test sera and reference human serum as positive control) were first treated with receptor destroying enzyme (Sigma) for 16-18 hours at 37° C. Sera were then heated to 56° C. for thirty minutes to remove the enzyme activity and incubated with 200 μl of 1% chicken red blood cells (CRBCs) at room temperature for thirty minutes to remove non-specific hemagglutination activity in the serum. Diluted samples (⅕ dilution) were recovered by centrifuging at 1200 rpm for ten minutes. Mixture of 50 μl of influenza virus containing 4 HA unit and 50 μl of 2-fold serial diluted serum are incubate at room temperature for thirty minutes in duplicate on 96-well U bottom plates. Then, 50 μl of 1% CRBCs were added into each well and incubated at room temperature for forty-five minutes. The HAI titer was defined as the reciprocal of the final dilution that does not give hemagglutination.

### Statistical Analysis

Statistical analyses were performed in Prism (GraphPad). Comparisons between any 2 groups were analyzed using the Mann-Whitney test or two-tailed t-test. Comparisons between any 3 or more groups were analyzed by analysis of variance (ANOVA).

SEQUENCES

SEQ ID NO: 1, Flt^{3em1Akp}
GGGCACGTGGGATCGGCTGCAGCACTGCGCCAGTTCAGCCCGCCTAGCAGCGAGCG
GCCGCGGCCTCTGGAGAGAGGTTCCTCCCCCTCTGCTCTGCACCAGTCCGAGGGAAT
CTGTGGTCAGTGACGCGCATCCTTCAGCGAGCCACCTGCAGCCCGGGGCGCGCCGC
TGGGACCGCATCACAGGCTGGGCCGGCGGCCTGGCTACCGCGCGCTCCGGAGGCCA
TGCGGGCGTTGGCGCAGCGCAGCGACCGGCGGCTGCTGCTGCTTGTTGTTTTGTCAG
TAATGATTCTTGAGACCGTTACAAACCAAGACCTGCCTGTGATCAAGTGTGTTTTAA
TCAGTCATGAGAACAATGGCTCATCAGCGGGAAAGCCATCATCGTACCGAATGAGG
AATCGTTTCCATGGCCATCTTGAACGTGACAGAGACCCAGGCAGGAGAATACCTAC
TCCATATTCAGAGCGAAGCCGCCAACTACACAGTACTGTTCACAGTGAATGTAAGA
GATACACAGCTGTACGTGCTAAGAAGACCTTACTTTAGGAAGATGGAAAACCAGGA
CGCACTGCTCTGCATCTCCGAGGGTGTTCCAGAGCCCACTGTGGAGTGGGTGCTCTG
CAGCTCCCACAGGGAAAGCTGTAAAGAAGAAGGCCCTGCTGTTGTCAGAAAGGAG
GAAAAGGTACTTCATGAGTTGTTCGGAACAGACATCAGATGCTGTGCTAGAAATGC
ACTGGGCCGCGAATGCACCAAGCTGTTCACCATAGATCTAAACCAGGCTCCTCAGA
GCACACTGCCCCAGTTATTCCTGAAAGTGGGGGAACCCTTGTGGATCAGGTGTAAG
GCCATCCATGTGAACCATGGATTCGGGCTCACCTGGGAGCTGGAAGACAAAGCCCT
GGAGGAGGGCAGCTACTTTGAGATGAGTACCTACTCCACAAACAGGACCATGATTC
GGATTCTCTTGGCCTTTGTGTCTTCCGTGGGAAGGAACGACACCGGATATTACACCT
GCTCTTCCTCAAAGCACCCCAGCCAGTCAGCGTTGGTGACCATCCTAGAAAAAGGG
TTTATAAACGCTACCAGCTCGCAAGAAGAGTATGAAATTGACCCGTACGAAAAGTT
CTGCTTCTCAGTCAGGTTTAAAGCGTACCCACGAATCCGATGCACGTGGATCTTCTC
TCAAGCCTCATTTCCTTGTGAACAGAGAGGCCTGGAGGATGGGTACAGCATATCTA
AATTTTGCGATCATAAGAACAAGCCAGGAGAGTACATATTCTATGCAGAAAATGAT
GACGCCCAGTTCACCAAAATGTTCACGCTGAATATAAGAAAGAAACCTCAAGTGCT
AGCAAATGCCTCAGCCAGCCAGGCGTCCTGTTCCTCTGATGGCTACCCGCTACCCTC
TTGGACCTGGAAGAAGTGTTCGGACAAATCTCCCAATTGCACGGAGGAAATCCCAG
AAGGAGTTTGGAATAAAAAGGCTAACAGAAAAGTGTTTGGCCAGTGGGTGTCGAGC
AGTACTCTAAATATGAGTGAGGCCGGGAAAGGGCTTCTGGTCAAATGCTGTGCGTA
CAATTCTATGGGCACGTCTTGCGAAACCATCTTTTTAAACTCACCAGGCCCCTTCCC
TTTCATCCAAGACAACATCTCCTTCTATGCGACCATTGGGCTCTGTCTCCCCTTCATT
GTTGTTCTCATTGTGTTGATCTGCCACAAATACAAAAAGCAATTTAGGTACGAGAGT
CAGCTGCAGATGATCCAGGTGACTGGCCCCCTGGATAACGAGTACTTCTACGTTGAC
TTCAGGGACTATGAATATGACCTTAAGTGGGAGTTCCCGAGAGAGAACTTAGAGTT
TOGGAAGGTCCTGGGGTCTGGCGCTTTCGGGAGGGTGATGAACGCCACGGCCTATG
GCATTAGTAAAACGGGAGTCTCAATTCAGGTGGCGGTGAAGATGCTAAAAGAGAAA
GCTGACAGCTGTGAAAAAGAAGCTCTCATGTCGGAGCTCAAAATGATGACCCACCT
GGGACACCATGACAACATCGTGAATCTGCTGGGGGCATGCACACTGTCAGGGCCAG
TGTACTTGATTTTTGAATATTGTTGCTATGGTGACCTCCTCAACTACCTAAGAAGTA
AAAGAGAGAAGTTTCACAGGACATGGACAGAGATTTTTAAGGAACATAATTTCAGT
TTTTACCCTACTTTCCAGGCACATTCAAATTCCAGCTTCAGAATGAATTAAATTCCC
ATTGAACCCTGAGAGCTGATCCAAGGGCGGGTGTAACTGAACTTCTCGTGAACCAG
GCATGATGAGATTGAATATGAAAACCAGAAGAGGCTGGCAGAAGAAGAGGAGGAA
GATTTGAACGTGCTGACGTTTGAAGACCTCCTTTGCTTTGCGTACCAAGTGGCCAAA
GGCATGGAATTCCTGGAGTTCAAGTCGTGTGTCCACAGAGACCTGGCAGCCAGGAA
TGTGTTGGTCACCCACGGGAAGGTGGTGAAGATCTGTGACTTTGGACTGGCCCGAG
ACATCCTGAGCGACTCCAGCTACGTCGTCAGGGGCAACGCACGGCTGCCGGTGAAG
TGGATGGCACCTGAGAGCTTATTTGAAGGGATCTACACAATCAAGAGTGACGTCTG
GTCCTACGGCATCCTTCTCTGGGAGATATTTTCACTGGGTGTGAACCCTTACCCTGG
CATTCCTGTCGACGCTAACTTCTATAAACTGATTCAGAGTGGATTTAAAATGGAGCA
GCCATTCTATGCCACAGAAGGGATATGTATCAGAACATGGGTGGCAACGTCCCAGA
ACATCCATCCATCTACCAAAACAGGCGGCCCCTCAGCAGAGAGGCAGGCTCAGAGC
CGCCATCGCCACAGGCCCAGGTGAAGATTCACGGAGAAAGAAGTTAGCGAGGAGG
CCTTGGACCCCGCCACCCTAGCAGGCTGTAGACCACAGAGCCAAGATTAGCCTCGC
CTCTGAGGAAGCGCCCTACAGGCCGTTGCTTCGCTGGACTTTTCTCTAGATGCTGTC
TGCCATTACTCCAAAGTGACTTCTATAAAATCAAACCTCTCCTCGCACAGGTGGGAG
AGCCAATAATGAGACTTGTTGGTGAGCCCGCCTACCCTGGGGGGCCTTTCCAGGCCC
CCCAGGCTTGAGGGGAAAGCCATGTATCTGAAATATAGTATATTCTTGTAAATACGT
GAAACAAACCAAACCCGTTTTTTGCTAAGGGAAAGCTAAATATGATTTTTAAAAAT
CTATGTTTTAAAATACTATGTAACTTTTTCATCTATTTAGTGATATATTTTATGGATG
GAAATAAACTTTCTACTGTAGAAA
SEQ ID NO: 2, Il6^{em1(IL6)Akp}
TCATGGATGTATGCTCCCGACTTAAAAAGCACCTTTTTTAAAAAACTAAAAACAGA
AATCTGAATGTTGTAGTAAGTGTAACAATCTTAAGTTTATTCAGTAATTTAAAAAAA
TTGTTAAGCGGAGAAAAGAAACTCTGTACTAACAGAGGCCTGAGAAAGCACACGGC
AGGGAATAGGGGAAATGGCTTCCTTCATTGCTGGACACAGACTGAGCTCCAGGCTG
TTTCAGCTGCCTTTTTAAGGCTCAAGGGCACTAAAAGTAAAACCATCCTGCTTCCTC
TCCCCATTTTCATTTTCACCTAAAATCCCCTAGTCCCTTTGTGAAGACCAGGGCTTCA
CACGGTGAAAGAATGGTGGACTCACTTCTTTCAATAGGCTGACCTAGTATGTACACT
AAGTCCACCCATGTTTTAACTTTCTTCCTAGTTTATTCCCCTTCTGATTTCTTCACAA
GAATCAACCGGCTTTTCATTTTAATCTACTCTAATCGCCTGTGTGTTTACACTGGGTT
ACATTCTTTAGAGTGTACTTATATTCTCCTTTTGCATTCTCAATATAAATTAATCTGC
TAGATATAAAGCTGTTCTCTTTATTTTAGTGTAATTTTTTTCTTCACATTGAATTCTA
GGAGAAACTATGCTAGTGATATATAATTCTTGAACTATTAAACATGGGAGCATAAG
AAAACAAGAATCTTAAGGCAATCTGCAGAGTGAAGAAGCTGATTGTGATCCTGAGA
GTGTGTTTTGTAAATGGTTTTGGATTTTATGTACAGAGCCTACTTTCAGCCTGGAATC
ATTCTGAATGCTAGCTAGATATCTGGAGACAGGTGGACAGAAAACCAGGAACTAGT
CTGAAAAAGAAACTAACCAAAGGGAAGAAGTCTGTTTAAGTTTGACCCAGCCTAGA
AGACTTGAGCATTGGAGGGGTTATTCAGAGTGAGACGTACCACCTTCAGATTCAAA
TCCTGTCATCCAGTAGAAGGGAGCTTCAAACACAAGCTAGCTAAGATACAATGAGG
TCCTTCTTCGATATCTTTATCTTCCATATACCATGAATCAAAGAAACTTCAACAACAT
GAGGACTGCAACAGACCTTCAAGCCTCCTTGCATGACCTGGAAATGTTTTGGGGTGT
CCTGGCAGCAGTGGGATCAGCACTAACAGATAAGGGCAACTCTCACAGAGACTAAA
GGTCTTAACTAAGAAGATAGCCAAGAGACCACTGGGGAGAATGCAGAGAATAGGC
TTGGACTTGGAAGCCAAGATTGCTTGACAACAGACAGAAGATATTTCTGTACTTCAC
CCACTTTACCCACCTGGCAACTCCTGGAAACAACTGCACAAAATTTGGAGGTGAAC
AAACCATTAGAAACAACTGGTCCTGACAAGACACAGGAAAAACAAGCAATATGCA
ACATTACTGTCTGTTGTCCAGGTTGGGTGCTGGGGGTGGGAGAGGGAGTGTGTGTCT
TTGTATGATCTGAAAAAACTCAGGTCAGAACATCTGTAGATCCTTACAGACATACA
AAAGAATCCTAGCCTCTTATTCACGTCTGTCATGCGCGCGTGCCTGCGTTTAAATAA
CATCAGCTTTAGCTTCTCTTTCTCCTTATAAAACATTGTGAATTTCAGTTTTCTTTCCC
ATCAAGACATGCTCAAGTGCTGAGTCACTTTTAAAGGAAGAGTGCTCATGCTTCTTA
GGGCTAGCCTCAAGGATGACTTAAGCACACTTTCCCCTTCCTAGTTGTGATTCTTTC
GATGCTAAACGACGTCACATTGTGCAATCTTAATAAGGTTTCCAATCAGCCCCACCC
ACTCTGGCCCCACCCCCACCCTCCAACAAAGATTTTTATCAAATGTGGGATTTTCCC
ATGAGTCTCAAAATTAGAGAGTTGACTCCTAATAAATATGAGACTGGGGATGTCTG
TAGCTCATTCTGCTCTGGAGCCCACCAAGAACGATAGTCAATTCCAGAAACCGCTAT
GAACTCCTTCTCCACAAGTAAGTGCAGGAAATCCTTAGCCCTGGAACTGCCAGCGG
CGGTCGAGCCCTGTGTGAGGGAGGGGTGTGTGGCCCAGGGAGGGCTGGCGGGCGG
CCAGCAGCAGAGGCAGGCTCCCAGCTGTGCTGTCAGCTCACCCCTGCGCTCGCTCCC
CTCCGGCACAGGCGCCTTCGGTCCAGTTGCCTTCTCCCTGGGGCTGCTCCTGGTGTT
GCCTGCTGCCTTCCCTGCCCCAGTACCCCCAGGAGAAGATTCCAAAGATGTAGCCGC
CCCACACAGACAGCCACTCACCTCTTCAGAACGAATTGACAAACAAATTCGGTACA
TCCTCGACGGCATCTCAGCCCTGAGAAAGGAGGTGGGTAGGCTTGGCGATGGGGTT
GAAGGGCCCGGTGCGCATGCGTTCCCCTTGCCCCTGCGTGTGGCCGGGGGCTGCCTG
CATTAGGAGGTCTTTGCTGGGTTCTAGAGCACTGTAGATTTGAGGCCAACGGGGCC
GACTAGACTGACTTCTGTATTTATCCTTTGCTGGTGTCAGGAGTTCCTTTCCTTTCTG
GAAAATGCAGAATGGGTCTGAAATCCATGCCCACCTTTGGCATGAGCTGAGGGTTA
TTGCTTCTCAGGGCTTCCTTTTCCCTTTCCAAAAAATTAGGTCTGTGAAGCTCCTTTT
TGTCCCCCGGGCTTTGGAAGGACTAGAAAAGTGCCACCTGAAAGGCATGTTCAGCT
TCTCAGAGCAGTTGCAGTACTTTTTGGTTATGTAAACTCAATGGTAGGATTCCTCAA
AGCCATTCCAGCTAAGATTCATACCTCAGAGCCCACCAAAGTGGCAAATCATAAAT
AGGTTAAAGCATCTCCCCACTTTCAATGCAAGGTATTTTGGTCCTGTTGGCTTGAAT
TATATTCTCCTAATTATTGTCAAAATTGCTGACTGGAATTTGCTTGCCAGGATGCCA
ATGAGTTGTAGCTTCATTTTTCTTAGAGACTTTCCTGGCTGTGGTTGAACAATGAAA
AGGCCCTCTAGTGGTGTTTGTTTTAGGGACACTTAGGTGATAACAATTCTGGTATTC
TTTCCCAGACATGTAACAAGAGTAACATGTGTGAAAGCAGCAAAGAGGCACTGGCA
GAAAACAACCTGAACCTTCCAAAGATGGCTGAAAAAGATGGATGCTTCCAATCTGG
ATTCAATGAGGTACCAACTTGTCGCACTCACTTTTCACTATTCCTTAGGCAAAACTT
CTCCCTCTTGCATGCAGTGCCTGTATACATATAGATCCAGGCAGCAACAAAAAGTG
GGTAAATGTAAAGAATGTTATGTAAATTTCATGAGGAGGCCAACTTCAAGCTTTTTT
AAAGGCAGTTTATTCTTGGACAGGTATGGCCAGAGATGGTGCCACTGTGGTGAGAT
TTTAACAACTGTCAAATGTTTAAAACTCCCACAGGTTTAATTAGTTCATCCTGGGAA
AGGTACTCTCAGGGCCTTTTCCCTCTCTGGCTGCCCCTGGCAGGGTCCAGGTCTGCC
CTCCCTCCCTGCCCAGCTCATTCTCCACAGTGAGATAACCTGCACTGTCTTCTGATTA
TTTTATAAAAGGAGGTTCCAGCCCAGCATTAACAAGGGCAAGAGTGCAGGAAGAAC
ATCAAGGGGGACAATCAGAGAAGGATCCCCATTGCCACATTCTAGCATCTGTTGGG
CTTTGGATAAAACTAATTACATGGGGCCTCTGATTGTCCAGTTATTTAAAATGGTGC
TGTCCAATGTCCCAAAACATGCTGCCTAAGAGGTACTTGAAGTTCTCTAGAGGAGC
AGAGGGAAAAGATGTCGAACTGTGGCAATTTTAACTTTTCAAATTGATTCTATCTCC
TGGCGATAACCAATTTTCCCACCATCTTTCCTCTTAGGAGACTTGCCTGGTGAAAAT
CATCACTGGTCTTTTGGAGTTTGAGGTATACCTAGAGTACCTCCAGAACAGATTTGA
GAGTAGTGAGGAACAAGCCAGAGCTGTGCAGATGAGTACAAAAGTCCTGATCCAGT
TCCTGCAGAAAAAGGTGGGTGTGTCCTCATTCCCTCAACTTGGTGTGGGGGAAGAC
AGGCTCAAAGACAGTGTCCTGGACAACTCAGGGATGCAATGCCACTTCCAAAAGAG
AAGGCTACACGTAAACAAAAGAGTCTGAGAAATAGTTTCTGATTGTTATTGTTAAAT
CTTTTTTTGTTTGTTTGGTTGGTTGGCTCTCTTCTGCAAAGGACATCAATAACTGTAT
TTTAAACTATATATTAACTGAGGTGGATTTTAACATCAATTTTTAATAGTGCAAGAG
ATTTAAAACCAAAGGCGGGGGGGCGGGCAGAAAAAAGTGCATCCAACTCCAGCCA
GTGATCCACAGAAACAAAGACCAAGGAGCACAAAATGATTTTAAGATTTTAGTCAT
TGCCAAGTGACATTCTTCTCACTGTGGTTGTTTCAATTCTTTTTCCTACCTTTTACCA
GAGAGTTAGTTCAGAGAAATGGTCAGAGACTCAAGGGTGGAAAGAGGTACCAAAG
GCTTTGGCCACCAGTAGCTGGCTATTCAGACAGCAGGGAGTAGACTTGCTGGCTAG
CATGTGGAGGAGCCAAAGCTCAATAAGAAGGGGCCTAGAATGAAACCCTTGGTGCT
GATCCTGCCTCTGCCATTTCTACTTAAGCAAGTTTAAGGCCTTCCACAAGTTACTTAT
CCCATATGGTGGGTCTATGGAAAGGTGTTTCCCAGTCCTCTTTACACCACCGGATCA
GTGGTCTTTCAACAGATCCTAAAGGGATGGTGAGAGGGAAACTGGAGAAAAGTATC
AGATTTAGAGGCCACTGAAGAACCCATATTAAAATGCCTTTAAGTATGGGCTCTTCA
TTCATATACTAAATATGAACTATGTGCCAGGCATTATTTCATATGACAGAATACAAA
CAAATAAGATAGTGATGCTTGATAGTGGTGCTTCCCTCAGGATGCTTGTGGTCTAAT
GGGAGACAGAACAGCAAAGGGATGATTAGAAGTTGGTTGCTGTGAGTTTGTTGCTA
TGGAAGGGTCCTACTCAGAGCAGGCACCCCAGTTAATCTCATTCACCCCACATTTCA
CATTTGAACATCATCCCATAGCCCAGAGCATCCCTCCACTGCAAAGGATTTATTCAA
CATTTAAACAATCCTTTTTACTTTCATTTTCCTTCAGGCAAAGAATCTAGATGCAATA
ACCACCCCTGACCCAACCACAAATGCCAGCCTGCTGACGAAGCTGCAGGCACAGAA
CCAGTGGCTGCAGGACATGACAACTCATCTCATTCTGCGCAGCTTTAAGGAGTTCTG
CAGTCCAGCCTGAGGGCTCTTCGGCAAATGTAGTGCGTTATGCCTAAGCATATCAGT
TTGTGGACATTCCTCACTGTGGTCAGAAAATATATCCTGTTGTCAGGTATCTGACTT
ATGTTGTTCTCTACGAAGAACTGACAATATGAATGTTGGGACACTATTTTAATTATT
TTTAATTTATTGATAATTTAAATAAGTAAACTTTAAGTTAATTTATGATTGATATTTA
TTATTTTTATGAAGTGTCACTTGAAATGTTATATGTTATAGTTTTGAAATGATAACCT
AAAAATCTATTTGATATAAATATTCTGTTACCTAGCCAGATGGTTTCTTGGAATGTA
TAAGTTTACCTCAATGAATTGCTAATTTAAATATGTTTTTAAAGAAATCTTTGTGATG
TATTTTTATAATGTTTAGACTGTCTTCAAACAAATAAATTATATTATATTTAAAAACC
AGTGACTGAAAGACGCATCTCAGCTGGTAAAGTTCTTACCCAACATGAGCAAGGTC
CTAAGTTACATCCAAACATCCTCCCCCAAATCAATAATTAAGCACTTTTTATGACAT
GTAAAGTTAAATAAGAAGTGAAAGCTGCAGATGGTGAGTGAGAGATGCCATGAGA
AAGCATTGCATATACCACATTAGTTAATTTCAGGTCTTGTACATTCTTTTCTGGACAT
GAGAGAGTAAGGGATCTAACTAAGCCACCTTTTGGAAACATAAAACATAATCTCTG
ATTTGAATTCAAGTCTACCTCCCTCTAGGTCCATTTTTAACTTTTAGTTGTAATTTGA
AGACAGATATAGAAAAATCTCAAAACATTTTAATATGAATTATACACTTAGAGTTG
ATGTCACAGATTCTGAGACCATGGGACTACTTAGATAAGATATAGCTCCAAAAGAT
AAAAGCGCCAAAATAATATCCAGAAGTTCTGCCTCCCTCGTCTGGAGTCTCCATGCA
CTGCATACCTCCTATTAGTGTCTGCCATTATATATCATACCTTAAAACTGAAGGAGC
TTTCTATCCAACTAGCATATGGGTCCCTCAAGAAAGCAGACTCTAGTGTTTTAACCT
TTTCGTGCTATATATAGGTAAGGAGCCTGAACAAAGGAGACCCCTATAAGTATTTGC
TGAATGAAAAGAGAATAGTTAATCACAGTATAACAAAAGTCAGTTCTTGGTAAATA
CAGAGCATTTGGGTGACATTACAGTGATGTGTTATTGTCTTTTAAAAAAAGTAGAAA
AGAATGGAAATGAAACATTTTAAGGATTTCTAAATAAGGGGCAGATACAAGAGTAT
TTTGGGTTTTAGCCCAGACTATACTGTAGGGGGAAAGCCTGTCTCAACTTTATCCCA
ATTTCATATATGCTATAACTTAATGTGGTTCTTCCTATTTCTGTACAAAACTGAGAAT
TTGGTGCCAATTTTATTA
SEQ ID NO: 3, Tslp^{em3(TSLP)Akp}
TTTCTAGAAGGAGAAAGAGGAGGGAGAAGTAAACAAAGCACAAAGAATGAGAACT
ATCATTAATATAAGAAATAAAAATTAAGAAAGCAAGTGAATGTTTTTCTAGTGAAA
GTGGGAAAAAGGATGGTTACAGCATGGGTCATCTTCTGGTCTCCCTGGGTAAGAAA
ATTACCAAACTCCCTGAGTAGTCACACAGCTCCAATGACATCACTTCTATTTCCTAC
CAAAGAGAAGGTGTCCCAGTCTTAATCCAACCTAGGATTTCCCAAACTGCACATGT
AGATACTGTTCATTCCTTCAGCATTAAGTATTTGGATTAAGATAAAACCAGGAAGCT
CTTCAGCCCACAGGAATTTCCAAAAATATACCTTGGCCCAGTGGTTGCTCCAGGTAA
GCCTAAGTAGATTCCAAGAAGGTGGCAGCAGTGAGCTACCAAAAGAAATCTCCGTA
GCAAGCTTGTTTCAGTGGGAGACATCCCTGCCGTGGCTTTCCGGATATCAGTAGATC
TGAGGAAACTCAGTTTCCCCTTCTCGTGTGAATAAGCTGCAGACCTTGCTGTCGTCT
GCACTGCCTTTCAGTGGTTTGAAACCTGAATTACTCCGTTGTCTCAGTTGTCTTTTTC
CCCAGTTCTAATAATGATTTCTCTATGTCCTCCCTGTACCTGCTCACACTTCCTTGTC
CCTTGATTCCGTTCTTATCTTCAGTAGGTTTTGTTTGCCTGATTGCTTCCTTGTTTTGT
ATTTTTTTTGGGGGGGGGCACTTCGACGTTATTATATTCACATAAATGCTTCAGAGC
AGAGTTAATGATTACTGGACAAATCAGTTATTACAGAACATGCCGGGGGGGGGGGA
TTGAAGAGGGGGGGGGGGGAGAGAAGGAGGGATGGATGGAGAGAGAGAGAGAGA
GAGAGAGAGAGAAATATGATGTAATTAAACATCATTAATGAAAACCCCACTGTACA
AATAGGACGAGCACTGCGCAACTCAAATCAACACCTAAGAAAGTGAGAGTGTGGA
AGGAGTCAAAGGAAAATATGAATAGCTACACAGGCTGATCCCTTGAGGGTATGTGA
CATCTCTCCTGCAGTTCCCCAACCCTGGAATATGCATGACACTCCACTGCAGCTCTC
TTAGAGACTCTCCCTTCTCCTCCCTTCACATTTAGAATCCCACCCTGGATTTAGTGTA
ACCAGTGACTTAAGAAGGTACCGCATATGGGAGACAAAGATACAAAAATCCTGAA
AGGGTTCTGGATTATTGGGCTCAGGACTCAATTCATCCGTGTTATCACAATTAAAAG
TAGTCTTTCCTTAAAAAAAGCCTTGGTTTCTGCATCTCTGTGATCAAAATCCCATAA
CAAGGTTTGGAAGAGGCAAGTTTGGGAAAATTTCAGAGTGTATTAACTTAGAATAC
TGTTGGAGGGAAGCCTGGGTAAATAAAGGAGATAAGGTTAGAAAGAAGACTTGAA
GTCAACATGGGAGTGATGAGTGAGAATCTTAAAGTAACTGAGTCTACCAAAAGTCA
ATATAATTGAAATGACTTAAGATGTCACATCATTACCAGTAAGGTAGCTGGATGCTA
TGGTGTAGGTGATGTGCTTAGCAAAGAGATGCCTTCTAAAAATCCCTGAAGGGGGC
CCCATGCCTGCCTCAGATTTACCTACACATACATAAACTATAGACACACTTTAAAGG
AGAAACCAAAAATGGCAGGTAGGCTGGGTGCACCCCAATGGGTGCCAAGCCAAAA
CTTATGGGGGTCAGGGGACAGGTTGTCTGTTGCTGTCTGACATTCTTGCCCCCATCA
GCAATTATTCCTGGGCACTGCAACACATGAATCTACCCAAAAGATTCGGGCGGAGA
GGCAATATACATGAAGTGACTTTAAAGACCACGTGTTTACCAATAAAGAAGTGGGT
TCCCTACAGGGGAAGGCAAGTGAATGAAGATGGCAAAATCAGCTGCCATTTCCTTT
CTTTTGTCTCTTGGAACTATCCCAATTCAGTGACCACATCTGGATCTCTACATTGCTT
CTGCCTATGCAATATCTAGCTGCTGATCAGAATCATATCTGATGTCACGCCAGATGA
ATCAGGCTTTGGCATCTTCCCTTATCACTGTAAGAAGTAGAGATGGGAAGACGCCAT
GATCCAGACATGGTATCATAACCTAAATTTAAATCTTGCAGGACTCCAGAAAAGTC
CGTCTCTAAAGTCATCAGCAAAGCAGAAACTTTCTGAGCCTCCTGCCACCGCTACAA
TCTTTTATTCCTCATCCTAATGCCAGAGAACTGGGTCCAGCTGTGCTGCTCCAGCTGT
TGAAGGCCTTCTGGGAAAACTTCACCTCTGACTCCAGTCTGTGCTTTCCCCCGAATA
GAATCATTTACCAATCCCTGTGCTCGCTCCTTCCCTGGCTCAGCGTGGTCTGTGACAT
TTTCAGGGACTCACGTGGAGCACCCAACATCATCGTTCTGAGCAGTGACTCCTAGGA
ACTTCCCGAAGACGAGACTGATGCAGGCTCTGACACGCAAAAGTGGGGAGAGTGA
ACTGGGTCTCAGGAGGGCCTGGGGCAGCTGGCTGAGCTCCAGGAGAGTAGGGGTTG
GGTTCGTGTCAACAGCTGGGCCTTTCTTTCCTGCTCCCAGTACTGTACTGGCGCTGCT
CCAATCAGAAGGCTGCGAGACATCCTCTCAGGCTATCCCTGACTCACTTGGCTACTT
TTATCTTGTACTTCCTTTCAAACCCCAACCAGGGGAGCGCAAATCTTAACCCAACCC
ACCATCCAGCTTCTTTCTCCATCCCTGACAATCGTGCTGCTGGGACGCATGCCTGGG
GCCATCCAACGATTTACTGGCTGAGAGTCTGAGCTGACACAGCTCAACAGGTCAGA
AGCTGTTCCTCCCCTAGGAGGAGAGCATGGTGGACAGGTCTCTCTCTAGTGGCTTAG
ACCTGCAACAGCACCATAGCACCATACACCTTAGGAGCCCCCACTACTCCTGGTAA
GGCATCTTTACTCCACTGAGACCTAAATAATGAGTTTCGAGGGCGGCTGGATGCTTG
ACTTCATCATTTTAAAAATCTTAGTCACTCTGTAGACCAGGCTGGCCTTGAACTCAG
AAATCCATCTGCCTCTGAGTCCCAAGAGCTGGGATTAAAGGCGTGCGCCACCACCG
CCCAGCTACCAGTTTTCTTTAATCAAGCTTAGGCACTCACCCTGATTCTGAGTTTTTG
AAGATGAGACTAACTGGTCCTTTTCTCATATATTTCAATTTCTCATTGTTCCTGTTTC
CAGTATTCTGACAACAACTGCCCGGTTCCAGTGAAATGCCTTCAACAAAAGTTACGT
TATCCCAAGGCTGCATTCATTCTCCAAAATCTGTCATACAGGAACACTGCGTTTCTC
GGTAGCCACGAAGAGGAACACTGCCAGTTCAAACTGGACAAAGGAGATAGATGGT
CAGGGTGTGCATGGTGGAACAGCATCAGTAGCAAACCCCTAAAGTGACTGCGGGTG
TTAGAAGGTGTTTTTCCAAGCAGAAAAAAAATCAGTCATAGAAACTGCCCAGTAGG
AAAAAGATGTCAAAATGATGACATGGTATCATCTCTAAAAGCATATCGAAGCATGT
AGCAAGTGTTTAGGGCAGAGCTAAAAAATAAATAAATAAATAAAAATAAAATAAA
ATAAAAGGAAAGGAAAAAGGTGAGGGAAATTCCTGATGATTTTGCTAAAGTTAAAA
TTCCATAGATTTGGCTGGCTTTATTTCTTTTTTTTTTTTTTTTTTTTTTTTACATCATCA
ATTTAGAATTCTATAAAGAAAGAATGACATCAAGGAAAATCATTGGCCTAGGGGAA
GAGAGCCCGTAGGCGTTTAGGTGTTATAAATATGGAGGCAGAGAACACTGGAGGAT
CAGGAAGACTCGCAGCCAGAAAGCTCTGGAGCATCAGGGAGACTCCAACTTAAGGC
AACAGCATGGGTGAATAAGGGCTTCCTGTGGACTGGCAATGAGAGGCAAAACCTGG
TGCTTGAGCACTGGCCCCTAAGGCAGGCCTTACAGATCTCTTACACTCGTGGTGGGA
AGAGTTTAGTGTGAAACTGGGGTGGAATTGGGTGTCCACGTATGTTCCCTTTTGCCT
TACTATATGTTCTGTCAGTTTCTTTCAGGAAAATCTTCATCTTACAACTTGTAGGGCT
GGTGTTAACTTACGACTTCACTAACTGTGACTTTGAGAAGATTAAAGCAGCCTATCT
CAGTACTATTTCTAAAGACCTGATTACATATATGAGTGGGGTAAGTGAAGAAGCTTT
TTTAAAACAAATGTATTTTCATCAGAGGAGTCGGCATACACACACTCTACAATTTAA
CTTTGTAGGAAAGAAAAATAATTTAGAAAAAATCATGGCCCCACATTTTGTCAAGG
ATTCTTACAAGTGATATTCAAATATCTAATCTAAAATGATTATCTAGAAATTGGCAC
ATTCTAAGTGTGCAGATGCTGATGAGGAGCAGGTATTGATAGACAGCGCGTTATGC
GTCAAAGGATGTCTATCCTTTGCTAAAGTGTTACTCTGACTATGCTGTAAAAAGCAG
GAGGTAAGAGCTTAAGAAAGAGGAGTAAAAGAGATAATTCTCATGAGATAAACTCT
AAGGATTGATGCTGTGCTCCAGGTCTCTCCAGTGTTTTAGATGTTTCAGGATGCTAT
TTATTACAGAATATGGTGTACTTGGAAAACATACAGTAGTAATCATTTTCCTGATTA
ACCTAATTTCTAGACAGAGTTTGCATTCATGAATGGCCACAGTACAGATGCGGACAT
CCAAAGGATGGCATTATTACTCACAAGCATAGTGCTATGTGCAGTTATGGCTTGAGG
GAAGGGAGGGGGGAGGTCGCCCTCTGAGACCTGAACCTTTTGGTGTGGTTTCAAGC
ACTAACCAGCACTATCTAATGGCTATTTCACTGCCTTGTCAATGACATAGGAAAAAG
GTACCTGAGTGGAAACTGTTTTCAGGGCACCTTTAAAGCCTGGGAGCAAAGGGTGG
AGGGATGATTTTCCTTGTGGACTTAAAAGTCTTTACCCTCTTTGTCCTATTTTTCTTTC
TTCCAGACCAAAAGTACCGAGTTCAACAACACCGTCTCTTGTAGCAATCGGGTGAG
TAGAGAGTTCAGTGCTGCTGGCTTTCTCCAGGGAGACGCCAGGCATTTTGGAGAGG
GAGTATCCTGCTACGTGCAGAACTCCGAGAGGTGCCTGGGCTCCGGGACGCCGCCG
CCGGGGGAAAGGGGACATCTGGGCTGTCAGAGCGGGGCTGCGCCTAGCTTGGGACA
ACACTTCTGTTCCAATTTAGGGAGAGGAAGTCTCTATCCGGAGGAAAGGCAAATTG
GGAACTGGGACGAGGGAACGTTGTTAGGGGCACCACCTGCTGGGGTCCGGCGCCTC
CGCGCTCGGGCTCGGAATTTTGGCAGCCTCCGCCCCCTGGAGACTTGGGAGGAGCG
AGCGTGGGTGACAGTCTTTTCGCGACGAGTGCCCTCCGCCACCCTCGCCACGCCCCT
GCTCCCCCGCGGTTGGTTCTTCCTTGCTCTACTCAACCCTGACCTCTTCTCTCTGACT
CTCGACTTGTGTTCCCCGCTCCTCCCTGACCTTCCTCCCCTCCCCTTTCACTCAATTCT
CACCAACTCTTTCTCTCTCTGGTGTTTTCTCCTTTTCTCGTAAACTTTGCCGCCTATGA
GCAGCCACATTGCCTTACTGAAATCCAGAGCCTAACCTTCAATCCCACCGCCGGCTG
CGCGTCGCTCGCCAAAGAAATGTTCGCCATGAAAACTAAGGCTGCCTTAGCTATCTG
GTGCCCAGGCTATTCGGAAACTCAGGTAAGCCCGAAGCCTCAGACGTTTGCTGTAC
CTTGGGGCTAACCTCAAATTAAACTGGGGCTTTGGTGCAGAAGTCGTTCTCTTATTT
TTATTTAGGTTTTATCTTTCGAAGAGCAAACGAGCCGGGTAAAAGTGGTAGGATGTC
AGTTAGACCCACGTTGATACCCGGAATCAAACTCACCTATTTCTACGGTTCTGATAC
TGTTTTGGCTGAATTATGGTTCTAAACCTTAGGGCAATGTTTCAAGCTATGATGAGT
GAGACTTCTATATCAGAATGTTTTGATTGCTGGAGCATAAGAGTATGGCCTCTTGTT
CTTATCACTTAATTATTGTGTGCTTATTTGCTAAATGTATAATTACATTATACATAAA
ATCTCTATCCTATGTTTGCTTAATTGCTTGTGTGGGCGCTATTGCTGTCTCTTTACAC
ATTTTTGCACATGTAGTTATCTGCATTTGAATGCTCGTGTAGCATTAAATATGGAGTT
TATTTCAGTCAGCAAGTAGAGGATTTATCTTCATGGTGACAAGTTTAAGGAACAGA
GAGAGACAAGTGCAGATATGTTTGATTGCTCCTTATTAGCCTAGTGGACTTTATATG
TCTACAGTCTAGGTAGATGGACACGACTGTCACAAAACTGTCACTTTCTAGAGGTTG
AGGATTGAAGCCATAGCGCTGATCTGGGTTGAGCTTGAATTAGAAACTCAATACCA
GACAGCCATATGGGAAACCTATTTGGCTTCATGCCTTCTTATGAAGGAGACCCTGGC
AAATCTGCAGATGGCTACAATAAAATTCATTTAAATAAGAGCACAAACAAAAAGCT
AGATCAAGTTCTTGGACAGCATGTGAGAAAGGGAGAGTTTGGAGAAATTTATTTCA
GTCCCTCCCAAGCCCAAATGGAGAGTCTAAGACTAATAATAATGATTTTGCAGGTTT
TTTTAAGATTTGTGCTTAATAACCCTGTGACTTTATTAATTTGCATACCATGTGTCTA
GGAGGCCCAGTGTACTACTCAAAGGTAATTCAGATAAAGGTATATACTGCAATCCT
CTTTAAAATAAGCCCTCAGATGTCTGTGACACATCTAGACAATGGGGCAGGGGAGG
GGGAAGGATGGGGAGCAGGAGCATGCATTTTGGGTCCAAAAAATAGACTAGGTTTA
TTGAATGATGTCTATAAACAGGTATAAGATAGCTCTTGCCCATGAGGAACTTGTGAT
CTTGTCAGGGAGGTCTTGAAATCAGCAATTTATTCATTTCATGTTAAGTGAGAGCCA
AGTTAAATGACACACACTCTTAAGTACTGGAAGAGTTTCCAAAAGCACCTGGAAAA
GGCACATGCTAGCACATAGTAAGCAGGTGCTTTGGAGACACACTGAAAGATGGATT
TGCATAGAGAAGGCAATTAAACCTGCTCTCAACAGTTACTAAAGATAGTGAAAAGT
AATTTTGACTATTGATTCTTATATTCTGCAGATAAATGCTACTCAGGCAATGAAGAA
GAGGAGAAAAAGGAAAGTCACAACCAATAAATGTCTGGAACAAGTGTCACAATTA
CAAGGATTGTGGCGTCGCTTCAATCGACCTTTACTGAAACAACAGTAAAATTAGCTT
TCAGCTTCTGCTATGAAAATCTCTATCTTGGTTTTAGTGGACAGAATACTAAGGGTG
TGACACTTAGAGGACCACTGGTGTTTATTCTTTAATTACAGAAGGGATTCTTAACTT
ATTTTTTGGCATATCGCTTTTTTCAGTATAGGTGCTTTAAATGGGAAATGAGCAATA
GACCGTTAATGGAAATATCTGTACTGTTAATGACCAGCTTCTGAGAAGTCTTTCTCA
CCTCCCCTGCACACACCTTACTCTAGGGCAAACCTAACTGTAGTAGGAAGAGAATT
GAAAGTAGAAAAAAAAAATTAAAACCAATGACAGCATCTAAACCCTGTTTAAAAG
GCAAGGATTTTTCTACCTGTAATGATTCTTCTAACATTCCTATGCTAAGATTTTACCA
AAGAAGAAAATGACAGTTCGGGCAGTCACTGCCATGATGAGGTGGTCTGAAAGAA
GATTGTGGAATCTGGGAGAAACTGCTGAGATCATATTGCAAATCCAGCTGTCAAAG
GGTTCAGACCCAGGACAGTACAATTCGTGAGCAGATCTCAAGAGCCTTGCACATCT
ACGAGATATATATTTAAAGTTGTAGATAATGAATTTCTAATTTATTTTGTGAGCACT
TTTGGAAATATACATGCTACTTTGTAATGAATACATTTCTGAATAAAGTAATTCTCA
AGTTTGTTTCATTCATTTATTTATTTAGTTAGTTAGTTAGTTTGGTTTTTTGAGACAG
GGTTTCTCTGTGTAGCCCTGGCTATCCTGGAGCTCACTCTGTAGACCAGGCTGGCCT
CGAACTCAGAAATCTGCCTTCCTCTGCCTCCCGAGTGCTGGGATTAAAGGCGTGCGC
CACCACACCTGGCTTTCAAGTTCGTTTCTTATGAATGGCGTTTTAAATTTGGTTGAGC
AATTTTCATGCGTACTTTTCTAAGGGACATCACGGTTGTCTACATCTTTATCGCCACT
CAAGCCGACATCCCATGGGCCACACTTCCTTTGATCTGGTATCAACCCTCCCTGCAG
GAGAAAAGGTCTTCATAAGTAGTTGCCTCTTGGACAAATGACTGGAGTGCATTTTTT
TCAAATATTTGCACCAGTCACTCCCTCCCACTGTGAATCTTTCTTCACCTCAGAATAG
ATAACACAGGTGAAAATGAACAGTGGGTGTTAAATTCATTCCTGCACACCTCTGGT
AAAACACCCTACCTCTTGCCCTCAGAATCTTCTGAGCATTGCTAGCAAAGGCAACCT
TGGCTGCAGAGCTCAGGCCAAGTAAGAGTAGATGTAAACAGCTAACCTGCTCCTCC
ACCCTACACACACTCTAAGAAGAGATGTTCACTTGAATACTGTTTTGAAGGTTAGAA
CTAACCCATTAATGAAAAGAAAAGCTGAGTGTCCCCAAACCTGTCTTACTTGTTGGG
AGCGACCCTGTTGGAATGTTAACTGCCTTGTCAGCCATAAGTGCTTACTTACAAAGT
CTTGACCTTAGTGGAAAAATACTAGCTTAGTTGAGATTTCTGTGGGAAAAGTTGAAG
CCTTTGTAGGAAAGTACTACCCCCAGTTAAGAACAAATAGTTGTGCTCACTTTGGCA
GCACATATACTAAAATTGGAACGATACAGAGAAGATTAGCATGGCCCCTGCGCAAG
GATGACACGCAAATTCGTGAAGTGTTCCATATTTTTTGAAGCTGGGACGAAAGGAC
GGACCATCTAGTGATTGCCATATCCAGGGATCCATCCCATAATCAGCTTCCAAACGC
TTGACACACTAGCAAGATTTTGCTGAAAGGACCCAGATATAGCTGTCTCCTGTGAGA
CTATGCCGGGGCCTAGCAAACACATAAGTGGATGCTCACAGTCAGCTATTGGATGG
ATCACAGGGCCCCCAATGGAGGAGCTAGAGAAAGTACCCAAGGAACTAAAGGGAA
CTGCAACCCTATAGGTGGAACAACAATATGAACTAACCAGTACCCGGGAGCTCTTG
TCTCTAGCTGCATATGTATCAAAAGTTGGCCTAGTCGGCCATCACTGGAAAGAGAG
GCCCATTGGACTTGCAAACTTTATATGCCCCAGTACAGGGGAACACCAGAGCCAAA
AAGGGGGAGTGGGTGGGTAGGGGAGTCGGGGGATGGGTATGGGGGACTTTTGGGA
TAGCATTGGAAATGTAAACGAGGAAAATACCTAATAAATTTTTTTTTTAAAAAGTAA
AAAAAAAAAAAAAAAAAAGAACAAATAGCTATAGATCTTGTGGACAGGTACCTAG
CAACCCATTCTGTTCTGTTCCTCCTGCTGAACTTTTTACCTAGCCAGTATCCTGCTTT
TGGAACAGGTGCATTCCCCCAGAAACAAAGCGATTCTGCATCGTCCCCCTCCACATA
TCCTGCTTCTGTGGGTATAAAACCTGCCTGGGAAAAATAAAATTTGTTAGTTTGATC
AGAATCTTTGATTTGCTGTTCGTTTTTTGTGTTTCTTGCCCCGCCCCCCTTCTCTCTGC
AGGTGGTTCCTCAGACCCTGTTCAACTGTCCCGCATCAGGGCATTACTTGTCAACAA
AGAGCTACTTATGAGCACCAAGTAAATAGTTACAAAGTGCCCACTGTGGGCCAACT
TTCCTGAGGTGAAGTCTGTGTTAAACCCATAGTTACAAAAGTAAGTAAGACAGAGC
TCATATCCAGAGAAGCTCAGAGTGGAACTGGATAATCAGTTGTCTGTAGTCCTTAAC
AAATTGGCCAGTGAGTGTTCCTTTGATTTGAGTAAAATCAAGACAGGCACACTTTCA
AAAATCTTCCTCTAAATTCCTTACCCAGAGCTTTTAAGCACCACCCTAAGAAAACTC
CACTGGGTCTAGAAAAGGCAGCAATCATCAATTCTTTGAATAGAAGTGTGGAGGCC
TGATATTTTAAATGTATTAACTCTGCCTTACTACAAATTCATTCTCCCTTTTACTAAA
TCATGATAAAAGGTATTATAGCATTTTTCTTAATCCCTTTAGACCCAATTGCCCTAA
AAGTGACTTCTACCCATTTGGTAGAGTTCATAGGACAGAGTACCAAAGGAAAGGAG
TGCTCTGAGGAGGAGACCATTAGAAGATAACTCCTGTTATTGAGGACAGCAATACC
AAGCACATGCCTTAAGAAAACTGCACTGGAGAGATGGGGAAACATCTGGACAACA
AGAGGGACTAGTGTCCATTGCTCACTGCAAGCCAGGGAAATGAGCTGTGCTCACCA
GGCAGAATGGAAAATTCTGTAACCCATCAGGCTATAAGTAGGGTACTGTGCTGACT
AGTTTAATGGCAACTTGACACAAACTAGAAACATCAGAGAGGAGGAAACCTCAGCT
GAGAAAATGCCTTTATAATATTCAGGCATATGGCATTTTCTTAATTAGTGATCAATA
TGGGAAGGGTCAACCCATTATGGGTGGGGTCATCCCTGGGCTGGTTCTAAAAAAGC
AGGCTGAGAAAGCCATGGGAAGCAAGCAGCTTCCCATCATGGCCTCATATTAGCTC
CTGCCTTCAGGTTCCTGCCCTGCTTGAGTTTCTGTCCTTACTTTCTTTGATGATGAAG
AGTGATGTGAAAATGTAGGCCAAATAAACCCTTTCATCCTCAACTTGCTTTTTTGTC
ATGGTATTTCATCCCAAATATAGAAACCCCAAGACATGTGCTTAAAAACATCTTACC
TGTGCATGGAAGTATCGTTAGACCAAGGCTAATGGCTGCAACGATCTAACTTAATG
AATTTAAAAAAAAATAATACTTAAAAGAATCGGTTCCTAAGTAACTTAGCTGTATTT
CACAACAAACCACAAGGGTGTTTATGAAGTAAAGAATGTCTCACACACATGCGAAT
GTATCCACTCAAATATATATATATATAATTAAAATAAATCTTTAAAGAATGAAAGA
AAAAAGAAAACAAAAGGGGAGAGAGGGGGAAGGAGTAAGAGAGGATCTTGAGGA
CAGAAGAGCTGTAAGAACTATTGTGTCCTGTTAGGGAAGGTGGCACACCTTTTATCT
AGAGTCAGAAAGCAGGCAAATCTTTATGAAGACGAACTTCATCTATACAGTTTCAG
GCCAGCCAAGCTATACAGTGAGAGCATGTCTCAAAAATAAGGAGGAAAATATGGTG
CTGTAGTATAAAAAGTACCACTAACTCAAAACTAACATAGAAGGTAGAATTAATAA
GTGAAACATTAAATTAATTATTATAATGTTGAGAACAAGCAAAAGAAACTTATCCT
AAGTTAACCATTCCCTTTTCAGACTCCTTTTAATTGTAGTGAGAAACTAAAATCAAA
ATCCCAGGCCCTAGGGGAGCTTGGAAATTCCTAACAGCTGAACAGTTTCTATTTTAA
GGAAACAGTTGTCCAAGTCCAGATAGCTCAGGGACAACTTCTCCATCTTGCTAGTAA
GATCAAACTAAGTTCAGGTTTCCAGGCCCAGAAACCTACTTCTATCCTTATTGATAG
AAACTCCCTTGTTCAACTTCTTATGTCAACATATGATTGGACAATGTTATAGTCTACC
CTGCTCCCCCTCACTTCACAGTTTTGATTCCATTCTTTAAATAGGCTGTACAGTGTCC
CTTCAGAGTTGCAGCTCAGCACCCAAGTCTGTTCTTTGGCCCTAACTAGTAGACACT
TAATTACAAGAAAATTTTGCCATCTGCATGGTGTTTGAATTATGTTGTATTTAAGCA
GACCCCACAACAATAACTCAAGATATTTAGGAAACATAGAAGATACAAGCACAGAT
TCTAGATATGAAAATTATGTGTAAAATAAATACACAGTGAATAGTTTTAATTGGGG
GTTGGGCATTAAAATATTTGAACTAGACCAATACCCACCCAAATGCTACAGCCTGG
ATGCTCCCCAGGAGATCCAAATTGACCAAGGACACAAGTGACAATTTCACCCAAAT
ACCCATGCCAGCTGGAACACCCACACAGCCCAGCTGACATGGGACCTACACCCCCA
ACTCTCCACCCTCTATCCCACCCCTCTGAGATCCACCTTCCTTCTGATCCAATTCCTC
ATCCAGACCAGGTCAGAGACCTAGCTGATACCTGCCCAAACTCTGCAGCCTGATCTT
CCCAGGAGGTCAGCAGTAACCAAGGGCACAGGAGGTCCACACTAACCAAAGACAA
CA
SEQ ID NO: 4, Ltbr^{em1(LTBR)Akp}
GTGAAATGTATCTAGGGCCGCTCCCCACCCACCCGTTCCTTTATGCTGTTAAGAGAT
CCAAGTGAGTCAAGCCCCTGCCCCAACTCCCTGAGCCCAGAAGGAAGAGAAATCAG
AGGTCTGCTATTCAGTATCTCTACCACTGCCAGGGAACCTGGGACAATTGAGACAG
ACAGGTACCAGCAGAGTGGAGCTGCTGGGCCAGCACCCAGGGAGGGGACAGCACA
GAGTGACTATCAAGAGCCCAGGCAGCAGTTAAGAGCATCAACTGCTCTTCTGAAGG
TCCTGAGTTCAATTCCCAGCAACCACGTGGTGGCTCACAACCACCACTAAGGAGAT
CTGACTCCCTCTTCTGGAGTGTCTGATGATAGCTACAGTGTACTTACATATAATAAT
AAATAAATCTTTTTTTTAAAAAAAGTATACACTTAAAAAAAAAAAGCCCAGGCAAG
TTCCCCCCCACCCCGCCCCGCTCTGTTCCCCCCTCTCCCCAGGTCTTCTCTAAAACTC
AATCCCTTGCCAGCATTTCGAGGCTCCACCGAAAGCCTGTCTGGATATCTGATCCAC
CATGGAAAAGGTCAGTTCTCAGGTGAGCTATTGCAAGAGAAGGCTTTCCCTCATTCC
AAATGAGAGTCCAACCCCACCCCCCACCCCCGAGTCACAAGGGAGGCAGGACAGAT
GTTGCCATGGGCTGGGATCTGAAAATCAGATCTGGACTGCAGTTAAGTTCTTCCAGA
GTGGCTAAGCGGTGTGGACAGCCTTCATTTACACAACGAATATGTACCTGGCCAGT
GGCATAAGCCAGATCATGCTAGACTCCGTGAGCCTAGACTAAATAGCCAAACCAGG
CCACGGGCCAGCCAATAGCCTAGTAAGGAGCACAGGGCAGACATTAGGGTTCCTTG
GGGGCTCCATGGCTGTTTTCTTAATAGAAATTTAGAGGGGTGTGTGTGTGAAGGGG
GCTGGGGGGGGGTTGGGAATCAGTAGACGTGGGAAAAAAGGGTGTCACATACTTCC
AGCAGCTCTGGGCTATTAATGGCAGGAAGAAAAGGCCCACCAGGCTTAACGATCTT
GGAACCCTGTGCACCGCTGCCTGGCACCCTGGGCAGGTCTTCTCTAGAAAGTAAGG
TACCTACACTGGCTGGGCTCTCAGGTCCCTCGGTTTAAGAGAGTTATAGGCCTATGT
GCACACACGCTGGAACTAGGCTACCCAGCCCCAGCCCAGAAGCCCCCACTCACCAG
GACCTGGGTTTACACACGCCCACCCTTCCGTGAGAGAGGTCCCAGAGGAGAGGACA
GATTCAGGGCCAGCTGAACTCTCTCCAGTGTCTTGTGGTGTGCGCCCTGGTGTGTGG
CGTGGGTGGGCTTGTTACTGTGGAAGCTTCTTTTTAAAAAGTCACAGAGTGGAGCAG
GCCCTCAGTCTCTGCCAAGTGGGATGCCTGGCCAGACGCTGGCTGCATCTGCTAACC
ACCTCTGGGTATCCTGGCTGGGTGCACTGTCAATCCCTGGCGCCTCCTCTTTGCAAA
TCTGACACCCAGCTGTCCACAGCTCTCTGCCTCAACGTCCACGGCAGGTCAACCAAG
TCAGCTCTGCCTCGGGCTCTCGGAGGTGGGCCTGACTGATGGCTAGCCACTGTCTCT
GCTGCCCCCCTTTCGGCCAGCAAGCGATCCTAATCCGCAATCCCCTCTGAGAGCCAG
GCTTCCGAAGAAAGGTGGAGGCCGGGTTCCGGGCCTGCAGCTCTCACGTGCTTTCCC
GGCCACCCCCTCCCGCCCTGCGTCGAGGCGGCCAAGCCTGTTCCTCTTCCCCCCCGT
CGCGATTGCGACAGGCCGGCCTCTGCTCCCAGGGCTCCCTGCCCCCGCCCCCGGCCG
GCTCGCTCCACTCCCACTTCCTGAGCCCGGCGCTGGAGCCCTGGAGGCCAGGCCCG
GCCGCTCCCGGCCCCCGGGGGCACGTCGGCCCAGCCGCCAGGCTTGGGAAGTCGTG
GCCAACGCTGCTCAGGACGTCCGGGCTTCCCACCTTCCTCCTAGGACTCACCCGTCT
GGTCAGCCGAGCCGAAAGGCCGCCATGCTCCTGCCTTGGGCCACCTCTGCCCCCGG
CCTGGCCTGGGGGCCTCTGGTGCTGGGCCTCTTCGGGCTCCTGGCAGCATCGCAGCC
CCAGGCGGTGAGGAAGGGGCCTGGTAGGAGTGGGCGAGGGTGGGCAAGAGGGATC
TGGGCAGCCGTCGCTCCATTCCCTCTGCCCTCCCAAGCTGACCCCTGACTAATTCTTC
TCTCCTCTTCTCCATCTCCCTTTGAAGGTGCCTCCATATGCGTCGGAGAACCAGACCT
GCAGGGACCAGGAAAAGGAATACTATGAGCCCCAGCACCGCATCTGCTGCTCCCGC
TGCCCGCCAGGCACCTATGTCTCAGCTAAATGTAGCCGCATCCGGGACACAGTTTGT
GCCACATGTGCCGAGAATTCCTACAACGAGCACTGGAACTACCTGACCATCTGCCA
GCTGTGCCGCCCCTGTGACCCAGTGATGGGCCTCGAGGAGATTGCCCCCTGCACAA
GCAAACGGAAGACCCAGTGCCGCTGCCAGCCGGGAATGTTCTGTGCTGCCTGGGCC
CTCGAGTGTACACACTGCGAGCTACTTTCTGACTGCCCGCCTGGCACTGAAGCCGAG
CTCAAAGATGAAGTTGGGAAGGGTAACAACCACTGCGTCCCCTGCAAGGCCGGGCA
CTTCCAGAATACCTCCTCCCCCAGCGCCCGCTGCCAGCCCCACACCAGGTGTGAGAA
CCAAGGTCTGGTGGAGGCAGCTCCAGGCACTGCCCAGTCCGACACAACCTGCAAAA
ATCCATTAGAGCCACTGCCCCCAGAGATGTCAGGAACCATGCTGATGCTGGCCGTTC
TGCTGCCACTGGCCTTCTTTCTGCTCCTTGCCACCGTCTTCTCCTGCATCTGGAAGAG
CCACCCTTCTCTCTGCAGGAAACTGGGATCGCTGCTCAAGAGGCGTCCGCAGGGAG
AGGGACCCAATCCTGTAGCTGGAAGCTGGGAGCCTCCGAAGGCCCATCCATACTTC
CCTGACTTGGTACAGCCACTGCTACCCATTTCTGGAGATGTTTCCCCAGTATCCACT
GGGCTCCCCGCAGCCCCAGTTTTGGAGGCAGGGGTGCCGCAACAGCAGAGTCCTCT
GGACCTGACCAGGGAGCCGCAGTTGGAACCCGGGGAGCAGAGCCAGGTGGCCCAC
GGTACCAATGGCATTCATGTCACCGGCGGGTCTATGACTATCACTGGCAACATCTAC
ATCTACAATGGACCAGTACTGGGGGGACCACCGGGTCCTGGAGACCTCCCAGCTAC
CCCCGAACCTCCATACCCCATTCCCGAAGAGGGGGACCCTGGCCCTCCCGGGCTCTC
TACACCCCACCAGGAAGATGGCAAGGCTTGGCACCTAGCGGAGACAGAGCACTGTG
GTGCCACACCCTCTAACAGGGGCCCAAGGAACCAATTTATCACCCATGACTGACTG
TGCCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGG
AGGATTGGGAAGACAATAGCAGGCATGCTGGGGACAGTGGCTCAAGTGGCTTGGGT
GGTAGAGGTGAGCCAGAATGAGCCAGCACTGCTAAAACTAGCCAGGAAGGAGAGT
CTACGAAGCATTAGCATTGTCCCACGGACACTGAGATTTGAAGAGGTAGCGGCATG
TAGCCATGAAGACAGGATGGGGACAAAGAGACCAAGGAGAGGCTCCGAGGCATGC
AGCAAGCAGAGGCAGCGGACGCAGAGATGGACTTCTTGTCTCCTGATAACCCTCTT
TCCCCATTCGCCTCATAGGCGAGTTTGTCTTTGCGGTATGCAGCCGCAGCCAAGACA
CGGTTTGCAAGACTTGCCCCCATAATTCCTATAATGAACACTGGAACCATCTCTCCA
CCTGCCAGCTGTGCCGCCCCTGTGACATTGGTAAGTGGGGACTCATCTGGATCTGCA
TGATGGGTACGACTGGGAGGGCCAGCTCCTCTCTGACTCTTCCCTCTCCCTGACAGT
GCTGGGCTTTGAGGAGGTTGCCCCTTGCACCAGCGATCGGAAAGCCGAGTGCCGCT
GTCAGCCGGGGATGTCCTGTGTGTATCTGGACAATGAGTGTGTGCACTGTGAGGAG
GAGCGGCTTGTACTCTGCCAGCCTGGCACAGAAGCCGAGGTCACAGGTCAGAGGTC
ACTGAGGGCAGCCAGTAAAGGGAGGCTGGGCATCAAGGGCAAGGAACGTGATACT
GTGCGCATGGTGCTTCTCCCCACTGGTACTGTGAGTGTGGTACCTCTGCCCACTGGG
AGAACCATAAAGAATCTATCAGTCCTTGAAAAAGGCTCACAGGAGGGGGTCTGCCA
AGACATGAACTGGTATGAGGAGCTTAGAAGGTAGCTCCCTCCTGTCAGCCCTGGGG
AAGCTTGGGCAAAACGGCAGGCTGCAAAGCCAAGCTTGGGAAAGGTAGCAACTAC
AGAGCAGAATGGTTGGCAAAGAGGGGACGTAAAGGAAGGCCACCGAGTCCTCACA
CTTACCCACTCACCCCCACTGGCCTGCCTTTCTTTTGCCAGATGAAATTATGGATACT
GACGTCAACTGTGTCCCCTGTAAGCCGGGACACTTCCAGAACACTTCCTCCCCTCGA
GCCCGCTGTCAACCCCATACCAGGTGAGAGGGCCCTTCCCCCACTCACCTCCAGGA
AACCCAAGGGTTGTCATCTCCTCCATCCTTGACTTCCGGCCATCCCGACCATGTGTT
CCTGGAGCCAGTCACCAAGGGGAGCAGGGAGAAGCTCACAGTCTTGTTTCTCCACA
GATGTGAGATCCAGGGCCTGGTGGAGGCAGCTCCAGGTACCTCCTACTCGGATACC
ATCTGTAAAAATCCCCCAGAGCCAGGTAAGACACCGGGCTGAGGAACACAAGGCA
GGGTCGGTCTGGGAAGATGCCTCAGCCCCCCTCATCCACAGAAACAGGGAACAGTG
CATCTTTCTTCCCAGGGTTAGACAAAGTCAGAAACATTTCTTCTGAAGAAATCAGAA
GGAGGTAGCGTGTAGTTCCATGGTTAGAACGCTTGCTTGGGATACATAAGACCCTG
AGTTTGGACCAAAAGAAAAAAAACGAAAACTTGGAAAGGCAGGTGTGGTGGTGCA
CCCTTGTAATCCCCAGCGCTTGAAAGGCTGCGGCAGAAGAATCAAGAGTTTGAGGC
TAGCCTTGGCTACAGAGTGAGCCTGTCTCCATAGAGGGCCTGGAGATTAGAACATC
CCTAGACTCTTTTCTTACACTTTCAAAATTATACATATTATGCCAGGAAACATTCCTG
TGCTGTGACGTAATTCTAACCGGCTTCATCACTATGCTTGGATGTGATTCCGTCATA
GCCTTCCTTCACTAATTGAATACCTCGTTGTTCACTTACACACATCTGTTGGAGACAT
GCTCCCCCACTGGGCTCTTTCTAGGTTTTCTTGTTTCTTGGTTTCTGTCTTCGAGGAA
ACCCACTAGTTTCCCAGCCTGGTGGTTGACTATAAGTTCTTCTGATGACTCTAATCG
CTACTAATTGGCAGAATGTAGTAACATTTTTGAGTGACCAGACTTTTGTAATTATAG
CTTCCACATCCTGAGAACAACTCTGAACCTCT
SEQ ID NO: 5, gRNA for mouse Flt3, 5′-AAGTGCAGCTCGCCACCCCA-3′
SEQ ID NO: 6-7, gRNA for mouse Il6 including 5′-AGGAACTTCATAGCGGTTTC-3′
(SEQ ID NO: 6) and 5′-ATGCTTAGGCATAACGCACT-3′ (SEQ ID NO: 7).
SEQ ID NO: 8-9, gRNA for mouse Tslp including 5′-CCACGTTCAGGCGACAGCAT-3′
(SEQ ID NO: 8) and 5′-TTATTCTGGAGATTGCATGA-3′ (SEQ ID NO: 9).
SEQ ID NO: 10-11, gRNA for mouse Ltbr including 5′-GCTCGGCTGACCAGACCGGG-3′
(SEQ ID NO: 10) and 5′-GAGCCACTGTTCTCACCTGG-3′ (SEQ ID NO: 11)
SEQ ID NO: 12-13, PCR primers for mouse Flt3 including
5′-GGTACCAGCAGAGTTGGATAGC-3′ (SEQ ID NO: 12) and
5′-ATCCCTTACACAGAAGCTGGAG-3′ (SEQ ID NO: 13)
SEQ ID NO: 14-17, PCR primers for human IL6 including
5′-CATCTCCTGTGGGACCATTCTTC-3′ (SEQ ID NO: 14),
5′-AGTGCAGGTTATCTCACTGTGG-3′ (SEQ ID NO: 15),
5′-TTGGAACTGAACCCAAGTGTGC-3′ (SEQ ID NO: 16), and
5′-GGCTGTCCTCAGACCCAATC-3′ (SEQ ID NO: 17).
SEQ ID NO: 18-19, PCR primers for human IL6 donor DNA backbone including
5′-GAAGTTTGTTGCTATGGAAGGGTC-3′ (SEQ ID NO: 18) and
5′-AGCGCAACGCAATTAATGTG-3′ (SEQ ID NO: 19)
SEQ ID NO: 20-23, PCR primers for human TSLP including
5′-CCTTCTCGTGTGAATAAGCTGC-3′ (SEQ ID NO: 20),
5′-CTCATCAGCATCTGCACACTTAG-3′ (SEQ ID NO: 21),
5′-CAGGGAGGTCTTGAAATCAGC-3′ (SEQ ID NO: 22), and
5′-CCAGGCTGTAGCATTTGGGTG-3′ (SEQ ID NO: 23).
SEQ ID NO: 24-27, PCR primers for human LTBR including
5′-GTGAAATGTATCTAGGGCCGCTC-3′ (SEQ ID NO: 24),
5′-TGCTCTGTCTCCGCTAGGTG-3′ (SEQ ID NO: 25),
5′-AGAGGTTCAGAGTTGTTCTCAGG-3′ (SEQ ID NO: 26), and
5′-ATGCGTCGGAGAACCAGACC-3′ (SEQ ID NO: 27).

### REFERENCES

Aspord, C., Pedroza-Gonzalez, A., Gallegos, M., Tindle, S., Burton, E. C., Su, D., . . . Palucka,
A. K. (2007). Breast cancer instructs dendritic cells to prime interleukin 13-secreting CD4+ T cells that facilitate tumor development. J Exp Med, 204(5), 1037-1047. doi:10.1084/jem.20061120
Audige, A., Rochat, M. A., Li, D., Ivic, S., Fahrny, A., Muller, C. K. S., . . . Speck, R. F. (2017). Long-term leukocyte reconstitution in NSG mice transplanted with human cord blood hematopoietic stem and progenitor cells. BMC Immunol, 18(1), 28. doi:10.1186/s12865-017-0209-9
Banchereau, J., & Steinman, R. M. (1998). Dendritic cells and the control of immunity. Nature, 392(6673), 245-252. doi:10.1038/32588
Billerbeck, E., Barry, W. T., Mu, K., Dorner, M., Rice, C. M., & Ploss, A. (2011). Development of human CD4+FoxP3+ regulatory T cells in human stem cell factor-, granulocyte-macrophage colony-stimulating factor-, and interleukin-3-expressing NOD-SCID IL2Rgamma(null) humanized mice. Blood, 117(11), 3076-3086. doi:10.1182/blood-2010-08-301507
Blunt, T., Finnie, N. J., Taccioli, G. E., Smith, G. C., Demengeot, J., Gottlieb, T. M., . . . Jackson, S. P. (1995). Defective DNA-dependent protein kinase activity is linked to V(D)J recombination and DNA repair defects associated with the murine scid mutation. Cell, 80(5), 813-823. doi:10.1016/0092-8674(95)90360-7
Cao, X., Shores, E. W., Hu-Li, J., Anver, M. R., Kelsall, B. L., Russell, S. M., . . . et al. (1995). Defective lymphoid development in mice lacking expression of the common cytokine receptor gamma chain. Immunity, 2(3), 223-238. doi:10.1016/1074-7613(95)90047-0
Carroll, D. (2011). Genome engineering with zinc-finger nucleases. Genetics, 188(4), 773-782. doi:10.1534/genetics.111.131433
Deltcheva, E., Chylinski, K., Sharma, C. M., Gonzales, K., Chao, Y., Pirzada, Z. A., . . . Charpentier, E. (2011). CRISPR RNA maturation by trans-encoded small RNA and host factor RNase III. Nature, 471(7340), 602-607. doi:10.1038/nature09886
Ding, Y., Wilkinson, A., Idris, A., Fancke, B., O'Keeffe, M., Khalil, D., . . . Radford, K. J. (2014). FLT3-ligand treatment of humanized mice results in the generation of large numbers of CD141+ and CD1c+ dendritic cells in vivo. J Immunol, 192(4), 1982-1989. doi:10.4049/jimmunol.1302391
Encabo, A., Mateu, E., Carbonell-Uberos, F., & Minana, M. D. (2003). IL-6 precludes the differentiation induced by IL-3 on expansion of CD34+ cells from cord blood. Haematologica, 88(4), 388-395.
Futterer, A., Mink, K., Luz, A., Kosco-Vilbois, M. H., & Pfeffer, K. (1998). The lymphotoxin beta receptor controls organogenesis and affinity maturation in peripheral lymphoid tissues. Immunity, 9(1), 59-70.
Gaj, T., Gersbach, C. A., & Barbas, C. F., 3rd. (2013). ZFN, TALEN, and CRISPR/Cas-based methods for genome engineering. Trends Biotechnol, 31(7), 397-405. doi:10.1016/j.tibtech.2013.04.004
Ginhoux, F., Liu, K., Helft, J., Bogunovic, M., Greter, M., Hashimoto, D., . . . Merad, M. (2009). The origin and development of nonlymphoid tissue CD103+ DCs. J Exp Med, 206(13), 3115-3130. doi:10.1084/jem.20091756
Gordon, J. W., & Ruddle, F. H. (1981). Integration and stable germ line transmission of genes injected into mouse pronuclei. Science, 214(4526), 1244-1246. doi:10.1126/science.6272397
Gossler, A., Doetschman, T., Korn, R., Serfling, E., & Kemler, R. (1986). Transgenesis by means of blastocyst-derived embryonic stem cell lines. Proc Natl Acad Sci USA, 83(23), 9065-9069. doi:10.1073/pnas.83.23.9065
Greiner, D. L., Hesselton, R. A., & Shultz, L. D. (1998). SCID mouse models of human stem cell engraftment. Stem Cells, 16(3), 166-177. doi:10.1002/stem.160166
Hagai, T., Chen, X., Miragaia, R. J., Rostom, R., Gomes, T., Kunowska, N., . . . Teichmann, S. A. (2018). Gene expression variability across cells and species shapes innate immunity. Nature, 563(7730), 197-202. doi:10.1038/s41586-018-0657-2
Hanabuchi, S., Ito, T., Park, W. R., Watanabe, N., Shaw, J. L., Roman, E., . . . Liu, Y. J. (2010). Thymic stromal lymphopoietin-activated plasmacytoid dendritic cells induce the generation of FOXP3+ regulatory T cells in human thymus. J Immunol, 184(6), 2999-3007. doi:10.4049/jimmunol.0804106
Hanabuchi, S., Watanabe, N., & Liu, Y. J. (2012). TSLP and immune homeostasis. Allergol Int, 61(1), 19-25. doi:10.2332/allergolint.11-RAI-0394
Harms, D. W., Quadros, R. M., Seruggia, D., Ohtsuka, M., Takahashi, G., Montoliu, L., & Gurumurthy, C. B. (2014). Mouse Genome Editing Using the CRISPR/Cas System. Curr Protoc Hum Genet, 83, 15 17 11-27. doi:10.1002/0471142905.hg1507s83
He, R., & Geha, R. S. (2010). Thymic stromal lymphopoietin. Ann NY Acad Sci, 1183, 13-24. doi:10.1111/j.1749-6632.2009.05128.x
Herndler-Brandstetter, D., Shan, L., Yao, Y., Stecher, C., Plajer, V., Lietzenmayer, M., . . . Flavell, R. A. (2017). Humanized mouse model supports development, function, and tissue residency of human natural killer cells. Proc Natl Acad Sci USA, 114(45), E9626-E9634. doi:10.1073/pnas.1705301114
Inui, M., Miyado, M., Igarashi, M., Tamano, M., Kubo, A., Yamashita, S., . . . Takada, S. (2014). Rapid generation of mouse models with defined point mutations by the CRISPR/Cas9 system. Sci Rep, 4, 5396. doi:10.1038/srep05396
Ito, T., Wang, Y. H., Duramad, O., Hori, T., Delespesse, G. J., Watanabe, N., . . . Liu, Y. J. (2005). TSLP-activated dendritic cells induce an inflammatory T helper type 2 cell response through OX40 ligand. J Exp Med, 202(9), 1213-1223. doi:10.1084/jem.20051135
Jaenisch, R. (1976). Germ line integration and Mendelian transmission of the exogenous Moloney leukemia virus. Proc Natl Acad Sci USA, 73(4), 1260-1264. doi:10.1073/pnas.73.4.1260
Jego, G., Palucka, A. K., Blanck, J. P., Chalouni, C., Pascual, V., & Banchereau, J. (2003). Plasmacytoid dendritic cells induce plasma cell differentiation through type I interferon and interleukin 6. Immunity, 19(2), 225-234.
Jinek, M., Chylinski, K., Fonfara, I., Hauer, M., Doudna, J. A., & Charpentier, E. (2012). A programmable dual-RNA-guided DNA endonuclease in adaptive bacterial immunity. Science, 337(6096), 816-821. doi:10.1126/science.1225829
Joung, J. K., & Sander, J. D. (2013). TALENs: a widely applicable technology for targeted genome editing. Nat Rev Mol Cell Biol, 14(1), 49-55. doi:10.1038/nrm3486
Kanazawa, N. (2007). Dendritic cell immunoreceptors: C-type lectin receptors for pattern-recognition and signaling on antigen-presenting cells. J Dermatol Sci, 45(2), 77-86.
Karsunky, H., Merad, M., Cozzio, A., Weissman, I. L., & Manz, M. G. (2003). Flt3 ligand regulates dendritic cell development from Flt3+ lymphoid and myeloid-committed progenitors to Flt3+ dendritic cells in vivo. J Exp Med, 198(2), 305-313.
Krautler, N. J., Kana, V., Kranich, J., Tian, Y., Perera, D., Lemm, D., . . . Aguzzi, A. (2012). Follicular dendritic cells emerge from ubiquitous perivascular precursors. Cell, 150(1), 194-206. doi:10.1016/j.cell.2012.05.032
Lu, N., Wang, Y. H., Wang, Y. H., Arima, K., Hanabuchi, S., & Liu, Y. J. (2009). TSLP and IL-7 use two different mechanisms to regulate human CD4⁺ T cell homeostasis. J Exp Med, 206(10), 2111-2119. doi:10.1084/jem.20090153
Makino, S., Kunimoto, K., Muraoka, Y., Mizushima, Y., Katagiri, K., & Tochino, Y. (1980). Breeding of a non-obese, diabetic strain of mice. Jikken Dobutsu, 29(1), 1-13. 50 doi:10.1538/expaniml978.29.1_1
Maraskovsky, E., Brasel, K., Teepe, M., Roux, E. R., Lyman, S. D., Shortman, K., & McKenna, H. J. (1996). Dramatic increase in the numbers of functionally mature dendritic cells in Flt3 ligand-treated mice: multiple dendritic cell subpopulations identified. J Exp Med, 184(5), 1953-1962.
Matsumura, T., Kametani, Y., Ando, K., Hirano, Y., Katano, I., Ito, R., . . . Habu, S. (2003). Functional CD5⁺ B cells develop predominantly in the spleen of NOD/SCID/gammac(null) (NOG) mice transplanted either with human umbilical cord blood, bone marrow, or mobilized peripheral blood CD34⁺ cells. Exp Hematol, 31(9), 789-797.
McKenna, H. J., Stocking, K. L., Miller, R. E., Brasel, K., De Smedt, T., Maraskovsky, E., . . . Peschon, J. J. (2000). Mice lacking flt3 ligand have deficient hematopoiesis affecting hematopoietic progenitor cells, dendritic cells, and natural killer cells. Blood, 95(11), 3489-3497.
Mestas, J., & Hughes, C. C. (2004). Of mice and not men: differences between mouse and human immunology. J Immunol, 172(5), 2731-2738.
Nicolini, F. E., Cashman, J. D., Hogge, D. E., Humphries, R. K., & Eaves, C. J. (2004). NOD/SCID mice engineered to express human IL-3, GM-CSF and Steel factor constitutively mobilize engrafted human progenitors and compromise human stem cell regeneration. Leukemia, 18(2), 341-347. doi:10.1038/sj.leu.2403222
Nurieva, R. I., Chung, Y., Martinez, G. J., Yang, X. O., Tanaka, S., Matskevitch, T. D., . . . Dong, C. (2009). Bcl6 mediates the development of T follicular helper cells. Science, 325(5943), 1001-1005. doi:10.1 126/science.1176676
Pedroza-Gonzalez, A., Xu, K., Wu, T. C., Aspord, C., Tindle, S., Marches, F., . . . Palucka, A. K. (2011). Thymic stromal lymphopoietin fosters human breast tumor growth by promoting type 2 inflammation. J Exp Med, 208(3), 479-490. doi:10.1084/jem.20102131
Pulendran, B., Banchereau, J., Burkeholder, S., Kraus, E., Guinet, E., Chalouni, C., . . . Palucka, K. (2000). Flt3-ligand and granulocyte colony-stimulating factor mobilize distinct human dendritic cell subsets in vivo. J Immunol, 165(1), 566-572.
Rathinam, C., Poueymirou, W. T., Rojas, J., Murphy, A. J., Valenzuela, D. M., Yancopoulos, G. D., . . . Flavell, R. A. (2011). Efficient differentiation and function of human macrophages in humanized CSF-1 mice. Blood, 118(11), 3119-3128. doi:10.1182/blood-2010-12-326926
Rongvaux, A., Willinger, T., Martinek, J., Strowig, T., Gearty, S. V., Teichmann, L. L., . . . Flavell, R. A. (2014). Development and function of human innate immune cells in a humanized mouse model. Nat Biotechnol, 32(4), 364-372. doi:10.1038/nbt.2858
Saito, Y., Ellegast, J. M., Rafiei, A., Song, Y., Kull, D., Heikenwalder, M., . . . Manz, M. G. (2016). Peripheral blood CD34(+) cells efficiently engraft human cytokine knock-in mice. Blood, 128(14), 1829-1833. doi:10.1182/blood-2015-10-676452
Shultz, L. D., Brehm, M. A., Garcia-Martinez, J. V., & Greiner, D. L. (2012). Humanized mice for immune system investigation: progress, promise and challenges. Nat Rev Immunol, 12(11), 786-798. doi:10.1038/nri3311
Shultz, L. D., Ishikawa, F., & Greiner, D. L. (2007). Humanized mice in translational biomedical research. Nat Rev Immunol, 7(2), 118-130. doi:10.1038/nri2017
Shultz, L. D., Lyons, B. L., Burzenski, L. M., Gott, B., Chen, X., Chaleff, S., . . . Handgretinger, R. (2005). Human lymphoid and myeloid cell development in NOD/LtSz-scid IL2R gamma null mice engrafted with mobilized human hemopoietic stem cells. J Immunol, 174(10), 6477-6489. doi: 10.4049/jimmunol.174.10.6477
Shultz, L. D., Schweitzer, P. A., Christianson, S. W., Gott, B., Schweitzer, I. B., Tennent, B., . . . et al. (1995). Multiple defects in innate and adaptive immunologic function in NOD/LtSz-scid mice. J Immunol, 154(1), 180-191.
Theocharides, A. P., Rongvaux, A., Fritsch, K., Flavell, R. A., & Manz, M. G. (2016). 50 Humanized hemato-lymphoid system mice. Haematologica, 101(1), 5-19. doi:10.3324/haematol.2014.115212
Traggiai, E., Chicha, L., Mazzucchelli, L., Bronz, L., Piffaretti, J. C., Lanzavecchia, A., & Manz, M. G. (2004). Development of a human adaptive immune system in cord blood cell-transplanted mice. Science, 304(5667), 104-107. doi:10.1126/science.1093933
Victor Garcia, J. (2016). Humanized mice for HIV and AIDS research. Curr Opin Virol, 19, 56-64. doi:10.1016/j.coviro.2016.06.010
Wang, H., Yang, H., Shivalila, C. S., Dawlaty, M. M., Cheng, A. W., Zhang, F., & Jaenisch, R. (2013). One-step generation of mice carrying mutations in multiple genes by CRISPR/Cas-mediated genome engineering. Cell, 153(4), 910-918. doi:10.1016/j.cell.2013.04.025
Waskow, C., Liu, K., Darrasse-Jeze, G., Guermonprez, P., Ginhoux, F., Merad, M., . . . Nussenzweig, M. (2008). The receptor tyrosine kinase Flt3 is required for dendritic cell development in peripheral lymphoid tissues. Nat Immunol, 9(6), 676-683. doi:10.1038/ni.1615
Williams, A., Flavell, R. A., & Eisenbarth, S. C. (2010). The role of NOD-like Receptors in shaping adaptive immunity. Curr Opin Immunol, 22(1), 34-40. doi:10.1016/j.coi.2010.01.004
Willinger, T., Rongvaux, A., Takizawa, H., Yancopoulos, G. D., Valenzuela, D. M., Murphy, A. J., . . . Flavell, R. A. (2011). Human IL-3/GM-CSF knock-in mice support human alveolar macrophage development and human immune responses in the lung. Proc Natl Acad Sci USA, 108(6), 2390-2395. doi:10.1073/pnas.1019682108
Wu, T. C., Xu, K., Banchereau, R., Marches, F., Yu, C. I., Martinek, J., . . . Palucka, K. (2014). Reprogramming tumor-infiltrating dendritic cells for CD103+CD8+ mucosal T-cell differentiation and breast cancer rejection. Cancer Immunol Res, 2(5), 487-500. doi:10.1158/2326-6066.CIR-13-0217
Wu, T. C., Xu, K., Martinek, J., Young, R. R., Banchereau, R., George, J., . . . Palucka, A. K. (2018). IL1 Receptor Antagonist Controls Transcriptional Signature of Inflammation in Patients with Metastatic Breast Cancer. Cancer Res, 78(18), 5243-5258. doi:10.1158/0008-5472.CAN-18-0413
Wunderlich, M., Chou, F. S., Link, K. A., Mizukawa, B., Perry, R. L., Carroll, M., & Mulloy, J. C. (2010). AML xenograft efficiency is significantly improved in NOD/SCID-IL2RG mice constitutively expressing human SCF, GM-CSF and IL-3. Leukemia, 24(10), 1785-1788. doi:10.1038/leu.2010.158
Yang, H., Wang, H., Shivalila, C. S., Cheng, A. W., Shi, L., & Jaenisch, R. (2013). One-step generation of mice carrying reporter and conditional alleles by CRISPR/Cas-mediated genome engineering. Cell, 154(6), 1370-1379. doi:10.1016/j.cell.2013.08.022
Yu, C. I., Gallegos, M., Marches, F., Zurawski, G., Ramilo, O., Garcia-Sastre, A., . . . Palucka, A. K. (2008). Broad influenza-specific CD8⁺ T-cell responses in humanized mice vaccinated with influenza virus vaccines. Blood, 112(9), 3671-3678. doi:10.1182/blood-2008-05-157016
Zhang, L., & Su, L. (2012). HIV-1 immunopathogenesis in humanized mouse models. Cell Mol Immunol, 9(3), 237-244. doi:10.1038/cmi.2012.7

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A non-obese diabetic (NOD) mouse comprising an inactivated mouse Prkdc allele, an inactivated mouse IL2rg allele, and an inactivated mouse Flt3 allele.

2. The mouse of claim 1, wherein the mouse is a NOD.Cg-Prkdc^{scid} Il2rg^{tm1Wjl}/SzJ (NOD scid gamma) mouse comprising an inactivated mouse Flt3 allele.

3. A method of producing the mouse of claim 2 comprising inactivating a mouse Flt3 allele in a NOD scid gamma mouse.

4. A method of producing the mouse of claim 1 or 2 comprising
(a) developing founder mice that have a NOD scid gamma genetic background and an inactivated mouse Flt3 allele; and
(b) interbreeding the founder mice to produce progeny mice homozygous for the inactivated mouse Flt3 allele.

5. A method of producing the mouse of claim 1 or 2, comprising
(a) coinjecting Cas9 mRNA or Cas9 protein and a gRNA targeting mouse Flt3 into fertilized NOD scid gamma oocytes, wherein a mouse Flt3 allele is inactivated; and
(b) breeding the founder mice to NOD scid gamma mice to produce F1 progeny mice; and
(c) interbreeding the F1 progeny mice to produce F2 progeny mice homozygous for the inactivated mouse Flt3 allele.

6. A method comprising breeding female mice homozygous for Prkdc^{scid}, homozygous for Il2rg^{tm1Wjl} and homozygous for Flt3^{em1Akp} with male mice homozygous for Prkdc^{scid}, hemizygous for the X-linked Il2rg^{tm1Wjl} and homozygous for Flt3^{em1Akp} to produce progeny mice.

7. A gRNA targeting mouse Flt3, optionally wherein the gRNA comprises the sequence of SEQ ID NO: 5.

8. A mouse oocyte comprising the gRNA of claim 7, optionally wherein the mouse oocyte is fertilized.

9. The mouse oocyte of claim 8 further comprising Cas9 mRNA and/or Cas9 protein.

10. The mouse of claim 1 or 2 further comprising a nucleic acid encoding human thymic stromal lymphopoietin (TSLP).

11. The mouse of claim 10, wherein the nucleic acid encoding human TSLP comprises a human TSLP transgene.

12. The mouse of claim 11, wherein the human TSLP transgene comprises a nucleic acid sequence of SEQ ID NO: 3.

13. The mouse of any one of claims 10-12, wherein the mouse expresses human TSLP.

14. The mouse of any one of claims 10-13, wherein the mouse comprises an inactivated mouse Tslp allele and/or does not express mouse Tslp.

15. A method of producing the mouse of any one of claims 10-14 comprising inactivating a mouse Flt3 allele in a NOD scid gamma mouse and introducing the nucleic acid encoding human TSLP.

16. A method of producing the mouse of any one of claims 10-14, comprising
(a) developing founder mice that have a NOD scid gamma genetic background, an inactivated mouse Tslp, and a nucleic acid encoding human TSLP; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for a nucleic acid encoding human TSLP.

17. A method of producing the mouse of any one of claim 2024, comprising
(a) coinjecting Cas9 mRNA or Cas9 protein, a gRNA targeting mouse Tslp, and a nucleic acid encoding human TSLP into fertilized NOD scid gamma oocytes that comprise an inactivated mouse Flt3 allele, wherein the nucleic acid encoding human TSLP is genomically inserted via homologous recombination to produce founder mice; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for the nucleic acid encoding human TSLP.

18. A method comprising breeding female mice homozygous for Prkdc^{scid}, homozygous for Il2rg^{tm1Wjl}, homozygous for Flt3^{em1Akp}, and homozygous for Tslp^{em3(TSLP)Akp} with male mice homozygous for Prkdc^{scid}, hemizygous for the X-linked Il2rg^{tm1Wjl} homozygous for Flt3^{em1Akp}, homozygous for Tslp^{em3(TSLP)Akp} and to produce progeny mice.

19. A gRNA targeting mouse Tslp, optionally wherein the gRNA comprises the sequence of SEQ ID NO: 8 or SEQ ID NO: 9.

20. A mouse oocyte comprising the gRNA of claim 19, optionally wherein the mouse oocyte is fertilized.

21. The mouse oocyte of claim 20 further comprising Cas9 mRNA and/or Cas9 protein.

22. The mouse oocyte of claim 21 further comprising a nucleic acid encoding human TSLP.

23. The mouse of claim 1 or 2 further comprising a nucleic acid encoding human interleukin 6 (IL6).

24. The mouse of claim 23, wherein the nucleic acid encoding human IL6 comprises a human IL6 transgene.

25. The mouse of claim 24, wherein the human IL6 transgene comprises a nucleic acid sequence of SEQ ID NO: 2.

26. The mouse of any one of claims 23-25, wherein the mouse expresses human IL6.

27. The mouse of any one of claims 23-26, wherein the mouse comprises an inactivated mouse IL6 allele and/or does not express mouse IL6.

28. A method of producing the mouse of any one of claims 23-27 comprising inactivating a mouse Flt3 allele in a NOD scid gamma mouse and introducing the nucleic acid encoding human IL6.

29. A method of producing the mouse of any one of claims 23-27, comprising
(a) developing founder mice that have a NOD scid gamma genetic background, an inactivated mouse IL6, and a nucleic acid encoding human IL6; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for a nucleic acid encoding human IL6.

30. A method of producing the mouse of any one of claims 23-27, comprising
(a) coinjecting Cas9 mRNA or Cas9 protein, a gRNA targeting mouse Il6, and a nucleic acid encoding human IL6 into fertilized NOD scid gamma oocytes that comprise an inactivated mouse Flt3 allele, wherein the nucleic acid encoding human IL6 is genomically inserted via homologous recombination to produce founder mice; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for the nucleic acid encoding human IL6.

31. A method comprising breeding female mice homozygous for Prkdc^{scid}, homozygous for Il2rg^{tm1Wjl}, homozygous for Flt3^{em1Akp}, and homozygous for Il6^{em3(IL6)Akp} with male mice homozygous for Prkdc^{scid}, hemizygous for the X-linked Il2rg^{tm1Wjl}, homozygous for Flt3^{em1Akp}homozygous for Il6^{em3(IL6)Akp} and to produce progeny mice.

32. A gRNA targeting mouse Il6, optionally wherein the gRNA comprises the sequence of SEQ ID NO: 6 or SEQ ID NO: 7.

33. A mouse oocyte comprising the gRNA of claim 32, optionally wherein the mouse oocyte is fertilized.

34. The mouse oocyte of claim 33 further comprising Cas9 mRNA and/or Cas9 protein.

35. The mouse oocyte of claim 34 further comprising a nucleic acid encoding human IL6.

36. The mouse of claim 1 or 2 further comprising a nucleic acid encoding human lymphotoxin beta receptor (LTBR).

37. The mouse of claim 36, wherein the nucleic acid encoding human LTBR comprises a human LTBR transgene.

38. The mouse of claim 37, wherein the human LTBR transgene comprises a nucleic acid sequence of SEQ ID NO: 4

39. The mouse of any one of claims 36-38, wherein the mouse expresses human LTBR.

40. The mouse of any one of claims 36-39, wherein the mouse comprises an inactivated mouse Ltbr allele and/or does not express mouse Ltbr.

41. A method of producing the mouse of any one of claims 36-40 comprising inactivating a mouse Flt3 allele in a NOD scid gamma mouse and introducing the nucleic acid encoding human LTBR.

42. A method of producing the mouse of any one of claims 36-40, comprising
(a) developing founder mice that have a NOD scid gamma genetic background, an inactivated mouse Ltbr, and a nucleic acid encoding human LTBR; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for a nucleic acid encoding human LTBR.

43. A method of producing the mouse of any one of claims 36-40, comprising
(a) coinjecting Cas9 mRNA or Cas9 protein, a gRNA targeting mouse Ltbr, and a nucleic acid encoding human LTBR into fertilized NOD scid gamma oocytes that comprise an inactivated mouse Flt3 allele, wherein the nucleic acid encoding human LTBR is genomically inserted via homologous recombination to produce founder mice; and
(b) breeding the founder mice to NOD scid gamma mice that comprise an inactivated mouse Flt3 allele to produce F1 progeny mice; and
(c) interbreeding the F1 progeny mice to produce F2 progeny mice homozygous for the nucleic acid encoding human LTBR.

44. A method comprising breeding female mice homozygous for Prkdc^{scid}, homozygous for Il2rg^{tm1Wjl}, homozygous for Flt3^{em1Akp}, and homozygous for Ltbr^{em1(LTBR)Akp} with male mice homozygous for Prkdc^{scid}, hemizygous for the X-linked Il2rg^{tm1Wjl} homozygous for Flt3^{em1Akp}, homozygous for Ltbr^{em1(LTBR)Akp} and to produce progeny mice.

45. A gRNA targeting mouse Ltbr, optionally wherein the gRNA comprises the sequence of SEQ ID NO: 10 or SEQ ID NO: 11.

46. A mouse oocyte comprising the gRNA of claim 45, optionally wherein the mouse oocyte is fertilized.

47. The mouse oocyte of claim 46 further comprising Cas9 mRNA and/or Cas9 protein.

48. The mouse oocyte of claim 47 further comprising a nucleic acid encoding human LTBR.

49. The mouse of claim 1 or 2 further comprising: a nucleic acid encoding human interleukin 3 (IL3); a nucleic acid encoding human granulocyte/macrophage-stimulating factor (GM-CSF); and a nucleic acid encoding human Stem cell factor (SCF).

50. The mouse of claim 49, wherein (a) the nucleic acid encoding human IL3 comprises a human IL3 transgene; (b) the nucleic acid encoding human GM-CSF comprises a human GM-CSF transgene; and (c) the nucleic acid encoding human SF comprises a human SF transgene.

51. The mouse of claim 49 or 50 wherein the mouse expresses human IL3, human GM-CSF, and human SF.

52. A method of producing the mouse of any one of claims 49-51 comprising introducing a nucleic acid encoding human interleukin 3 (IL3), a nucleic acid encoding human granulocyte/macrophage-stimulating factor (GM-CSF), and a nucleic acid encoding human Stem cell factor (SF) into a NOD scid gamma mouse that comprises an inactivated mouse Flt3 allele.

53. A method of producing the mouse of any one of claims 49-51 comprising crossing a NOD scid gamma mouse that comprises an inactivated mouse Flt3 allele with a NOD scid gamma mouse that comprises a nucleic acid encoding human interleukin 3 (IL3), a nucleic acid encoding human granulocyte/macrophage-stimulating factor (GM-CSF), and a nucleic acid encoding human Stem cell factor (SF).

54. A method comprising breeding female mice homozygous for Prkdc^{scid} homozygous for Il2rg^{tm1Wjl}, homozygous for Flt3^{em1Akp}, homozygous for IL3, homozygous for GM-CSF, and homozygous for SF with male mice homozygous for Prkdc^{scid}, hemizygous for the X-linked Il2rg^{tm1Wjl}, homozygous for Flt3^{em1Akp}, homozygous for IL3, homozygous for GM-CSF, and homozygous for SF and to produce progeny mice.

55. A cell obtained from the mouse of any one of the preceding claims.

56. A mouse comprising a cell having the same genotype of a cell obtained from the mouse of any one of the preceding claims.

57. A progeny mouse of the mouse of any one of the preceding claims.

58. A method of producing the mouse of any one of the preceding claims.

59. A method of propagating the mouse of any one of the preceding claims.

60. The method of claim 59 comprising breeding the mouse of any one of the preceding claims with a second mouse to produce a progeny mouse.

61. The method of claim 60, wherein the second mouse is mouse of any one of the preceding claims.

62. A method of using the mouse of any one of the preceding claims

63. The method of claim 62 comprising: sublethally irradiating the mouse; and injecting the mouse with human CD34+ hematopoietic stem cells.

64. The method of claim 63 further comprising administering to the mouse an agent of interest.

65. The method of claim 64 further comprising assessing an effect of the agent on human immune cells in the mouse.

66. The method of claim 65, wherein the human immune cells are selected from T cells, dendritic cells, natural killer cells, and macrophages.

67. A NOD.Cg-Prkdc^{scid} Il2rg^{tm1Wjl}/SzJ mouse comprising
an inactivated mouse Flt3 allele.

68. A NOD.Cg-Prkdc^{scid} Il2rg^{tm1Wj1}/SzJ mouse comprising
an inactivated mouse Flt3 allele and
a nucleic acid encoding human thymic stromal lymphopoietin (TSLP).

69. A NOD.Cg-Prkdc^{scid} Il2rg^{tm1Wjl}/SzJ mouse comprising
an inactivated mouse Flt3 allele and
a nucleic acid encoding human interleukin 6 (IL6).

70. A NOD.Cg-Prkdc^{scid} Il2rg^{tm1Wjl}/SzJ mouse comprising
an inactivated mouse Flt3 allele and
a nucleic acid encoding human lymphotoxin beta receptor (LTBR).

71. A NOD.Cg-Prkdc^{scid} Il2rg^{tm1Wjl}/SzJ mouse comprising
an inactivated mouse Flt3 allele,
a nucleic acid encoding human interleukin 3 (IL3),
a nucleic acid encoding human granulocyte/macrophage-stimulating factor (GM-CSF),
and
a nucleic acid encoding human Stem cell factor (SF).
